(19) United States
(12) Patent Application Publication
(10) Pub. No.: US 2019/0255080 A1
(43) Pub. Date: Aug. 22, 2019

(54) METHOD OF REDUCING THYROID-ASSOCIATED SIDE EFFECTS

(71) Applicants: Viking Therapeutics, Inc., San Diego, CA (US); Metabasis Therapeutics, Inc., San Diego, CA (US)

(72) Inventors: Brian Lian, Rancho Santa Fe, CA (US); Hiroko Masamune, San Diego, CA (US); Mark Erion, Lansdale, PA (US); Bruce Ito, San Diego, CA (US)

(21) Appl. No.: 16/333,513

(22) PCT Filed: Sep. 13, 2017
(86) PCT No.: PCT/US2017/051410

§ 371 Date: Mar. 14, 2019

Related U.S. Application Data
(60) Provisional application No. 62/396,015, filed on Sep. 16, 2016.
(60) Provisional application No. 62/396,025, filed on Sep. 16, 2016.

(51) Int. Cl. A61K 31/662; A61K 31/665; A61P 3/06; A61P 3/04; A61P 3/10; A61P 1/16; A61P 9/10; A61P 5/14

(52) U.S. Cl. CPC A61K 31/662; A61K 31/665; A61P 3/06; A61P 5/14; A61P 3/10; A61P 1/16; A61P 9/10; A61P 3/04

(57) ABSTRACT

The present disclosure is directed to methods of administration of thyroid hormone receptor agonists. The disclosure provides methods wherein the activity of the given thyroid receptor agonists in ameliorating or curing obesity, hyperlipidemia, hypercholesterolemia, diabetes, non-alcoholic fatty liver disease, non-alcoholic steatohepatitis, atherosclerosis, cardiovascular disease, hypothyroidism, and related disorders is maintained, while thyroid-related and thyroid axis-related side effects are reduced or eliminated.

### BACKGROUND

### Field of the Invention

The compounds and methods described herein relate generally to the field of treatment of thyroid-mediated disorders, and specifically to mechanisms of reducing side effects from the administration of thyroid hormone receptor agonists.

### Description of the State of the Art

The thyroid hormones (THs) play a critical role in growth, development, metabolism, and homeostasis. They are produced by the thyroid gland as thyroxine (T4) and 3,5,3′-triiodo-L-thyronine (T3). T4 is the major secreted form in humans and is enzymatically deiodinated by deiodinases to the more active form, T3, in peripheral tissues. THs exert their action by interacting with thyroid hormone receptors (TRs), which belong to the nuclear hormone receptor superfamily, and regulate the transcription of target genes. TH's form part of the thyroid axis, also known as the Hypothalmic-Pituitary-Thyroid, or HPT axis, which comprises a complex endocrine and paracrine feedback loop linking tissues of the brain and endocrine system in order to assert global control over issues such as overall metabolic rate, lipid secretion, cardiac function, muscle and bone growth, among many others (See e.g., Robins and Cotran: Pathologic Basis of Disease, Kumar, V. et al., eds. (2005), p. 1165, which is incorporated herein by reference in its entirety).

TRs are expressed in most tissues and exist as two isoforms (TRα and TRPβ). Tissue distribution studies, mouse knockout studies, and evaluation of patients with resistance to thyroid hormone (RTH) syndrome have established that TRα is the predominant isoform in the heart and regulates most cardiac functions, while the TRPβ isoform predominates in the liver and the pituitary and regulates cholesterol metabolism and thyroid stimulating hormone (TSH) production, respectively. In recognition of the potential benefits associated with modulation of TRs, numerous approaches have been pursued to identify a suitable TR agonist to lower plasma cholesterol levels. However, these benefits were offset by deleterious cardiovascular side effects, such as tachycardia, arrhythmia, elevated blood pressure, and heart failure as well as effects on the thyroid hormone axis, muscle metabolism and bone loss.

TR-mediated pathways are implicated in modulating serum lipid levels, including cholesterol, triglycerides, and associated lipoproteins. See Pearce, E. N., Curr. Cardiol. Rep. 6:451-6 (2004) and Duntas, L. H., Thyroid 12:287-93 (2002) both of which are incorporated herein by reference in their entireties. Elevated levels of serum lipids are implicated in the development of atherosclerosis and in the exacerbation of coronary artery disease. See Robins and Cotran: Pathologic Basis of Disease, Kumar, V. et al., eds. (2005), p. 523, 572-77, which is incorporated herein by reference in its entirety. Clinical trials have demonstrated that reducing low density lipoprotein/serum cholesterol levels reduces morbidity and mortality associated with cardiovascular disease. See Grundy, S. M., et al., Circulation 110:227-39 (2004), which is incorporated herein by reference in its entirety. While drugs such as statins and PCSK-9 inhibitors, along with dietary and lifestyle interventions, may help to treat hyperlipidemia in some patients, many patients fail to significantly reduce their serum cholesterol levels and many do not tolerate high doses of statins. See Pearson, T. et al., Arch Intern Med. 160:459-467 (2000), which is incorporated herein by reference in its entirety. Thus, there is an unmet medical need for additional orally administered lipid-modulating therapies.

Similarly, nonalcoholic fatty liver disease (NAFLD), a condition linked to the group of metabolic irregularities known as metabolic syndrome, is defined by excessive fat accumulation in the form of triglycerides (steatosis) in the liver. This condition can further include liver cell injury and inflammation, leading to non-alcoholic steatohepatitis (NASH). NASH generally coincides in patients with type 2 diabetes, hypercholesterolemia, hypertriglyceridemia, and obesity. Patients with NASH risk developing cirrhosis, liver failure, and hepatocellular carcinoma. Treatments for NASH are currently limited to lifestyle interventions. However, the role of thyroid hormone in regulating LDL-C and triglyceride levels makes TR-mediated pathways promising targets for treatments for NASH and NAFLD. For example, in animals, thyroid hormone mimetics have been shown to dramatically reduce liver fat content.

Selective TRPβ agonists were developed as a means of suppressing the cardiac side effects of nonspecific TR agonists while retaining the potential beneficial effects of TRPβ activation, such as reduction in cholesterol and serum lipid levels, and reduction in obesity due to increased cellular metabolism. See Fujitaki, J. M., et al., Drug Metab. Disp. 36(11) 2393-403 (2008)), which is hereby incorporated by reference in its entirety. However, it has been shown that even targeted TRPβ agonists can lead to suppression of the thyroid hormone axis (see Erion, M. D., PNAS USA 104(39):15490-5 (2007), which is incorporated herein by reference in its entirety), which may lead to side effects ranging from depression and fatigue to muscle wasting and bone loss. Accordingly, there is a need for compositions and methods to effect TRPβ activation while reducing HPT axis suppression and its associated side effects.

### SUMMARY OF THE DISCLOSURE

The present disclosure provides a method of treating a condition such as obesity, hyperlipidemia, hypercholesterolemia, diabetes, non-alcoholic fatty liver disease, non-alcoholic steatohepatitis, atherosclerosis, cardiovascular disease, hypothyroidism, and thyroid cancer; in a subject in need thereof, by administering a therapeutically effective amount of one or more compounds such as the following:

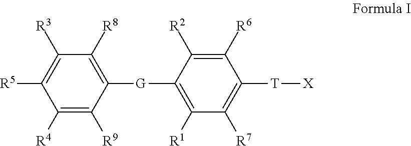

wherein:

G is selected from the group consisting of —O—, —S—, —S(═O)—, —S(═O)₂—, —Se—, —CH₂—, —CF₂—, —CHF—, —C(O)—, —CH(OH)—, —CH(C₁-C₄ alkyl)-, —CH(C₁-C₄ alkoxy)-, —C(═CH₂)—, —NH—, and —N(C₁-C₄ alkyl)-;

T is selected from the group consisting of —(CR^{a}₂)ₖ—, —CR^{b}═CR^{b}—(CR^{a}₂)ₙ—, (CR^{a}₂)ₙ—CR^{b}═CR^{b}, (CR^{a}₂)—CR^{b}═CR^{b}—(CR^{a}₂), —O(CR^{b}₂)(CR^{a}₂)ₙ—, —S(CR^{b}₂)(CR^{a}₂)ₙ—, N(R^{c})(CR^{b}₂)(CR^{a}₂)ₙ—, N(R^{b})C(O)(CR^{a}₂)ₙ, —C(O)(CR^{a}₂)^{m}, —(CR^{a}₂)ₘC(O)—, —(CR^{a}₂)C(O)(CR^{a}₂)ₙ, —(CR^{a}₂)ₙC(O)(CR^{a}₂)—, and —C(O)NH(CR^{b}₂)(CR^{a}₂)ₚ—;

k is an integer from 1-4;

m is an integer from 0-3;

n is an integer from 0-2;

p is an integer from 0-1;

each R^{a} is independently selected from the group consisting of hydrogen, optionally substituted —C₁-C₄ alkyl, halogen, —OH, optionally substituted —O—C₁-C₄ alkyl, —OCF₃, optionally substituted —S—C₁-C₄ alkyl, —NR^{b}R^{c}, optionally substituted —C₂-C₄ alkenyl, and optionally substituted —C₂-C₄ alkynyl; with the proviso that when one R^{a} is attached to C through an O, S, or N atom, then the other R^{a} attached to the same C is a hydrogen, or attached via a carbon atom;

each R^{b} is independently selected from the group consisting of hydrogen and optionally substituted —C₁-C₄ alkyl;

each R^{c} is independently selected from the group consisting of hydrogen and optionally substituted —C₁-C₄ alkyl, optionally substituted —C(O)—C₁-C₄ alkyl, and —C(O)H;

R¹, and R² are each independently selected from the group consisting of halogen, optionally substituted —C₁-C₄ alkyl, optionally substituted —S—C₁-C₃ alkyl, optionally substituted —C₂-C₄ alkenyl, optionally substituted —C₂-C₄ alkynyl, —CF₃, —OCF₃, optionally substituted-O—C₁-C₃ alkyl, and cyano;

R⁶, R⁷, R⁸, and R⁹ are each independently selected from the group consisting of hydrogen, halogen, optionally substituted —C C₁-C₄ alkyl, optionally substituted —S—C₁-C₃ alkyl, optionally substituted —C₂-C₄ alkenyl, optionally substituted —C₂-C₄ alkynyl, —CF₃, —OCF₃, optionally substituted-O—C₁-C₃ alkyl, and cyano; or R⁶ and T are taken together along with the carbons they are attached to form a ring of 5 to 6 atoms including 0 to 2 heteroatoms independently selected from —NRⁱ—, —O—, and —S—, with the proviso that when there are 2 heteroatoms in the ring and both heteroatoms are different than nitrogen then both heteroatoms have to be separated by at least one carbon atom; and X is attached to this ring by a direct bond to a ring carbon, or by —(CR^{a}₂)— or —C(O)— bonded to a ring carbon or a ring nitrogen;

Rⁱ is selected from the group consisting of hydrogen, —C(O)C₁-C₄ alkyl, —C₁-C₄ alkyl, and —C₁-C₄-aryl;

R³ and R⁴ are independently selected from the group consisting of hydrogen, halogen, —CF₃, —OCF₃, cyano, optionally substituted —C₁-C₁₂ alkyl, optionally substituted —C₂-C₁₂ alkenyl, optionally substituted —C₂-C₁₂ alkynyl, —SR^{d}, —S(═O)R^{e}, —S(═O)₂R^{e}, —S(═O)₂NR^{f}R^{g}, —C(O)OR^{h}, —C(O)R^{e}, —N(R^{b})C(O)NR^{f}R^{g}, —N(R^{b})S(═O)₂R^{e}, —N(R^{b})S(═O)₂NR^{f}R^{g}, and —NR^{f}R^{g};

each R^{d} is selected from the group consisting of optionally substituted —C₁-C₁₂ alkyl, optionally substituted —C₂-C₁₂ alkenyl, optionally substituted —C₂-C₁₂ alkynyl, optionally substituted —(CR^{b}₂)ₙ aryl, optionally substituted —(CR^{b}₂)ₙ cycloalkyl, optionally substituted —(CR^{b}₂)ₙ heterocycloalkyl, and —C(O)NR^{f}R^{g};

each R^{e} is selected from the group consisting of optionally substituted —C₁-C₁₂ alkyl, optionally substituted —C₂-C₁₂ alkenyl, optionally substituted —C₂-C₁₂ alkynyl, optionally substituted —(CR^{a}₂)ₙ aryl, optionally substituted —(CR^{a}₂)ₙ cycloalkyl, and optionally substituted —(CR^{a}₂)ₙ heterocycloalkyl;

R^{f} and R^{g} are each independently selected from the group consisting of hydrogen, optionally substituted —C₁-C₁₂ alkyl, optionally substituted —C₂-C₁₂ alkenyl, optionally substituted —C₂-C₁₂ alkynyl, optionally substituted —(CR^{b}₂)ₙ aryl, optionally substituted —(CR^{b}₂)ₙ cycloalkyl, and optionally substituted —(CR^{b}₂)ₙ heterocycloalkyl, or R^{f} and R^{g} may together form an optionally substituted heterocyclic ring, which may contain a second heterogroup selected from the group consisting of O, NR^{C}, and S, wherein said optionally substituted heterocyclic ring may be substituted with 0-4 substituents selected from the group consisting of optionally substituted —C₁-C₄ alkyl, —OR^{b}, oxo, cyano, —CF₃, optionally substituted phenyl, and —C(O)OR^{h};

each R^{h} is selected from the group consisting of optionally substituted —C₁-C₁₂ alkyl, optionally substituted —C₂-C₁₂ alkenyl, optionally substituted —C₂-C₁₂ alkynyl, optionally substituted —(CR^{b}₂)ₙ aryl, optionally substituted —(CR^{b}₂)ₙ cycloalkyl, and optionally substituted —(CR^{b}₂)ₙ heterocycloalkyl;

R⁵ is selected from the group consisting of —OH, optionally substituted —OC₁-C₆ alkyl, OC(O)R^{e}, —OC(O)OR^{h}, —F, —NHC(O)R^{e}, —NHS(═O)R^{e}, —NHS(═O)₂R^{e}, —NHC(═S)NH(R^{h}), and —NHC(O)NH(R^{h});

X is P(O)YR¹¹Y′R¹¹;

Y and Y′ are each independently selected from the group consisting of —O—, and —NR^{v}—; when Y and Y′ are —O—, R¹¹ attached to —O— is independently selected from the group consisting of —H, alkyl, optionally substituted aryl, optionally substituted heterocycloalkyl, optionally substituted CH₂-heterocycloakyl wherein the cyclic moiety contains a carbonate or thiocarbonate, optionally substituted -alkylaryl, —C(R^{z})₂OC(O)NR^{z}₂, —NR^{z}—C(O)—R^{y}, —C(R^{z})₂—OC(O)R^{y}, —C(R^{z})₂—O—C(O)OR^{y}, —C(R^{z})₂OC(O)SR^{y}, -alkyl-S—C(O)R^{y}, -alkyl-S—S-alkylhydroxy, and -alkyl-S—S—S-alkylhydroxy;

when Y and Y′ are —NR^{v}—, then R¹¹ attached to —NR^{v}— is independently selected from the group consisting of —H, —[C(R^{z})₂]_{q}—COOR^{y}, —C(R^{x})₂COOR^{y}, —[C(R^{z})₂]_{q}—C(O)SR^{y}, and -cycloalkylene-COOR^{y};

when Y is —O— and Y′ is NR^{v}, then R¹¹ attached to —O— is independently selected from the group consisting of —H, alkyl, optionally substituted aryl, optionally substituted heterocycloalkyl, optionally substituted CH₂-heterocycloakyl wherein the cyclic moiety contains a carbonate or thiocarbonate, optionally substituted -alkylaryl, —C(R^{z})₂OC(O)NR^{z}₂, —NR^{z}—C(O)—R^{y}, —C(R^{z})₂—OC(O)R^{y}, —C(R^{z})₂—O—C(O)OR^{y}, —C(R^{z})₂OC(O)SR^{y}, -alkyl-S—C(O)R^{y}, -alkyl-S—S-alkylhydroxy, and -alkyl-S—S—S-alkylhydroxy; and R¹¹ attached to —NR^{v}— is independently selected from the group consisting of H, —[C(R^{z})₂]_{q}—COOR^{y}, —C(R^{x})₂COOR^{y}, —[C(R^{z})₂]_{q}—C(O)SR^{y}, and -cycloalkylene-COOR^{y};

or when Y and Y′ are independently selected from —O— and NR^{v}, then together R¹¹ and R¹¹ are -alkyl-S—S-alkyl- to form a cyclic group, or together R¹¹ and R¹¹ are the group:

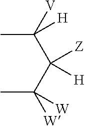

wherein:

V, W, and W′ are independently selected from the group consisting of hydrogen, optionally substituted alkyl, optionally substituted aralkyl, heterocycloalkyl, aryl, substituted aryl, heteroaryl, substituted heteroaryl, optionally substituted 1-alkenyl, and optionally substituted 1-alkynyl;

or together V and Z are connected via an additional 3-5 atoms to form a cyclic group containing 5-7 atoms, wherein 0-1 atoms are heteroatoms and the remaining atoms are carbon, substituted with hydroxy, acyloxy, alkylthiocarbonyloxy, alkoxycarbonyloxy, or aryloxycarbonyloxy attached to a carbon atom that is three atoms from both Y groups attached to the phosphorus;

or together V and Z are connected via an additional 3-5 atoms to form a cyclic group, wherein 0-1 atoms are heteroatoms and the remaining atoms are carbon, that is fused to an aryl group at the beta and gamma position to the Y attached to the phosphorus;

or together V and W are connected via an additional 3 carbon atoms to form an optionally substituted cyclic group containing 6 carbon atoms and substituted with one substituent selected from the group consisting of hydroxy, acyloxy, alkoxycarbonyloxy, alkylthiocarbonyloxy, and aryloxycarbonyloxy, attached to one of said carbon atoms that is three atoms from a Y attached to the phosphorus;

or together Z and W are connected via an additional 3-5 atoms to form a cyclic group, wherein 0-1 atoms are heteroatoms and the remaining atoms are carbon, and V must be aryl, substituted aryl, heteroaryl, or substituted heteroaryl;

or together W and W′ are connected via an additional 2-5 atoms to form a cyclic group, wherein 0-2 atoms are heteroatoms and the remaining atoms are carbon, and V must be aryl, substituted aryl, heteroaryl, or substituted heteroaryl;

Z is selected from the group consisting of —CHR^{z}OH, —CHR^{z}OC(O)R^{y}, —CHR^{z}OC(S)R^{y}, —CHR^{z}OC(S)OR^{y}, —CHR^{z}OC(O)SR^{y}, —CHR^{z}OCO₂R^{y}, —OR^{z}, —SR^{z}, —CHR^{z}N₃, —CH₂-aryl, —CH(aryl)OH, —CH(CH═CR^{z}₂)OH, —CH(C≡CR^{z})OH, —R^{z}, —NR^{z}₂, —OCOR^{y}, —OCO₂R^{y}, —SCOR^{y}, —SCO₂R^{y}, —NHCOR^{z}, —NHCO₂R^{y}, —CH₂NH-aryl, —(CH₂)q-OR^{z}, and —(CH₂)q-SR^{z};

q is an integer 2 or 3;

each R^{z} is selected from the group consisting of R^{y} and —H;

each R^{y} is selected from the group consisting of alkyl, aryl, heterocycloalkyl, and aralkyl;

each R^{x} is independently selected from the group consisting of —H, and alkyl, or together R^{x} and R^{x} form a cyclic alkyl group;

each R^{v} is selected from the group consisting of —H, lower alkyl, acyloxyalkyl, alkoxycarbonyloxyalkyl, and lower acyl;

and pharmaceutically acceptable salts and prodrugs thereof; and pharmaceutically acceptable salts of said prodrugs.

In some embodiments, the compound of Formula I has the following provisos:

a) when G is —O—, T is —CH₂—, R¹ and R² are each bromo, R³ is iso-propyl, R⁴ is hydrogen, and R⁵ is —OH, then X is not P(O)(OH)₂ or P(O)(OCH₂CH₃)₂;

b) V, Z, W, W′ are not all —H; and

c) when Z is —R^{z}, then at least one of V, W, and W′ is not —H, alkyl, aralkyl, or heterocycloalkyl;

d) when G is —O—, T is —(CH₂)₁₋₄—, R¹ and R² are independently halogen, alkyl, and cycloalkyl, R³ is alkyl, R⁴ is hydrogen, and R⁵ is —OH, then X is not —P(O)(OH)₂ or —P(O)(O-lower alkyl)₂; and

e) when G is —O—, R⁵ is —NHC(O)R^{e}, —NHS(═O)₁₋₂R^{e}, —NHC(S)NH(R^{b}), or —NHC(O)NH(R^{h}), T is —(CH₂)^{m}—, —CH═CH—, —O(CH₂)₁₋₂—, or —NH(CH₂)₁₋₂—, then X is not —P(O)(OH)₂ or —P(O)(OH)NH₂;

Preferably, the composition to be administered comprises one or more of the following:

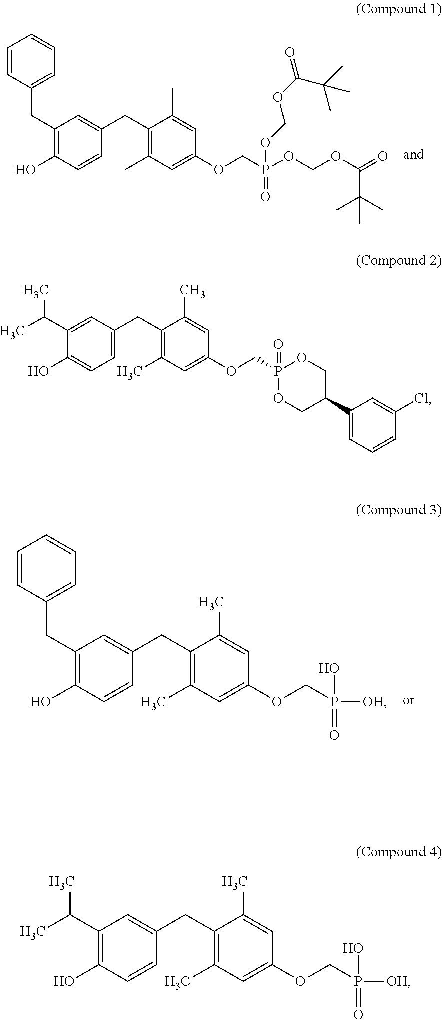

or pharmaceutically acceptable salts thereof.

The methods as described herein are effective in reducing or eliminating thyroid-related side effects and side effects related to suppression of the Hypothalmic-Thyroid-Pituitary axis (HPT axis) while maintaining the effectiveness of the compounds at the same or similar levels as seen under the standard daily dosing regimen. The methods described herein comprise the utilization of strategically placed dosing holidays, which surprisingly preserve the beneficial effect of the administered compounds while reducing suppression of the HPT axis. Such holidays may occur every other day during the dosing schedule, or more or less frequently. In some embodiments, dosing occurs daily for between 1 and 30 days, followed by a dosing holiday of between 1 and 30 days.

In some embodiments, serum levels of the drug substance are allowed to fall to sub-therapeutic levels prior to the administration of the next dose. In some other embodiments, serum levels of the drug substance are maintained within the therapeutic window in between doses. In some other embodiments, dosing is carried out daily with concurrent monitoring of the components of the HPT axis. In some further embodiments, dosing holidays occur whenever suppression of the HPT axis is directly observed.

### BRIEF DESCRIPTION OF THE DRAWINGS

FIG. 1 shows the effect of once-daily oral administration of Compound 2 on total plasma cholesterol (TPC) levels in beagle dogs (n=4 per group) over 14 days.

FIG. 2 shows the effect of once-daily oral administration of Compound 2 for 14 days followed by alternate day administration of Compound 2 for 14 days on total plasma cholesterol (TPC) levels in beagle dogs (n=4 per group).

FIG. 3 shows total T4/Thyroxine (tT4) levels (mean±SEM) in serum after once-daily oral administration of Compound 2 to beagle dogs (n=4/group).

FIG. 4 shows free T4/Thyroxine (fT4) levels (mean±SEM) in serum after once-daily oral administration of Compound 2 to beagle dogs (n=4/group).

FIG. 5 shows total triiodothyronine/T3 (tT3) levels (mean±SEM) in serum after once-daily oral administration of Compound 2 to beagle dogs (n=4/group).

FIG. 6 shows free triiodothyronine/T3 (fT3) levels (mean±SEM) in serum after once-daily oral administration of Compound 2 to beagle dogs (n=4/group).

FIG. 7 shows Thyroid Stimulating Hormone (TSH) levels (mean±SEM) in serum after once-daily oral administration of Compound 2 to beagle dogs (n=4/group).

FIG. 8 shows the effect of once-daily oral administration of Compound 2 for 14 days followed by alternate day administration of Compound 2 for 14 days on total T4 (tT4) levels in serum of beagle dogs (n=2/group).

FIG. 9 shows the effect of once-daily oral administration of Compound 2 for 14 days followed by alternate day administration of Compound 2 for 14 days on free T4 (fT4) levels in serum of beagle dogs (Cycle 2+Extension; n=2/group).

### DETAILED DESCRIPTION

The present disclosure provides methods for treating nonalcoholic fatty liver disease, non-alcoholic steatohepatitis, hyperlipidemia, dyslipidemia, hypertriglyceridemia, and other disorders linked to misregulation of the TRPβ pathway by administering TRPβ agonists. The methods of the present disclosure are further designed to prevent suppression of the HPT axis and the potential side effects that are associated with this suppression.

### Definitions

The term “mammal” is used in its usual biological sense. Thus, it specifically includes humans and non-human mammals such as dogs, cats, horses, donkeys, mules, cows, domestic buffaloes, camels, llamas, alpacas, bison, yaks, goats, sheep, pigs, elk, deer, domestic antelopes, and non-human primates as well as many other species.

“Subject” as used herein, means a human or a non-human mammal including but not limited to a dog, cat, horse, donkey, mule, cow, domestic buffalo, camel, llama, alpaca, bison, yak, goat, sheep, pig, elk, deer, domestic antelope, or a non-human primate selected for treatment or therapy.

“Subject suspected of having” means a subject exhibiting one or more clinical indicators of a disease or condition. In certain embodiments, the disease or condition is obesity. In certain embodiments, the disease or condition is hyperlipidemia. In certain embodiments, the disease or condition is hypercholesterolemia. In certain embodiments, the disease or condition is diabetes. In certain embodiments, the disease or condition is non-alcoholic fatty liver disease. In certain embodiments, the disease or condition is non-alcoholic steatohepatitis. In certain embodiments, the disease or condition is atherosclerosis. In certain embodiments, the disease or condition is cardiovascular disease. In certain embodiments, the disease or condition is hypothyroidism. In certain embodiments, the disease or condition is thyroid cancer.

“Subject in need thereof” means a subject identified as in need of a therapy or treatment.

A therapeutic effect relieves, to some extent, one or more of the symptoms of a disease or disorder, and includes curing the disease or disorder. “Curing” means that the symptoms of active disease are eliminated. However, certain long-term or permanent effects of the disease may exist even after a cure is obtained (such as extensive tissue damage).

“Treat,” “treatment,” or “treating,” as used herein refers to administering a pharmaceutical composition for prophylactic and/or therapeutic purposes. The term “prophylactic treatment” refers to treating a patient who does not yet have the relevant disease or disorder, but who is susceptible to, or otherwise at risk of, a particular disease or disorder, whereby the treatment reduces the likelihood that the patient will develop the disease or disorder. The term “therapeutic treatment” refers to administering treatment to a patient already having a disease or disorder.

“Preventing” or “prevention” refers to delaying or forestalling the onset, development or progression of a condition or disease for a period of time, including weeks, months, or years.

“Amelioration” means a lessening of severity of at least one indicator of a condition or disease. In certain embodiments, amelioration includes a delay or slowing in the progression of one or more indicators of a condition or disease. The severity of indicators may be determined by subjective or objective measures which are known to those skilled in the art.

“Modulation” means a perturbation of function or activity. In certain embodiments, modulation means an increase in gene expression. In certain embodiments, modulation means a decrease in gene expression. In certain embodiments, modulation means an increase or decrease in total serum levels of a specific protein. In certain embodiments, modulation means an increase or decrease in free serum levels of a specific protein. In certain embodiments, modulation means an increase or decrease in total serum levels of a specific non-protein factor. In certain embodiments, modulation means an increase or decrease in free serum levels of a specific non-protein factor. In certain embodiments, modulation means an increase or decrease in total bioavailability of a specific protein. In certain embodiments, modulation means an increase or decrease in total bioavailability of a specific non-protein factor.

“Administering” means providing a pharmaceutical agent or composition to a subject, and includes, but is not limited to, administering by a medical professional and self-administering.

Administration of the compounds disclosed herein or the pharmaceutically acceptable salts thereof can be via any of the accepted modes of administration for agents that serve similar utilities including, but not limited to, orally, subcutaneously, intravenously, intranasally, topically, transdermally, intraperitoneally, intramuscularly, intrapulmonarilly, vaginally, rectally, or intraocularly. Oral and parenteral administrations are customary in treating the indications that are the subject of the preferred embodiments.

“Parenteral administration,” means administration through injection or infusion. Parenteral administration includes, but is not limited to, subcutaneous administration, intravenous administration, intramuscular administration, intraarterial administration, and intracranial administration.

“Subcutaneous administration” means administration just below the skin.

“Intravenous administration” means administration into a vein.

“Intraarterial administration” means administration into an artery.

The term “agent” includes any substance, molecule, element, compound, entity, or a combination thereof. It includes, but is not limited to, e.g., protein, polypeptide, peptide or mimetic, small organic molecule, polysaccharide, polynucleotide, and the like. It can be a natural product, a synthetic compound, or a chemical compound, or a combination of two or more substances.

“Pharmaceutical agent” means a substance that provides a therapeutic effect when administered to a subject.

“Pharmaceutical composition” means a mixture of substances suitable for administering to an individual that includes a pharmaceutical agent. For example, a pharmaceutical composition may comprise a modified oligonucleotide and a sterile aqueous solution.

“Active pharmaceutical ingredient” means the substance in a pharmaceutical composition that provides a desired effect.

The term “pharmaceutically acceptable salt” refers to salts that retain the biological effectiveness and properties of the compounds with which they are associated and, which are not biologically or otherwise undesirable. In many cases, the compounds herein are capable of forming acid and/or base salts by virtue of the presence of phenol and/or phosphonate groups or groups similar thereto. One of ordinary skill in the art will be aware that the protonation state of any or all of these compounds may vary with pH and ionic character of the surrounding solution, and thus the present disclosure contemplates multiple charge states of each compound. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. Many such salts are known in the art, as described in WO 87/05297, Johnston et al., published Sep. 11, 1987 (incorporated by reference herein in its entirety).

“Solvate” refers to the compound formed by the interaction of a solvent and an EPI, a metabolite, or salt thereof. Suitable solvates are pharmaceutically acceptable solvates including hydrates.

The compounds useful as described above can be formulated into pharmaceutical compositions for use in treatment of these conditions. Standard pharmaceutical formulation techniques are used, such as those disclosed in Remington's The Science and Practice of Pharmacy, 21st Ed., Lippincott Williams & Wilkins (2005), incorporated herein by reference in its entirety. Accordingly, some embodiments include pharmaceutical compositions comprising: (a) a safe and therapeutically effective amount of a compound described herein, or pharmaceutically acceptable salts thereof; and (b) a pharmaceutically acceptable carrier, diluent, excipient or combination thereof.

The term “pharmaceutically acceptable carrier” or “pharmaceutically acceptable excipient” includes any and all solvents, diluents, emulsifiers, binders, buffers, dispersion media, coatings, antibacterial and antifungal agents, isotonic and absorption delaying agents and the like, or any other such compound as is known by those of skill in the art to be useful in preparing pharmaceutical formulations. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions. In addition, various adjuvants such as are commonly used in the art may be included. These and other such compounds are described in the literature, e.g., in the Merck Index, Merck & Company, Rahway, N.J. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press.

Some examples of substances, which can serve as pharmaceutically-acceptable carriers or components thereof, are sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerine, sorbitol, mannitol, and polyethylene glycol; alginic acid; emulsifiers, such as the TWEENS; wetting agents, such as sodium lauryl sulfate; coloring agents; flavoring agents; tableting agents, stabilizers; antioxidants; preservatives; pyrogen-free water; isotonic saline; and phosphate buffer solutions.

The choice of a pharmaceutically-acceptable carrier to be used in conjunction with the subject compound is determined by the way the compound is to be administered.

The compositions described herein are preferably provided in unit dosage form. As used herein, a “unit dosage form” is a composition containing an amount of a compound that is suitable for administration to a subject, in a single dose, according to good medical practice. The preparation of a single or unit dosage form however, does not imply that the dosage form is administered once per day or once per course of therapy. A unit dosage form may comprise a single daily dose or a fractional sub-dose wherein several unit dosage forms are to be administered over the course of a day in order to complete a daily dose. According to the present disclosure, a unit dosage form may be given more or less often that once daily, and may be administered more than once during a course of therapy. Such dosage forms may be administered in any manner consistent with their formulation, including orally, parenterally, and may be administered as an infusion over a period of time (e.g., from about 30 minutes to about 2-6 hours). While single administrations are specifically contemplated, the compositions administered according to the methods described herein may also be administered as a continuous infusion or via an implantable infusion pump.

The methods as described herein may utilize any of a variety of suitable forms for a variety of routes for administration, for example, for oral, nasal, rectal, topical (including transdermal), ocular, intracerebral, intracranial, intrathecal, intra-arterial, intravenous, intramuscular, or other parental routes of administration. The skilled artisan will appreciate that oral and nasal compositions include compositions that are administered by inhalation, and made using available methodologies. Depending upon the particular route of administration desired, a variety of pharmaceutically-acceptable carriers well-known in the art may be used. Pharmaceutically-acceptable carriers include, for example, solid or liquid fillers, diluents, hydrotropies, surface-active agents, and encapsulating substances. Optional pharmaceutically-active materials may be included, which do not substantially interfere with the activity of the compound. The amount of carrier employed in conjunction with the compound is sufficient to provide a practical quantity of material for administration per unit dose of the compound. Techniques and compositions for making dosage forms useful in the methods described herein are described in the following references, all incorporated by reference herein: Modern Pharmaceutics, 4th Ed., Chapters 9 and 10 (Banker & Rhodes, editors, 2002); Lieberman et al., Pharmaceutical Dosage Forms: Tablets (1989); and Ansel, Introduction to Pharmaceutical Dosage Forms 8th Edition (2004).

Various oral dosage forms can be used, including such solid forms as tablets, capsules, granules and bulk powders. Tablets can be compressed, tablet triturates, enteric-coated, sugar-coated, film-coated, or multiple-compressed, containing suitable binders, lubricants, diluents, disintegrating agents, coloring agents, flavoring agents, flow-inducing agents, and melting agents. Liquid oral dosage forms include aqueous solutions, emulsions, suspensions, solutions and/or suspensions reconstituted from non-effervescent granules, and effervescent preparations reconstituted from effervescent granules, containing suitable solvents, preservatives, emulsifying agents, suspending agents, diluents, sweeteners, melting agents, coloring agents and flavoring agents.

The pharmaceutically-acceptable carriers suitable for the preparation of unit dosage forms for peroral administration is well-known in the art. Tablets typically comprise conventional pharmaceutically-compatible adjuvants as inert diluents, such as calcium carbonate, sodium carbonate, mannitol, lactose and cellulose; binders such as starch, gelatin and sucrose; disintegrants such as starch, alginic acid and croscarmelose; lubricants such as magnesium stearate, stearic acid, microcrystalline cellulose, carboxymethyl cellulose, and talc. Tablets may also comprise solubilizers or emulsifiers, such as poloxamers, cremophor/Kolliphor®/Lutrol®, methylcellulose, hydroxypropylmethylcellulose, or others as are known in the art. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents disclosed above. The selection of carrier components depends on secondary considerations like taste, cost, and shelf stability, which can be readily made by a person skilled in the art.

Peroral (PO) compositions also include liquid solutions, emulsions, suspensions, and the like. The pharmaceutically-acceptable carriers suitable for preparation of such compositions are well known in the art. Typical components of carriers for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethyl cellulose, AVICEL RC-591, tragacanth and sodium alginate; typical wetting agents include lecithin and polysorbate 80; and typical preservatives include methyl paraben and sodium benzoate. Peroral liquid compositions may also contain one or more components such as sweeteners, flavoring agents and colorants disclosed above.

Such compositions may also be coated by conventional methods, typically with pH or time-dependent coatings, such that the subject compound is released in the gastrointestinal tract in the vicinity of the desired topical application, or at various times to extend the desired action. Such dosage forms typically include, but are not limited to, one or more of cellulose acetate phthalate, polyvinylacetate phthalate, hydroxypropyl methyl cellulose phthalate, ethyl cellulose, Eudragit coatings, waxes and shellac.

Compositions described herein may optionally include other drug actives.

Other compositions useful for attaining systemic delivery of the subject compounds include sublingual, buccal and nasal dosage forms. Such compositions typically comprise one or more of soluble filler substances such as sucrose, sorbitol and mannitol; and binders such as acacia, microcrystalline cellulose, carboxymethyl cellulose and hydroxypropyl methyl cellulose. Glidants, lubricants, sweeteners, colorants, antioxidants and flavoring agents disclosed above may also be included.

A liquid composition, which is formulated for topical ophthalmic use, is formulated such that it can be administered topically to the eye. The comfort may be maximized as much as possible, although sometimes formulation considerations (e.g. drug stability) may necessitate less than optimal comfort. In the case that comfort cannot be maximized, the liquid may be formulated such that the liquid is tolerable to the patient for topical ophthalmic use. Additionally, an ophthalmically acceptable liquid may either be packaged for single use, or contain a preservative to prevent contamination over multiple uses.

For ophthalmic application, solutions or medicaments are often prepared using a physiological saline solution as a major vehicle. Ophthalmic solutions may preferably be maintained at a comfortable pH with an appropriate buffer system. The formulations may also contain conventional, pharmaceutically acceptable preservatives, stabilizers and surfactants.

Preservatives that may be used in the pharmaceutical compositions disclosed herein include, but are not limited to, benzalkonium chloride, PHMB, chlorobutanol, thimerosal, phenylmercuric, acetate and phenylmercuric nitrate. A useful surfactant is, for example, Tween 80. Likewise, various useful vehicles may be used in the ophthalmic preparations disclosed herein. These vehicles include, but are not limited to, polyvinyl alcohol, povidone, hydroxypropyl methyl cellulose, poloxamers, carboxymethyl cellulose, hydroxyethyl cellulose and purified water.

Tonicity adjustors may be added as needed or convenient. They include, but are not limited to, salts, particularly sodium chloride, potassium chloride, mannitol and glycerin, or any other suitable ophthalmically acceptable tonicity adjustor.

Various buffers and means for adjusting pH may be used so long as the resulting preparation is ophthalmically acceptable. For many compositions, the pH will be between 4 and 9. Accordingly, buffers include acetate buffers, citrate buffers, phosphate buffers and borate buffers. Acids or bases may be used to adjust the pH of these formulations as needed.

Ophthalmically acceptable antioxidants include, but are not limited to, sodium metabisulfite, sodium thiosulfate, acetylcysteine, butylated hydroxyanisole and butylated hydroxytoluene.

Other excipient components, which may be included in the ophthalmic preparations, are chelating agents. A useful chelating agent is edetate disodium, although other chelating agents may also be used in place or in conjunction with it.

For topical use, including for transdermal administration, creams, ointments, gels, solutions or suspensions, etc., containing the compound disclosed herein are employed. Topical formulations may generally be comprised of a pharmaceutical carrier, co-solvent, emulsifier, penetration enhancer, preservative system, and emollient.

For intravenous administration, the compounds and compositions described herein may be dissolved or dispersed in a pharmaceutically acceptable diluent, such as a saline or dextrose solution. Suitable excipients may be included to achieve the desired pH, including but not limited to NaOH, sodium carbonate, sodium acetate, HCl, and citric acid. In various embodiments, the pH of the final composition ranges from 2 to 8, or preferably from 4 to 7. Antioxidant excipients may include sodium bisulfite, acetone sodium bisulfite, sodium formaldehyde, sulfoxylate, thiourea, and EDTA. Other non-limiting examples of suitable excipients found in the final intravenous composition may include sodium or potassium phosphates, citric acid, tartaric acid, gelatin, and carbohydrates such as dextrose, mannitol, and dextran. Further acceptable excipients are described in Powell, et al., Compendium of Excipients for Parenteral Formulations, PDA J Pharm Sci and Tech 1998, 52 238-311 and Nema et al., Excipients and Their Role in Approved Injectable Products: Current Usage and Future Directions, PDA J Pharm Sci and Tech 2011, 65 287-332, both of which are incorporated herein by reference in their entirety. Antimicrobial agents may also be included to achieve a bacteriostatic or fungistatic solution, including but not limited to phenylmercuric nitrate, thimerosal, benzethonium chloride, benzalkonium chloride, phenol, cresol, and chlorobutanol.

The compositions for intravenous administration may be provided to caregivers in the form of one more solids that are reconstituted with a suitable diluent such as sterile water, saline or dextrose in water shortly prior to administration. In other embodiments, the compositions are provided in solution ready to administer parenterally. In still other embodiments, the compositions are provided in a solution that is further diluted prior to administration. In embodiments that include administering a combination of a compound described herein and another agent, the combination may be provided to caregivers as a mixture, or the caregivers may mix the two agents prior to administration, or the two agents may be administered separately.

The actual unit dose of the active compounds described herein depends on the specific compound, and on the condition to be treated. In some embodiments, the dose may be from about 0.01 mg/kg to about 120 mg/kg or more of body weight, from about 0.05 mg/kg or less to about 70 mg/kg, from about 0.1 mg/kg to about 50 mg/kg of body weight, from about 1.0 mg/kg to about 10 mg/kg of body weight, from about 5.0 mg/kg to about 10 mg/kg of body weight, or from about 10.0 mg/kg to about 20.0 mg/kg of body weight. In some embodiments, the dose may be less than 100 mg/kg, 90 mg/kg, 80 mg/kg, 70 mg/kg, 60 mg/kg, 50 mg/kg, 40 mg/kg, 30 mg/kg, 25 mg/kg, 20 mg/kg, 10 mg/kg, 7.5 mg/kg, 6 mg/kg, 5 mg/kg, 4 mg/kg, 3 mg/kg, 2.5 mg/kg, 1 mg/kg, 0.5 mg/kg, 0.1 mg/kg, 0.05 mg/kg or 0.005 mg/kg of body weight. In some embodiments, the actual unit dose is 0.05, 0.07, 0.1, 0.3, 1.0, 3.0, 5.0, 10.0 or 25.0 mg/kg of body weight. Thus, for administration to a 70 kg person, the dosage range would be from about 0.1 mg to 70 mg, from about 1 mg to about 50 mg, from about 0.5 mg to about 10 mg, from about 1 mg to about 10 mg, from about 2.5 mg to about 30 mg, from about 35 mg or less to about 700 mg or more, from about 7 mg to about 600 mg, from about 10 mg to about 500 mg, from about 20 mg to about 300 mg, or from about 200 mg to about 2000 mg. In some embodiments, the actual unit dose is 5 mg. In some embodiments the actual unit dose is 10 mg. In some embodiments, the actual unit dose is 25 mg. In some embodiments, the actual unit dose is 250 mg or less. In some embodiments, the actual unit dose is 100 mg or less. In some embodiments, the actual unit dose is 70 mg or less. In some embodiments, the actual unit does is 5 mg.

“Loading dose,” as used herein refers to an initial dose of a compound which is higher than subsequent doses.

“Maintenance dose,” as used herein refers to a subsequent dose that follows a loading dose, and occurs later in time than a loading dose. One of ordinary skill in the art will be aware that the dosage form or mode of administration of a maintenance dose may be different from that used for the loading dose. In any of the embodiments disclosed herein, a maintenance dose may comprise administration of the unit dosage form on any dosing schedule contemplated herein, including but not limited to, monthly or multiple times per month, biweekly or multiple times each two weeks, weekly or multiple times per week, daily or multiple times per day. It is contemplated within the present disclosure that dosing holidays may be incorporated into the dosing period of the maintenance dose. Such dosing holidays may occur immediately after the administration of the loading dose or at any time during the period of administration of the maintenance dose. As used herein, the period of administration of the maintenance dose may be referred to as the “maintenance phase” of the treatment period.

“Mode of administration” as used herein refers to the means by which a compound is administered to a subject. As used herein, “mode of administration” comprises the dosage form (for example, a tablet, powder, dissolved liquid, suspension, emulsion, aerosol, etc.) and mechanism by which the dosage form is applied to the subject (for example, by injection, such as subcutaneously, intramuscularly, intraperitoneally, intravenously, or intraarterially; topically, such as by cream, lotion, or patch; orally, such as by a pill, dissolved liquid, oral suspension, buccal film, or mouthrinse; nasally, such as by a nasal aerosol, powder, or spray; or ocularly, such as by an eye drop). As used herein, “mode of administration” also comprises the dose, dose amount, and dosing schedule by which a compound is administered to a subject.

In some embodiments, the mode of administration comprises administering a loading dose followed by a maintenance dose. In some embodiments, the loading dose is 300 mg or less; 250 mg or less, 200 mg or less, 150 mg or less, or 100 mg or less. In some embodiments, the maintenance dose is 300 mg or less; 200 mg or less, 100 mg or less, 50 mg or less, 40 mg or less, 25 mg or less, 10 mg or less, 5 mg or less, or 1 mg or less.

In some embodiments the loading dose is administered over a period of one day. In some embodiments the loading dose is administered over a period of 2 days. In some embodiments the loading dose is administered over a period of 3 days. In some embodiments the loading dose is administered over a period of 4 days. In some embodiments the loading dose is administered over a period of 5, 6 or 7 days. In some embodiments, the loading dose is administered over a period of 8-14 days or fewer. In some embodiments, the loading dose is administered over a period of 14 days.

As used herein, “duration of the treatment” refers to the time commencing with administration of the first dose and concluding with the administration of the final dose, such length of time being determined by one of ordinary skill in the art of treating diseases implicating TRPβ, including but not limited to hyperlipidemia, hypercholesterolemia, NASH, and NAFLD with reference to the symptoms and health of the subject being treated therefor.

As used herein, “dosing holiday” refers to a period of 24 hours or more during which either no dose is administered to the subject, or a reduced dose is administered to the subject. As used herein, “reduced dose” refers to a dose that is less than the total daily dose to be administered to a subject.

As used herein, the “Hypothalamic-Pituitary-Thyroid Axis” or “HPT Axis” refers to the set of neuroendocrine pathways, signals, and molecules responsible for the regulation of metabolism. As used herein, “HPT Axis” further refers to any molecule involved in the regulation, modification, or response to thyroid hormone. Representative components of the HPT axis include Triiodothyronine (T3), Thyroxine (T4), iodothyronines, thyrotropin-releasing hormone (TRH), and thyroid-stimulating hormone (TSH).

The term “alkyl” refers to a straight or branched or cyclic chain hydrocarbon radical with only single carbon-carbon bonds. Representative examples include methyl, ethyl, propyl, isopropyl, cyclopropyl, butyl, isobutyl, tert-butyl, cyclobutyl, pentyl, cyclopentyl, hexyl, and cyclohexyl, all of which may be optionally substituted. Alkyl groups are C₁-C₁₂.

The term “aryl” refers to aromatic groups which have 5-14 ring atoms and at least one ring having a conjugated pi electron system and includes carbocyclic aryl, heterocyclic aryl and biaryl groups, all of which may be optionally substituted.

Carbocyclic aryl groups are groups which have 6-14 ring atoms wherein the ring atoms on the aromatic ring are carbon atoms. Carbocyclic aryl groups include monocyclic carbocyclic aryl groups and polycyclic or fused compounds such as optionally substituted naphthyl groups.

Heterocyclic aryl or heteroaryl groups are groups which have 5-14 ring atoms wherein 1 to 4 heteroatoms are ring atoms in the aromatic ring and the remainder of the ring atoms being carbon atoms. Suitable heteroatoms include oxygen, sulfur, nitrogen, and selenium. Suitable heteroaryl groups include furanyl, thienyl, pyridyl, pyrrolyl, N-lower alkyl pyrrolyl, pyridyl-N-oxide, pyrimidyl, pyrazinyl, imidazolyl, and the like, all optionally substituted.

The term “biaryl” represents aryl groups which have 5-14 atoms containing more than one aromatic ring including both fused ring systems and aryl groups substituted with other aryl groups. Such groups may be optionally substituted. Suitable biaryl groups include naphthyl and biphenyl.

The term “optionally substituted” or “substituted” includes groups substituted by one to six substituents, independently selected from lower alkyl, lower aryl, lower aralkyl, lower cyclic alkyl, lower heterocycloalkyl, hydroxy, lower alkoxy, lower aryloxy, perhaloalkoxy, aralkoxy, lower heteroaryl, lower heteroaryloxy, lower heteroarylalkyl, lower heteroaralkoxy, azido, amino, halo, lower alkylthio, oxo, lower acylalkyl, lower carboxy esters, carboxyl, -carboxamido, nitro, lower acyloxy, lower aminoalkyl, lower alkylaminoaryl, lower alkylaryl, lower alkylaminoalkyl, lower alkoxyaryl, lower arylamino, lower aralkylamino, sulfonyl, lower-carboxamidoalkylaryl, lower-carboxamidoaryl, lower hydroxyalkyl, lower haloalkyl, lower alkylaminoalkylcarboxy-, lower aminocarboxamidoalkyl-, cyano, lower alkoxyalkyl, lower perhaloalkyl, and lower arylalkyloxyalkyl.

“Substituted aryl” and “substituted heteroaryl” refers to aryl and heteroaryl groups substituted with 1-3 substituents. These substituents are selected from the group consisting of lower alkyl, lower alkoxy, lower perhaloalkyl, halo, hydroxy, and amino.

The term “-aralkyl” refers to an alkylene group substituted with an aryl group. Suitable aralkyl groups include benzyl, picolyl, and the like, and may be optionally substituted. “Heteroarylalkyl” refers to an alkylene group substituted with a heteroaryl group.

The term “alkylaryl-” refers to an aryl group substituted with an alkyl group. “Lower alkylaryl-” refers to such groups where alkyl is lower alkyl.

The term “lower” referred to herein in connection with organic radicals or compounds respectively defines such as with up to and including 10, in one aspect up to and including 6, and in another aspect one to four carbon atoms. Such groups may be straight chain, branched, or cyclic.

The term “cyclic alkyl” or “cycloalkyl” refers to alkyl groups that are cyclic of 3 to 10 carbon atoms, and in one aspect are 3 to 6 carbon atoms Suitable cyclic groups include norbornyl and cyclopropyl. Such groups may be substituted.

The term “heterocyclic”, “heterocyclic alkyl” or “heterocycloalkyl” refer to cyclic groups of 3 to 10 atoms, and in one aspect are 3 to 6 atoms, containing at least one heteroatom, in a further aspect are 1 to 3 heteroatoms. Suitable heteroatoms include oxygen, sulfur, and nitrogen. Heterocyclic groups may be attached through a nitrogen or through a carbon atom in the ring. The heterocyclic alkyl groups include unsaturated cyclic, fused cyclic and spirocyclic groups. Suitable heterocyclic groups include pyrrolidinyl, morpholino, morpholinoethyl, and pyridyl.

The terms “arylamino” (a), and “aralkylamino” (b), respectively, refer to the group —NRR′ wherein respectively, (a) R is aryl and R′ is hydrogen, alkyl, aralkyl, heterocycloalkyl, or aryl, and (b) R is aralkyl and R′ is hydrogen, aralkyl, aryl, alkyl or heterocycloalkyl.

The term “acyl” refers to —C(O)R where R is alkyl, heterocycloalkyl, or aryl.

The term “carboxy esters” refers to —C(O)OR where R is alkyl, aryl, aralkyl, cyclic alkyl, or heterocycloalkyl, all optionally substituted.

The term “carboxyl” refers to —C(O)OH.

The term “oxo” refers to ═O in an alkyl or heterocycloalkyl group.

The term “amino” refers to —NRR′ where R and R′ are independently selected from hydrogen, alkyl, aryl, aralkyl and heterocycloalkyl, all except H are optionally substituted; and R and R′ can form a cyclic ring system.

The term “-carboxylamido” refers to —CONR₂ where each R is independently hydrogen or alkyl.

The term “-sulphonylamido” or “-sulfonylamido” refers to —S(═O)₂NR₂ where each R is independently hydrogen or alkyl.

The term “halogen” or “halo” refers to —F, —Cl, —Br and —I.

The term “alkylaminoalkylcarboxy” refers to the group alkyl-NR-alk-C(O)—O— where “alk” is an alkylene group, and R is a H or lower alkyl.

The term “sulphonyl” or “sulfonyl” refers to —SO₂R, where R is H, alkyl, aryl, aralkyl, or heterocycloalkyl.

The term “sulphonate” or “sulfonate” refers to —SO₂OR, where R is —H, alkyl, aryl, aralkyl, or heterocycloalkyl.

The term “alkenyl” refers to unsaturated groups which have 2 to 12 atoms and contain at least one carbon-carbon double bond and includes straight-chain, branched-chain and cyclic groups. Alkenyl groups may be optionally substituted. Suitable alkenyl groups include allyl. “1-alkenyl” refers to alkenyl groups where the double bond is between the first and second carbon atom. If the 1-alkenyl group is attached to another group, e.g., it is a W substituent attached to the cyclic phosphonate, it is attached at the first carbon.

The term “alkynyl” refers to unsaturated groups which have 2 to 12 atoms and contain at least one carbon-carbon triple bond and includes straight-chain, branched-chain and cyclic groups. Alkynyl groups may be optionally substituted. Suitable alkynyl groups include ethynyl. “1-alkynyl” refers to alkynyl groups where the triple bond is between the first and second carbon atom. If the 1-alkynyl group is attached to another group, e.g., it is a W substituent attached to the cyclic phosphonate, it is attached at the first carbon.

The term “alkylene” refers to a divalent straight chain, branched chain or cyclic saturated aliphatic group. In one aspect the alkylene group contains up to and including 10 atoms. In another aspect the alkylene chain contains up to and including 6 atoms. In a further aspect the alkylene groups contains up to and including 4 atoms. The alkylene group can be either straight, branched or cyclic.

The term “acyloxy” refers to the ester group —O—C(O)R, where R is H, alkyl, alkenyl, alkynyl, aryl, aralkyl, or heterocycloalkyl.

The term “aminoalkyl-” refers to the group NR₂-alk- wherein “alk” is an alkylene group and R is selected from —H, alkyl, aryl, aralkyl, and heterocycloalkyl.

The term “alkylaminoalkyl-” refers to the group alkyl-NR-alk- wherein each “alk” is an independently selected alkylene, and R is H or lower alkyl. “Lower alkylaminoalkyl-” refers to groups where the alkyl and the alkylene group is lower alkyl and alkylene, respectively.

The term “arylaminoalkyl-” refers to the group aryl-NR-alk- wherein “alk” is an alkylene group and R is —H, alkyl, aryl, aralkyl, or heterocycloalkyl. In “lower arylaminoalkyl-”, the alkylene group is lower alkylene.

The term “alkylaminoaryl-” refers to the group alkyl-NR-aryl- wherein “aryl” is a divalent group and R is —H, alkyl, aralkyl, or heterocycloalkyl. In “lower alkylaminoaryl-”, the alkyl group is lower alkyl.

The term “alkoxyaryl-” refers to an aryl group substituted with an alkyloxy group. In “lower alkyloxyaryl-”, the alkyl group is lower alkyl.

The term “aryloxyalkyl-” refers to an alkyl group substituted with an aryloxy group.

The term “aralkyloxyalkyl-” refers to the group aryl-alk-O-alk- wherein “alk-” is an alkylene group. “Lower aralkyloxyalkyl-” refers to such groups where the alkylene groups are lower alkylene.

The term “alkoxy-” or “alkyloxy-” refers to the group alkyl-O—.

The term “alkoxyalkyl-” or “alkyloxyalkyl-” refer to the group alkyl-O-alk- wherein “alk” is an alkylene group. In “lower alkoxyalkyl-”, each alkyl and alkylene is lower alkyl and alkylene, respectively.

The terms “alkylthio-” and “alkylthio-” refer to the group alkyl-S—.

The term “alkylthioalkyl-” refers to the group alkyl-S-alk- wherein “alk” is an alkylene group. In “lower alkylthioalkyl-” each alkyl and alkylene is lower alkyl and alkylene, respectively.

The term “alkoxycarbonyloxy-” refers to alkyl-O—C(O)—O—.

The term “aryloxycarbonyloxy-” refers to aryl-O—C(O)—O—.

The term “alkylthiocarbonyloxy-” refers to alkyl-S—C(O)—O—.

The term “amido” refers to the NR₂ group next to an acyl or sulfonyl group as in NR₂—C(O)—, RC(O)—NR¹—, NR₂—S(═O)₂— and RS(═O)₂—NR¹, where R and R′ include —H, alkyl, aryl, aralkyl, and heterocycloalkyl.

The term “carboxamido” refer to NR₂—C(O)— and RC(O)—NR¹—, where R and R′ include —H, alkyl, aryl, aralkyl, and heterocycloalkyl. The term does not include urea, —NR—C(O)—NR—.

The terms “sulphonamido” or “sulfonamido” refer to NR₂—S(═O)₂— and RS(═O)₂—NR¹—, where R and R′ include —H, alkyl, aryl, aralkyl, and heterocycloalkyl. The term does not include sulfonylurea, —NR—S(—O)₂—NR—.

The term “carboxamidoalkylaryl” and “carboxamidoaryl” refers to an aryl-alk-NR¹—C(O), and ar-NR¹—C(O)-alk-, respectively where “ar” is aryl, “alk” is alkylene, R¹ and R include H, alkyl, aryl, aralkyl, and heterocycloalkyl.

The term “sulfonamidoalkylaryl” and “sulfonamidoaryl” refers to an aryl-alk-NR¹—S(═O)₂—, and ar-NR¹—S(—O)₂—, respectively where “ar” is aryl, “alk” is alkylene, R¹ and R include —H, alkyl, aryl, aralkyl, and heterocycloalkyl.

The term “hydroxyalkyl” refers to an alkyl group substituted with one —OH.

The term “haloalkyl” refers to an alkyl group substituted with one halo.

The term “cyano” refers to —C≡N.

The term “nitro” refers to —NO₂.

The term “acylalkyl” refers to an alkyl-C(O)-alk-, where “alk” is alkylene.

The term “aminocarboxamidoalkyl-” refers to the group NR₂—C(O)—N(R)-alk- wherein R is an alkyl group or H and “alk” is an alkylene group. “Lower aminocarboxamidoalkyl-” refers to such groups wherein “alk” is lower alkylene.

The term “heteroarylalkyl” refers to an alkylene group substituted with a heteroaryl group.

The term “perhalo” refers to groups wherein every C—H bond has been replaced with a C-halo bond on an aliphatic or aryl group. Suitable perhaloalkyl groups include —CF₃ and —CFCl₂.

The term “carboxylic acid moiety” refers to a compound having a carboxylic acid group (—COOH), and salts thereof, a carboxylic acid ester, or a carboxylic acid surrogate. Suitable carboxylic acid surrogates include a tetrazole group, a hydroxamic acid group, a thiazolidinedione group, an acylsulfonamide group, and a 6-azauracil; and prodrugs thereof. Phosphonic acids and prodrugs thereof are not within the scope of carboxylic acid surrogates.

TABLE 1
List of Abbreviations and Terms

| | |
|---|---|
| ALP | Alkaline Phosphatase |
| ALT (SGPT) | Alanine Aminotransferase |
| AST (SGOT) | Aspartate Aminotransferase |
| BMI | Body Mass Index |
| BNP | B-type natriuretic peptide |
| BUN | Blood Urea Nitrogen |
| CAD | Coronary Artery Disease |
| CHD | Coronary Heart Disease |
| CHF | Congestive Heart Failure |
| CK | Creatine Kinase |
| CK-MB | Creatine Kinase MB Isoenzyme |
| CRP | C-reactive Protein |
| cTnI | Cardiac Troponin I |
| DB | Direct Bilirubin |
| DBP | Diastolic Blood Pressure |
| EKG | Electrocardiogram |
| FSG | Fasting Serum Glucose |
| FT3 | Free 3,3′,5-Triiodo-L-thyronine |
| FT4 | Free Thyroxine |
| HDL-C | High-Density Lipoprotein Cholesterol |
| LDL-C | Low-Density Lipoprotein Cholesterol |
| LFT | Liver Function Test |
| Lp(a) | Lipoprotein (a) |
| MCHC | Mean Corpuscular Hemoglobin Concentration |
| NASH | Nonalcoholic Steatohepatitis |
| PO | By Mouth/Orally |
| SBP | Systolic Blood Pressure |
| T3 | 3,3′,5-Triiodo-L-thyronine |
| TT3 | Total 3,3′,5-Triiodo-L-thyronine |
| T4 | Thyroxine |
| TT4 | Total Thyroxine |
| TB | Total Bilirubin |
| TC | Total Cholesterol |
| TG | Triglycerides |
| TR | Thyroid Hormone Receptors |
| TRβ | Thyroid Hormone Receptor - β isoform |
| TSH | Thyroid Stimulating Hormone |
| U/L | Units/liter |
| ULN | Upper Limit of Normal |
| VLDL-C | Very Low-Density Lipoprotein Cholesterol |
| WBC | White Blood Cell |

Table 1 provides definitions for common abbreviations in the art as used in the embodiments and examples described herein.

As used herein, “suppression of the HPT axis” refers to reductions in the circulating levels of any element of the HPT axis, especially Triiodothyronine (T3), Thyroxine (T4), iodothyronines, thyrotropin-releasing hormone (TRH), and thyroid-stimulating hormone (TSH), either individually, in any combination, or in the aggregate. In some embodiments, suppression of the HPT axis comprises a reduction in circulating serum levels of Triiodothyronine (T3), Thyroxine (T4), iodothyronines, thyrotropin-releasing hormone (TRH), or thyroid-stimulating hormone (TSH) by at least 5%. In some embodiments, suppression of the HPT axis comprises a reduction in circulating serum levels of Triiodothyronine (T3), Thyroxine (T4), iodothyronines, thyrotropin-releasing hormone (TRH), or thyroid-stimulating hormone (TSH) by at least 10%. In some embodiments, suppression of the HPT axis comprises a reduction in circulating serum levels of Triiodothyronine (T3), Thyroxine (T4), iodothyronines, thyrotropin-releasing hormone (TRH), or thyroid-stimulating hormone (TSH) by at least 20%. In some embodiments, suppression of the HPT axis comprises a reduction in circulating serum levels of Triiodothyronine (T3), Thyroxine (T4), iodothyronines, thyrotropin-releasing hormone (TRH), or thyroid-stimulating hormone (TSH) by at least 30%, 40%, 50%, or 60%. In some embodiments, suppression of the HPT axis comprises a reduction in circulating serum levels of Triiodothyronine (T3), Thyroxine (T4), iodothyronines, thyrotropin-releasing hormone (TRH), or thyroid-stimulating hormone (TSH) by more than 60%.

In some embodiments, suppression of the HPT axis comprises a reduction in levels of free Triiodothyronine (fT3), or free Thyroxine (fT4), by at least 5%. In some embodiments, suppression of the HPT axis comprises a reduction in levels of free Triiodothyronine (fT3), or free Thyroxine (fT4) by at least 10%. In some embodiments, suppression of the HPT axis comprises a reduction in levels of free Triiodothyronine (fT3), or free Thyroxine (fT4) by at least 20%. In some embodiments, suppression of the HPT axis comprises a reduction in levels of free Triiodothyronine (fT3), or free Thyroxine (fT4) by at least 30%, 40%, 50%, or 60%. In some embodiments, suppression of the HPT axis comprises a reduction in levels of free Triiodothyronine (fT3), or free Thyroxine (fT4) by more than 60%.

In some embodiments, suppression of the HPT axis comprises a reduction in levels of total Triiodothyronine (tT3), or total Thyroxine (tT4), by at least 5%. In some embodiments, suppression of the HPT axis comprises a reduction in levels of total Triiodothyronine (tT3), or total Thyroxine (tT4) by at least 10%. In some embodiments, suppression of the HPT axis comprises a reduction in levels of total Triiodothyronine (tT3), or total Thyroxine (tT4) by at least 20%. In some embodiments, suppression of the HPT axis comprises a reduction in levels of total Triiodothyronine (tT3), or total Thyroxine (tT4) by at least 30%, 40%, 50%, or 60%. In some embodiments, suppression of the HPT axis comprises a reduction in levels of total Triiodothyronine (tT3), or total Thyroxine (tT4) by more than 60%.

As contemplated herein, relief of thyroid-associated side effects comprises an effect of a treatment method wherein the level of suppression of the HPT axis is less than the level of suppression seen in daily dosing at between about 10 and about 40 mg/day per subject. In some embodiments, relief of thyroid-associated side effects comprises an effect of a treatment method wherein the level of suppression of the HPT axis is less than the level of suppression seen in daily dosing at 40 mg/day, 30 mg/day, 20 mg/day, 15 mg/day, 10 mg/day, 5 mg/day, or 2.5 mg/day for an individual subject. In some embodiments, relief of thyroid-associated side effects comprises an effect of a treatment method wherein the level of suppression of the HPT axis is less than the level of suppression seen in daily dosing at 2.5-35 mg/day, 2.5-10 mg/day, 5-15 mg/day, 5 mg/day, or 10 mg/day for an individual subject.

The present disclosure provides methods of administering compositions comprising one or more compounds of Formula I:

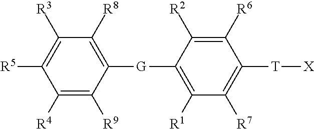

wherein:

G is selected from the group consisting of —O—, —S—, —S(═O)—, —S(═O)₂—, —Se—, —CH₂—, —CF₂—, —CHF—, —C(O)—, —CH(OH)—, —CH(C₁-C₄ alkyl)-, —CH(C₁-C₄ alkoxy)-, —C(—CH₂)—, —NH—, and —N(C₁-C₄ alkyl)-;

T is selected from the group consisting of —(CR^{a}₂)ₖ—, —CR^{b}═CR^{b}—(CR^{a}₂)ₙ—, —(CR^{a}₂)ₙ—CR^{b}═CR^{b}—, —(CR^{a}₂)—CR^{b}═CR^{b}—(CR^{a}₂), —O(CR^{b}₂)(CR^{a}₂)ₙ—, —S(CR^{b}₂)(CR^{a}₂)ₙ—, N(R^{C})(CR^{b}₂)(CR^{a}₂)ₙ—, N(R^{b})C(O)(CR^{a}₂)ₙ, —C(O)(CR^{a}₂)^{m}, (CR^{a}₂)ₘC(O)—, —(CR^{a}₂)C(O)(CR^{a}₂)ₙ, —(CR^{a}₂)ₙC(O)(CR^{a}₂)—, and —C(O)NH(CR^{b}₂)(CR^{a}₂)ₚ;

k is an integer from 1-4;

m is an integer from 0-3;

n is an integer from 0-2;

p is an integer from 0-1;

Each R^{a} is independently selected from the group consisting of hydrogen, optionally substituted —C₁-C₄ alkyl, halogen, —OH, optionally substituted —O—C₁-C₄ alkyl, —OCF₃, optionally substituted —S—C₁-C₄ alkyl, —NR^{b}R^{c}, optionally substituted —C₂-C₄ alkenyl, and optionally substituted —C₂-C₄ alkynyl; with the proviso that when one R^{a} is attached to C through an O, S, or N atom, then the other R^{a} attached to the same C is a hydrogen, or attached via a carbon atom;

each R^{b} is independently selected from the group consisting of hydrogen and optionally substituted —C₁-C₄ alkyl;

each R^{c} is independently selected from the group consisting of hydrogen and optionally substituted —C₁-C₄ alkyl, optionally substituted —C(O)—C₁-C₄ alkyl, and —C(O)H;

R¹, and R² are each independently selected from the group consisting of halogen, optionally substituted —C₁-C₄ alkyl, optionally substituted —S—C₁-C₃ alkyl, optionally substituted —C₂-C₄ alkenyl, optionally substituted —C₂-C₄ alkynyl, —CF₃, —OCF₃, optionally substituted-O—C₁-C₃ alkyl, and cyano;

R⁶, R⁷, R⁸, and R⁹ are each independently selected from the group consisting of hydrogen, halogen, optionally substituted —C₁-C₄ alkyl, optionally substituted —S—C₁-C₃ alkyl, optionally substituted —C₂-C₄ alkenyl, optionally substituted —C₂-C₄ alkynyl, —CF₃, —OCF₃, optionally substituted-O—C₁-C₃ alkyl, and cyano; or R⁶ and T are taken together along with the carbons they are attached to form a ring of 5 to 6 atoms including 0 to 2 heteroatoms independently selected from —NRⁱ—, —O—, and —S—, with the proviso that when there are 2 heteroatoms in the ring and both heteroatoms are different than nitrogen then both heteroatoms have to be separated by at least one carbon atom; and X is attached to this ring by a direct bond to a ring carbon, or by —(CR^{a}₂)— or —C(O)— bonded to a ring carbon or a ring nitrogen;

Rⁱ is selected from the group consisting of hydrogen, —C(O)C₁-C₄ alkyl, —C₁-C₄ alkyl, and —C₁-C₄-aryl;

R³ and R⁴ are independently selected from the group consisting of hydrogen, halogen, —CF₃, —OCF₃, cyano, optionally substituted —C₁-C₁₂ alkyl, optionally substituted —C₂-C₁₂ alkenyl, optionally substituted —C₂-C₁₂ alkynyl, —SR^{d}, —S(═O)R^{e}, —S(═O)₂R^{e}, —S(═O)₂NR^{f}R^{g}, —C(O)OR^{h}, —C(O)R^{e}, N(R^{b})C(O)NR^{f}R^{g}, —N(R^{b})S(═O)₂R^{e}, —N(R^{b})S(═O)₂NR^{f}R^{g}, and —NR^{f}R^{g};

each R^{d} is selected from the group consisting of optionally substituted —C₁-C₁₂ alkyl, optionally substituted —C₂-C₁₂ alkenyl, optionally substituted —C₂-C₁₂ alkynyl, optionally substituted —(CR^{b}₂)ₙ aryl, optionally substituted —(CR^{b}₂)ₙ cycloalkyl, optionally substituted —(CR^{b}₂)ₙ heterocycloalkyl, and —C(O)NR^{f}R^{g};

each R^{e} is selected from the group consisting of optionally substituted —C₁-C₁₂ alkyl, optionally substituted —C₂-C₁₂ alkenyl, optionally substituted —C₂-C₁₂ alkynyl, optionally substituted —(CR^{a}₂)ₙ aryl, optionally substituted —(CR^{a}₂)ₙ cycloalkyl, and optionally substituted —(CR^{a}₂)ₙ heterocycloalkyl;

R^{f} and R^{g} are each independently selected from the group consisting of hydrogen, optionally substituted —C₁-C₁₂ alkyl, optionally substituted —C₂-C₁₂ alkenyl, optionally substituted —C₂-C₁₂ alkynyl, optionally substituted —(CR^{b}₂)ₙ aryl, optionally substituted —(CR^{b}₂)ₙ cycloalkyl, and optionally substituted —(CR^{b}₂)ₙ heterocycloalkyl, or R^{f} and R^{g} may together form an optionally substituted heterocyclic ring, which may contain a second heterogroup selected from the group consisting of O, NR^{c}, and S, wherein said optionally substituted heterocyclic ring may be substituted with 0-4 substituents selected from the group consisting of optionally substituted —C₁-C₄ alkyl, —OR^{b}, oxo, cyano, —CF₃, optionally substituted phenyl, and —C(O)OR^{h};

each R^{h} is selected from the group consisting of optionally substituted —C₁-C₁₂ alkyl, optionally substituted —C₂-C₁₂ alkenyl, optionally substituted —C₂-C₁₂ alkynyl, optionally substituted —(CR^{b}₂)ₙ aryl, optionally substituted —(CR^{b}₂)ₙ cycloalkyl, and optionally substituted —(CR^{b}₂)ₙ heterocycloalkyl;

R⁵ is selected from the group consisting of —OH, optionally substituted —OC₁-C₆ alkyl, OC(O)R^{e}, —OC(O)OR^{h}, —F, —NHC(O)R^{e}, —NHS(═O)R^{e}, —NHS(═O)₂R^{e}, —NHC(═S)NH(R^{h}), and —NHC(O)NH(R^{h});

X is P(O)YR¹¹Y′R¹¹;

Y and Y′ are each independently selected from the group consisting of —O—, and —NR^{v}—; when Y and Y′ are —O—, R¹¹ attached to —O— is independently selected from the group consisting of —H, alkyl, optionally substituted aryl, optionally substituted heterocycloalkyl, optionally substituted CH₂-heterocycloakyl wherein the cyclic moiety contains a carbonate or thiocarbonate, optionally substituted -alkylaryl, —C(R^{z})₂OC(O)NR^{z}₂, —NR^{z}—C(O)—R^{y}, —C(R^{z})₂—OC(O)R^{y}, —C(R^{z})₂—O—C(O)OR^{y}, —C(R^{z})₂OC(O)SR^{y}, -alkyl-S—C(O)R^{y}, -alkyl-S—S-alkylhydroxy, and -alkyl-S—S—S-alkylhydroxy;

when Y and Y′ are —NR^{v}—, then R¹¹ attached to —NR^{v}— is independently selected from the group consisting of —H, —[C(R^{z})₂]_{q}—COOR^{y}, —C(R^{x})₂COOR^{y}, —[C(R^{z})₂]_{q}—C(O)SR^{y}, and -cycloalkylene-COOR^{y};

when Y is —O— and Y′ is NR^{v}, then R¹¹ attached to —O— is independently selected from the group consisting of —H, alkyl, optionally substituted aryl, optionally substituted heterocycloalkyl, optionally substituted CH₂-heterocycloakyl wherein the cyclic moiety contains a carbonate or thiocarbonate, optionally substituted -alkylaryl, —C(R^{z})₂OC(O)NR^{z}₂, —NR^{z}—C(O)—R^{y}, —C(R^{z})₂—OC(O)R^{y}, —C(R^{z})₂—O—C(O)OR^{y}, —C(R^{z})₂OC(O)SR^{y}, -alkyl-S—C(O)R^{y}, -alkyl-S—S-alkylhydroxy, and -alkyl-S—S—S-alkylhydroxy; and R¹ attached to —NR^{v}— is independently selected from the group consisting of H, —[C(R^{z})₂]_{q}—COOR^{y}, —C(R^{x})₂COOR^{y}, —[C(R^{z})₂]_{q}—C(O)SR^{y}, and -cycloalkylene-COOR^{y};

or when Y and Y′ are independently selected from —O— and NR^{v}, then together R¹¹ and R¹¹ are -alkyl-S—S-alkyl- to form a cyclic group, or together R¹¹ and R¹¹ are the group:

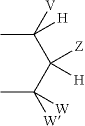

wherein:

V, W, and W′ are independently selected from the group consisting of hydrogen, optionally substituted alkyl, optionally substituted aralkyl, heterocycloalkyl, aryl, substituted aryl, heteroaryl, substituted heteroaryl, optionally substituted 1-alkenyl, and optionally substituted 1-alkynyl;

or together V and Z are connected via an additional 3-5 atoms to form a cyclic group containing 5-7 atoms, wherein 0-1 atoms are heteroatoms and the remaining atoms are carbon, substituted with hydroxy, acyloxy, alkylthiocarbonyloxy, alkoxycarbonyloxy, or aryloxycarbonyloxy attached to a carbon atom that is three atoms from both Y groups attached to the phosphorus; or

or together V and Z are connected via an additional 3-5 atoms to form a cyclic group, wherein 0-1 atoms are heteroatoms and the remaining atoms are carbon, that is fused to an aryl group at the beta and gamma position to the Y attached to the phosphorus;

or together V and W are connected via an additional 3 carbon atoms to form an optionally substituted cyclic group containing 6 carbon atoms and substituted with one substituent selected from the group consisting of hydroxy, acyloxy, alkoxycarbonyloxy, alkylthiocarbonyloxy, and aryloxycarbonyloxy, attached to one of said carbon atoms that is three atoms from a Y attached to the phosphorus;

or together Z and W are connected via an additional 3-5 atoms to form a cyclic group, wherein 0-1 atoms are heteroatoms and the remaining atoms are carbon, and V must be aryl, substituted aryl, heteroaryl, or substituted heteroaryl;

or together W and W′ are connected via an additional 2-5 atoms to form a cyclic group, wherein 0-2 atoms are heteroatoms and the remaining atoms are carbon, and V must be aryl, substituted aryl, heteroaryl, or substituted heteroaryl;

Z is selected from the group consisting of —CHR^{z}OH, —CHR^{z}OC(O)R^{y}, —CHR^{z}OC(S)R^{y}, —CHR^{z}OC(S)OR^{y}, —CHR^{z}OC(O)SR^{y}, —CHR^{z}OCO₂R^{y}, —OR^{z}, —SR^{z}, —CHR^{z}N₃, —CH₂-aryl, —CH(aryl)OH, —CH(CH═CR^{z}₂)OH, —CH(C≡CR^{z})OH, —R^{z}, —NR^{z}₂, —OCOR^{y}, —OCO₂R^{y}, —SCOR^{y}, —SCO₂R^{y}, —NHCOR^{z}, —NHCO₂R^{y}, —CH₂NH-aryl, —(CH₂)q-OR^{z}, and —(CH₂)q-SR^{z};

q is an integer 2 or 3;

each R^{z} is selected from the group consisting of R^{y} and —H;

each R^{y} is selected from the group consisting of alkyl, aryl, heterocycloalkyl, and aralkyl;

each R^{x} is independently selected from the group consisting of —H, and alkyl, or together R^{x} and R^{x} form a cyclic alkyl group;

each R^{v} is selected from the group consisting of —H, lower alkyl, acyloxyalkyl, alkoxycarbonyloxyalkyl, and lower acyl;

and pharmaceutically acceptable salts and prodrugs thereof; and pharmaceutically acceptable salts of said prodrugs.

In some embodiments, the compound of Formula I has the following provisos:

a) when G is —O—, T is —CH₂—, R¹ and R² are each bromo, R³ is iso-propyl, R⁴ is hydrogen, and R⁵ is —OH, then X is not P(O)(OH)₂ or P(O)(OCH₂CH₃)₂;

b) V, Z, W, W′ are not all —H; and

c) when Z is —R^{z}, then at least one of V, W, and W′ is not —H, alkyl, aralkyl, or heterocycloalkyl;

d) when G is —O—, T is —(CH₂)₁₋₄—, R¹ and R² are independently halogen, alkyl, and cycloalkyl, R³ is alkyl, R⁴ is hydrogen, and R⁵ is —OH, then X is not —P(O)(OH)₂ or —P(O)(O-lower alkyl)₂; and

e) when G is —O—, R⁵ is —NHC(O)R^{e}, —NHS(═O)₁₋₂R^{e}, —NHC(S)NH(R^{b}), or —NHC(O)NH(R^{h}), T is —(CH₂)^{m}—, —CH═CH—, —O(CH₂)₁₋₂—, or —NH(CH₂)₁₋₂—, then X is not —P(O)(OH)₂ or —P(O)(OH)NH₂.

In some embodiments of Formula I:

G is selected from the group consisting of —O—, —S—, —S(═O)—, —S(═O)₂—, —CH₂—, —CF₂—, —CHF—, —C(O)—, —CH(OH)—, —NH—, and —N(C₁-C₄ alkyl)-;

T is selected from the group consisting of —(CR^{a}₂)ₖ—, CR^{b}═CR^{b}—(CR^{a}₂)ₙ—, —(CR^{a}₂)ₙ—CR^{b}═CR^{b}, —(CR^{a}₂)—CR^{b}═R^{b} (CR^{a}₂), —O(CR^{b}₂)(CR^{a}₂)ₙ—, —S(CR^{a}₂)(CR^{a}₂)ₙ, N(R^{b})(CR^{b}₂)(CR^{a}₂)ₙ, —N(R^{b})C(O)(CR^{a}₂)ₙ—, —(CR^{a}₂)ₙCH(NR^{b}R^{c})—, —C(O)(CR^{a}₂)ₘ—, —(CR^{a}₂)ₘC(O)—, —(CR^{a}₂)C(O)(CR^{a}₂)ₙ—, —(CR^{a}₂)ₙC(O)(CR^{a}₂) and —C(O)NH(CR^{b}₂)(CR^{a}₂)pr;

k is an integer from 0-4;

m is an integer from 0-3;

n is an integer from 0-2;

p is an integer from 0-1;

Each R^{a} is independently selected from the group consisting of hydrogen, optionally substituted —C₁-C₄ alkyl, halogen, —OH, optionally substituted —O—C₁-C₄ alkyl, —OCF₃, optionally substituted —S—C₁-C₄ alkyl, —NR^{b}R^{c}, optionally substituted —C₂-C₄ alkenyl, and optionally substituted —C₂-C₄ alkynyl; with the proviso that when one R^{a} is attached through O, S, or N, then the other R^{a} attached to the same C is a hydrogen, or attached via carbon atom. Each R^{b} is independently selected from the group consisting of hydrogen, optionally substituted —C₁-C₄ alkyl;

Each R^{c} is independently selected from the group consisting of hydrogen and optionally substituted —C₁-C₄ alkyl, optionally substituted —C(O)—C₁-C₄ alkyl, and —C(O)H;

R¹ and R² are each independently selected from the group consisting of halogen, optionally substituted —C₁-C₄ alkyl, optionally substituted —S—C₁-C₃ alkyl, optionally substituted —C₂-C₄ alkenyl, optionally substituted —C₂-C₄ alkynyl, —CF₃, —OCF₃, optionally substituted-O—C₁-C₃ alkyl, and cyano;

R³ and R⁴ are each independently selected from the group consisting of hydrogen, halogen, —CF₃, —OCF₃, cyano, optionally substituted —C₁-C₁₂ alkyl, optionally substituted —C₂-C₁₂ alkenyl, optionally substituted —C₂-C₁₂alkynyl, optionally substituted —(CR^{a}₂)ₘaryl, optionally substituted —(CR^{a}₂)ₘcycloalkyl, optionally substituted —(CR^{a}₂)ₘheterocycloalkyl, —OR^{d}, —SR^{d}, —S(═O)R^{e}, —S(═O)₂R^{e}, —S(═O)₂NR^{f}R^{g}, —C(O)NR^{e}R^{g}, —(O)OR^{h}, —C(O)R^{e}, —N(R)C(O)R^{e}, —N(R)C(O)NR^{e}R^{g}, —N(R^{b})S(═O)₂R^{e}, —N(R^{b})S(═O)₂NR^{f}R^{g}, and —NR^{f}R^{g};

Each R^{d} is selected from the group consisting of optionally substituted —C₁-C₁₂ alkyl, optionally substituted —C₂-C₁₂ alkenyl, optionally substituted —C₂-C₁₂ alkynyl, optionally substituted, —(CR^{b}₂)ₙaryl, optionally substituted, —(CR^{b}₂)ₙcycloalkyl, optionally substituted —(CR^{b}₂)ₙheterocycloalkyl, and —C(O)NR^{f}R^{g};

Each R^{e} is selected from the group consisting of optionally substituted —C₁-C₁₂ alkyl, optionally substituted —C₂-C₁₂ alkenyl, optionally substituted —C₂-C₁₂ alkynyl, optionally substituted —(CR^{a}₂)ₙaryl, optionally substituted, —(CR^{e}₂)ₙcycloalkyl, and optionally substituted —(CR^{a}₂)ₙheterocycloalkyl;

R^{f} and R^{g} are each independently selected from the group consisting of hydrogen, optionally substituted —C₁-C₁₂ alkyl, optionally substituted —C₂-C₁₂ alkenyl, optionally substituted —C₂-C₁₂ alkynyl, optionally substituted —(CR^{b}₂)ₙ aryl, optionally substituted —(CR^{b}₂)ₙ cycloalkyl, and optionally substituted —CR^{b}₂)ₙ heterocycloalkyl, or R^{f} and R^{g} may together form an optionally substituted heterocyclic ring, which may contain a second heterogroup selected from the group of O, NR^{c}, and S, wherein said optionally substituted heterocyclic ring may be substituted with 0-4 substituents selected from the group consisting of optionally substituted —C₁-C₄ alkyl, —OR^{b}, oxo, cyano, —CF₃, optionally substituted phenyl, and —C(O)OR^{h};

Each R^{h} is selected from the group consisting of optionally substituted —C₁-C₁₂ alkyl, optionally substituted —C₂-C₁₂ alkenyl, optionally substituted —C₂-C₁₂alkynyl, optionally substituted —(CR^{b}₂)ₙ aryl, optionally substituted —(CR^{b}₂)ₙ cycloalkyl, and optionally substituted —(CR^{b}₂)ₙ heterocycloalkyl;

R⁵ is selected from the group consisting of —OH, optionally substituted —OC₁-C₆ alkyl, —OC(O)R^{e}, —OC(O)OR^{h}, —F, —NHC(O)R^{e}, —NHS(═O)R^{e}, —NHS(═O)₂R^{e}, —NHC(═S)NH(R^{h}), and —NHC(O)NH(R^{h});

X is P(O)YR¹¹Y′R¹¹;

Y and Y′ are each independently selected from the group consisting of —O—, and —NR^{v}—; when Y and Y′ are —O—, R¹¹ attached to —O— is independently selected from the group consisting of —H, alkyl, optionally substituted aryl, optionally substituted heterocycloalkyl, optionally substituted CH₂-heterocycloakyl wherein the cyclic moiety contains a carbonate or thiocarbonate, optionally substituted -alkylaryl, —C(R^{z})₂OC(O)NR^{z}₂, —NR^{z}—C(O)—R^{y}, —C(R^{z})₂—OC(O)R^{y}, —C(R^{z})₂O—C(O)OR^{y}, —C(R^{z})₂OC(O)SR^{y}, -alkyl-S—C(O)R^{y}, -alkyl-S—S-alkylhydroxy, nd-alkyl-S—S—S-alkylhydroxy;

when Y and Y′ are —NR^{v}—, then R¹¹ attached to —NR^{v}— is independently selected from the group consisting of —H, —[C(R^{z})₂]_{q}—COOR^{y}, —C(R^{x})₂COOR^{y}, —[C(R^{z})₂]_{q}—C(O)SR^{y}, and -cycloalkylene-COOR^{y};

when Y is —O— and Y′ is NR^{v}, then R¹¹ attached to —O— is independently selected from the group consisting of —H, alkyl, optionally substituted aryl, optionally substituted heterocycloalkyl, optionally substituted CH₂-heterocycloakyl wherein the cyclic moiety contains a carbonate or thiocarbonate, optionally substituted -alkylaryl, —C(R^{z})₂OC(O)NR^{z}₂, —NR^{z}—C(O)—R^{y}, —C(R^{z})₂—OC(O)R^{y}, —C(R^{z})₂—O—C(O)OR^{y}, —C(R^{z})₂OC(O)SR^{y}, -alkyl-S—C(O)R^{y}, -alkyl-S—S-alkylhydroxy, and -alkyl-S—S—S-alkylhydroxy; and R¹ attached to —NR^{v}— is independently selected from the group consisting of —H, —[C(R^{z})₂]_{q}—COOR^{y}, —C(R^{x})₂COOR^{y}, —[C(R^{z})₂]_{q}—C(O)SR^{y}, and -cycloalkylene-COOR^{y};

or when Y and Y′ are independently selected from —O— and —NR^{v}—, then together R¹¹ and R¹¹ are -alkyl-S—S-alkyl- to form a cyclic group, or together R¹¹ and R¹¹ are the group:

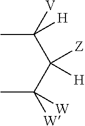

wherein:

V, W, and W′ are independently selected from the group consisting of hydrogen, optionally substituted alkyl, optionally substituted aralkyl, heterocycloalkyl, aryl, substituted aryl, heteroaryl, substituted heteroaryl, optionally substituted 1-alkenyl, and optionally substituted 1-alkynyl;

or together V and Z are connected via an additional 3-5 atoms to form a cyclic group containing 5-7 atoms, wherein 0-1 atoms are heteroatoms and the remaining atoms are carbon, substituted with hydroxy, acyloxy, alkylthiocarbonyloxy, alkoxycarbonyloxy, or aryloxycarbonyloxy attached to a carbon atom that is three atoms from both Y groups attached to the phosphorus; or

or together V and Z are connected via an additional 3-5 atoms to form a cyclic group, wherein 0-1 atoms are heteroatoms and the remaining atoms are carbon, that is fused to an aryl group at the beta and gamma position to the Y attached to the phosphorus;

or together V and W are connected via an additional 3 carbon atoms to form an optionally substituted cyclic group containing 6 carbon atoms and substituted with one substituent selected from the group consisting of hydroxy, acyloxy, alkoxycarbonyloxy, alkylthiocarbonyloxy, and aryloxycarbonyloxy, attached to one of said carbon atoms that is three atoms from a Y attached to the phosphorus;

or together Z and W are connected via an additional 3-5 atoms to form a cyclic group, wherein 0-1 atoms are heteroatoms and the remaining atoms are carbon, and V must be aryl, substituted aryl, heteroaryl, or substituted heteroaryl;

or together W and W′ are connected via an additional 2-5 atoms to form a cyclic group, wherein 0-2 atoms are heteroatoms and the remaining atoms are carbon, and V must be aryl, substituted aryl, heteroaryl, or substituted heteroaryl;

Z is selected from the group consisting of —CHR^{z}OH, —CHR^{z}OC(O)R^{y}, —CHR^{z}OC(S)R^{y}, —CHR^{z}OC(S)OR^{y}, —CHR^{z}OC(O)SR^{y}, —CR^{z}OCO₂R^{y}, —OR^{z}, —SR^{z}, —CHR^{z}N₃, —CH₂aryl, —CH(aryl)OH, —CH(CH═CR^{z}₂)OH, —CH(C≡CR^{z})OH, —R^{z}, —NR^{z}₂, —OCOR^{y}, —OCO₂R^{y}, —SCOR^{y}, —SCO₂R^{y}, —NHCOR^{z}, —NHCO₂R^{y}, —CH₂NHaryl, —(CH₂)_{q}—OR^{z}, and —(CH₂)_{q}—SR^{z};

q is an integer 2 or 3;

Each R^{z} is selected from the group consisting of R^{y} and —H;

Each R^{y} is selected from the group consisting of alkyl, aryl, heterocycloalkyl, and aralkyl;

Each R^{x} is independently selected from the group consisting of —H, and alkyl, or together R^{x} and R^{x} form a cyclic alkyl group;

Each R^{y} is selected from the group consisting of —H, lower alkyl, acyloxyalkyl, alkoxycarbonyloxyalkyl, and lower acyl;

with the provisos that:

a) when G is —O—, T is —CH₂—, R¹ and R² are each bromo, R³ is iso-propyl, R⁴ is hydrogen, and R⁵ is —H, then X is not P(O)(OH)₂ or P(O)(OCH₂CH₃)₂;

b) V, Z, W, W′ are not all —H; and

c) when Z is —R^{z}, then at least one of V, W, and W′ is not —H, alkyl, aralkyl, or heterocycloalkyl.

In other embodiments of Formula I:

G is selected from the group consisting of —O—, —S—, —S(═O)—, S(—O)₂—, —CH₂—, —CF₂—, —CHF—, —C(O)—, —CH(OH)—, —NH—, and —N(C₁-C₄ alkyl)-;

T is selected from the group consisting of —(CR^{a}₂)ₖ, —CR^{b}═CR^{b}—(CR^{a}₂)ₙ—, —(CR^{a}₂)—CR^{b}═CR^{b}—, —(CR^{a}₂)—CR^{b}═CR^{b}—(CR^{a}₂)—, —(CR, —(CR^{b}₂)(CR^{a}₂)ₙ—, —S(CR^{b}₂)(CR^{a}₂)ₙ—, N(R^{C})(CR^{b}₂)(CR^{a}₂)ₙ—, N(R)C(O)(CR^{a}₂)—, —(CR^{a}₂)ₙCH(NR^{b}R^{c})—, —C(O)(CR^{a}₂)ₘ, (CR^{a}₂)ₘC(O), —(CR^{a}₂)C(O)(CR^{a}₂)ₘ—(CR^{a}₂)ₙC(O)(CR^{a}₂)—, and C(O)NH(CR^{b}₂)(CR^{a}₂)ₚ;

k is an integer from 0-4;

m is an integer from 0-3;

n is an integer from 0-2;

p is an integer from 0-1;

Each R^{a} is independently selected from the group consisting of hydrogen, optionally substituted —C₁-C₄ alkyl, halogen, —OH, optionally substituted —O—C₁-C₄ alkyl, —OCF₃, optionally substituted —S—C₁-C₄ alkyl, —NR^{b}R^{c}, optionally substituted —C₂-C₄ alkenyl, and optionally substituted —C₂-C₄ alkynyl; with the proviso that when one R^{a} is attached to C through an O, S, or N atom, then the other R^{a} attached to the same C is a hydrogen, or attached via a carbon atom;

Each R^{b} is independently selected from the group consisting of hydrogen, optionally substituted —C₁-C₄ alkyl;

Each R^{c} is independently selected from the group consisting of hydrogen and optionally substituted —C₁-C₄ alkyl, optionally substituted —C(O)—C₁-C₄ alkyl, and —C(O)H;

R¹ and R² are each independently selected from the group consisting of halogen, optionally substituted —C₁-C₄alkyl, optionally substituted —S—C₁-C₃ alkyl, optionally substituted —C₂-C₄ alkenyl, optionally substituted —C₂-C₄ alkynyl, —CF₃, —OCF₃, optionally substituted-O—C₁-C₃ alkyl, and cyano;

R³ and R⁴ are each independently selected from the group consisting of hydrogen, halogen, —CF₃, —OCF₃, cyano, optionally substituted —C₁-C₁₂ alkyl, optionally substituted —C₂-C₁₂ alkenyl, optionally substituted —C₂-C₁₂ alkynyl, optionally substituted —(CR^{a}₂)ₘ aryl, optionally substituted —(CR^{a}₂)ₘ cycloalkyl, optionally substituted —(CR^{a}₂)ₘ heterocycloalkyl, —OR^{d}, —SR^{d}, —S(═O)R^{e}, —S(═O)₂R^{e}, —S(═O)₂NR^{f}R^{g}, —C(O)NR^{f}R^{g}, —C(O)OR^{h}, —C(O)R^{e}, N(R)C(O)R^{e}, —N(R)C(O)NR^{f}R^{g}, —N(R)S(═O)₂R^{e}, —N(R^{b})S(═O)₂NR^{f}R^{g}, and —NR^{f}R^{g};

Each R^{d} is selected from the group consisting of optionally substituted —C₁-C₁₂ alkyl, optionally substituted —C₂-C₁₂ alkenyl, optionally substituted —C₂-C₁₂ alkynyl, optionally substituted —(CR^{b}₂)ₙ aryl, optionally substituted —(CR^{b}₂)ₙ cycloalkyl, optionally substituted —(CR^{b}₂)ₙ heterocycloalkyl, and —C(O)NR^{f}R^{g};

Each R^{e} is selected from the group consisting of optionally substituted —C₁-C₁₂ alkyl, optionally substituted —C₂-C₁₂ alkenyl, optionally substituted —C₂-C₁₂ alkynyl, optionally substituted —(CR^{a}₂)ₙ aryl, optionally substituted —(CR^{a}₂)ₙ cycloalkyl, and optionally substituted —(CR^{a}₂)heterocycloalkyl;

R^{f} and R^{g} are each independently selected from the group consisting of hydrogen, optionally substituted C₁-C₁₂ alkyl, optionally substituted —C₂-C₁₂ alkenyl, optionally substituted —C₂-C₁₂alkynyl, optionally substituted —(CR^{b}₂)ₙ aryl, optionally substituted —(CR^{b}₂)ₙ cycloalkyl, and optionally substituted —(CR^{b}₂)ₙ heterocycloalkyl, or R^{f} and R^{g} may together form an optionally substituted heterocyclic ring, which may contain a second heterogroup selected from the group consisting of O, NR^{c}, and S, wherein said optionally substituted heterocyclic ring may be substituted with 0-4 substituents selected from the group consisting of optionally substituted —C₁-C₄ alkyl, —OR^{b}, OXO, cyano, —CF₃, optionally substituted phenyl, and —C(O)OR^{h};

Each R^{h} is selected from the group consisting of optionally substituted —C₁-C₁₂ alkyl, optionally substituted —C₂-C₁₂ alkenyl, optionally substituted —C₂-C₁₂ alkynyl, optionally substituted —(CR^{b}₂)ₙ aryl, optionally substituted —(CR^{b}₂)ₙ cycloalkyl, and optionally substituted —(CR^{b}₂)ₙ heterocycloalkyl;

R⁵ is selected from the group consisting of —OH, optionally substituted —OC₁-C₆ alkyl, —OC(O)R^{e}, —OC(O)OR^{h}, —F, —NHC(O)R^{e}, —NHS(═O)R^{e}, —NHS(═O)₂R^{e}, —NHC(═S)NH(R^{h}), and —NHC(O)NH(R^{h});

X is P(O)YR¹¹Y′R¹¹;

Y and Y′ are each independently selected from the group consisting of —O—, and —NR^{v}—; when Y and Y′ are —O—, R¹¹ attached to —O— is independently selected from the group consisting of —H, alkyl, optionally substituted aryl, optionally substituted heterocycloalkyl, optionally substituted CH₂-heterocycloakyl wherein the cyclic moiety contains a carbonate or thiocarbonate, optionally substituted -alkylaryl, —C(R^{z})₂OC(O)NR^{z}₂, —NR^{z}—C(O)—R^{y}, —C(R^{z})₂—OC(O)R^{y}, —C(R^{z})₂—O—C(O)OR^{y}, —C(R^{z})₂OC(O)SR^{y}, -alkyl-S—C(O)R^{y}, -alkyl-S—S-alkylhydroxy, and -alkyl-S—S—S-alkylhydroxy;

when Y and Y′ are —NR^{v}—, then R¹¹ attached to NR^{v}— is independently selected from the group consisting of —H, —[C(R^{z})₂]_{q}—COOR^{y}, —C(R^{x})₂COOR^{y}, —[C(R^{z})₂]_{q}—C(O)SR^{y}, and -cycloalkylene-COOR^{y};

when Y is —O— and Y′ is NR^{v}, then R¹¹ attached to —O— is independently selected from the group consisting of —H, alkyl, optionally substituted aryl, optionally substituted heterocycloalkyl, optionally substituted CH₂-heterocycloakyl wherein the cyclic moiety contains a carbonate or thiocarbonate, optionally substituted -alkylaryl, —C(R^{z})₂OC(O)NR^{z}₂, —NR^{z}—C(O)—R^{y}, —C(R^{z})₂—OC(O)R^{y}, —C(R^{z})₂—O—C(O)OR^{y}, —C(R^{z})₂OC(O)SR^{y}, -alkyl-S—C(O)R^{y}, -alkyl-S—S-alkylhydroxy, and -alkyl-S—S—S-alkylhydroxy; and R¹ attached to —NR^{v}— is independently selected from the group consisting of —H, —[C(R^{z})₂]_{q}—COOR^{y}, —C(R^{x})₂COOR^{y}, —[C(R^{z})₂]_{q}—C(O)SR^{y}, and -cycloalkylene-COOR^{y};

or when Y and Y′ are independently selected from —O— and —NR^{v}—, then together R¹¹ and R¹¹ are -alkyl-S—S-alkyl- to form a cyclic group, or together R¹¹ and R¹¹ are the group:

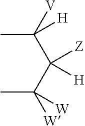

wherein:

V, W, and W′ are independently selected from the group consisting of hydrogen, optionally substituted alkyl, optionally substituted aralkyl, heterocycloalkyl, aryl, substituted aryl, heteroaryl, substituted heteroaryl, optionally substituted 1-alkenyl, and optionally substituted 1-alkynyl;

or together V and Z are connected via an additional 3-5 atoms to form a cyclic group containing 5-7 atoms, wherein 0-1 atoms are heteroatoms and the remaining atoms are carbon, substituted with hydroxy, acyloxy, alkylthiocarbonyloxy, alkoxycarbonyloxy, or aryloxycarbonyloxy attached to a carbon atom that is three atoms from both Y groups attached to the phosphorus; or

or together V and Z are connected via an additional 3-5 atoms to form a cyclic group, wherein 0-1 atoms are heteroatoms and the remaining atoms are carbon, that is fused to an aryl group at the beta and gamma position to the Y attached to the phosphorus;

or together V and W are connected via an additional 3 carbon atoms to form an optionally substituted cyclic group containing 6 carbon atoms and substituted with one substituent selected from the group consisting of hydroxy, acyloxy, alkoxycarbonyloxy, alkylthiocarbonyloxy, and aryloxycarbonyloxy, attached to one of said carbon atoms that is three atoms from a Y attached to the phosphorus;

or together Z and W are connected via an additional 3-5 atoms to form a cyclic group, wherein 0-1 atoms are heteroatoms and the remaining atoms are carbon, and V must be aryl, substituted aryl, heteroaryl, or substituted heteroaryl;

or together W and W′ are connected via an additional 2-5 atoms to form a cyclic group, wherein 0-2 atoms are heteroatoms and the remaining atoms are carbon, and V must be aryl, substituted aryl, heteroaryl, or substituted heteroaryl;

Z is selected from the group consisting of —CHR^{z}OH, —CHR^{z}OC(O)R^{y}, —CHR^{z}OC(S)R^{y}, —CHR^{z}OC(S)OR^{y}, —CHR^{z}OC(O)SR^{y}, —CHR^{z}OCO₂R^{y}, —OR^{z}, —SR^{z}, —CHR^{z}N₃, —CH₂aryl, —CH(aryl)OH, —CH(CH═CR^{z}₂)OH, —CH(C≡CR^{z})OH, —R^{z}, —NR^{z}₂, —OCOR^{y}, —OCO₂R^{y}, —SCOR^{y}, —SCO₂R^{y}, —NHCOR^{z}, —NHCO₂R^{y}, —CH₂NHaryl, —(CH₂)_{q}—OR^{z}, and —(CH₂)_{q}—SR^{z};

q is an integer 2 or 3;

Each R^{z} is selected from the group consisting of R^{y} and —H;

Each R^{y} is selected from the group consisting of alkyl, aryl, heterocycloalkyl, and aralkyl;

Each R^{x} is independently selected from the group consisting of —H, and alkyl, or together R^{x} and R^{x} form a cyclic alkyl group;

Each R^{v} is selected from the group consisting of —H, lower alkyl, acyloxyalkyl, alkoxycarbonyloxyalkyl, and lower acyl;

with the proviso that when G is —O—, T is —CH₂—, R¹ and R² are each bromo, R³ is iso-propyl, R⁴ is hydrogen, and R⁵ is —OH, then X is not P(O)(OH)₂ or P(O)(OCH₂CH₃)₂;

and pharmaceutically acceptable salts and prodrugs thereof and pharmaceutically acceptable salts of said prodrugs.

In various other embodiments of Formula I:

G is selected from the group consisting of —O—, —S—, —S(═O)—, —S(═O)₂—, —CH₂—, —CF₂—, —CHF—, —C(O)—, —CH(OH)—, —NH—, and —N(C₁-C₄ alkyl)-;

T is selected from the group consisting of —(CR^{a}₂)ₖ—, —CR^{b}═CR^{b}—(CR^{a}₂)ₙ—, —(CR^{a}₂)ₙ—CR^{b}═CR^{b}, —(CR^{a}₂)—CR^{b} CR^{b}—(CR^{a}₂)—, —O(CR^{b}₂)(CR^{a}₂)ₙ—, —S(CR^{b}₂)(CR^{a}₂)ₙ—, —N(R^{c})—(CR^{b}₂)(CR^{a}₂)—, —N(R^{b})C(O)(CR^{a}₂)ₙ—, —(CR^{a}₂)ₙCH(NR^{b}R^{c})—, C(O)(CR^{a}₂)ₘ—, —(CR^{a}₂)ₘC(O)—, —(CR^{a}₂)C(O)(CR^{a}₂)ₙ—, —(CR^{a}₂)ₙC(O)(CR^{a}₂)—, and C(O)NH(CR^{b}₂)(CR^{a}₂)ₚ;

k is an integer from 0-4;

m is an integer from 0-3;

n is an integer from 0-2;

p is an integer from 0-1;

Each R^{a} is independently selected from the group consisting of hydrogen, optionally substituted —C₁-C₄ alkyl, halogen, —OH, optionally substituted —O—C₁-C₄ alkyl, —OCF₃, optionally substituted —S—C₁-C₄ alkyl, —NR^{b}R^{c}, optionally substituted —C₂-C₄ alkenyl, and optionally substituted —C₂-C₄ alkynyl; with the proviso that when one R^{a} is attached to C through an O, S, or N atom, then the other R^{a} attached to the same C is a hydrogen, or attached via a carbon atom;

Each R^{b} is independently selected from the group consisting of hydrogen and optionally substituted —C₁-C₄ alkyl;

Each R^{c} is independently selected from the group consisting of hydrogen, optionally substituted —C₁-C₄ alkyl, optionally substituted —C(O)—C₁-C₄ alkyl, and —C(O)H;

R¹ and R² are each independently selected from the group consisting of halogen, optionally substituted —C₁-C₄alkyl, optionally substituted —S—C₁-C₃ alkyl, optionally substituted —C₂-C₄ alkenyl, optionally substituted —C₂-C₄ alkynyl, —CF₃, —OCF₃, optionally substituted-O—C₁-C₃ alkyl, and cyano;

R³ and R⁴ are each independently selected from the group consisting of hydrogen, halogen, —CF₃, —OCF₃, cyano, optionally substituted —C₁-C₁₂ alkyl, optionally substituted —C₂-C₁₂ alkenyl, optionally substituted —C₂-C₁₂ alkynyl, optionally substituted —(CR^{a}₂)ₘaryl, optionally substituted —(CR^{a}₂)ₘcycloalkyl, optionally substituted (CR^{a}₂)ₘheterocycloalkyl, —OR^{d}, —SR^{d}, —S(═O)R^{e}, —S(═O)₂R^{e}, —S(═O)₂R^{f}R^{g}, —C(O)NR^{f}R^{g}, —C(O)OR^{h}, —C(O)R^{e}, N(R)C(O)R^{e}, —N(R)C(O)NR^{f}R^{g}, N(R^{b})S(═O)₂R^{e}, —N(R^{b})S(═O)₂NR^{f}R^{g}, and —NR^{f}R^{g};

Each R^{d} is selected from the group consisting of optionally substituted —C₁-C₁₂ alkyl, optionally substituted —C₂-C₁₂ alkenyl, optionally substituted —C₂-C₁₂ alkynyl, optionally substituted —(CR^{b}₂)ₙaryl, optionally substituted —(CR^{b}₂)ₙcycloalkyl, optionally substituted —(CR^{b}₂)ₙheterocycloalkyl, and —C(O)NR^{f}R^{g};

Each R^{e} is optionally substituted —C₁-C₁₂ alkyl, optionally substituted —C₂-C₁₂ alkenyl, optionally substituted —C₂-C₁₂alkynyl, optionally substituted —(CR^{b}₂)ₙaryl, optionally substituted —(CR^{b}₂)ₙcycloalkyl, and optionally substituted —(CR^{b}₂)ₙheterocycloalkyl;

R^{f} and R^{g} are each independently selected from the group consisting of hydrogen, optionally substituted —C₁-C₁₂ alkyl, optionally substituted —C₂-C₁₂ alkenyl, optionally substituted —C₂-C₁₂ alkynyl, optionally substituted —(CR^{b}₂)ₙaryl, optionally substituted —(CR^{b}₂)ₙcycloalkyl, and optionally substituted —(CR^{b}₂)ₙheterocycloalkyl, or R^{f} and R^{g} may together form an optionally substituted heterocyclic ring, which may contain a second heterogroup selected from the group of O, NR^{c}, and S, wherein said optionally substituted heterocyclic ring may be substituted with 0-4 substituents selected from the group consisting of optionally substituted —C₁-C₄ alkyl, —OR^{b}, oxo, cyano, —CF₃, optionally substituted phenyl, and —C(O)OR^{h};

Each R^{h} is selected from the group consisting of optionally substituted —C₁-C₁₂ alkyl, optionally substituted —C₂-C₁₂ alkenyl, optionally substituted —C₂-C₁₂ alkynyl, optionally substituted —(CR^{b}₂)ₙaryl, optionally substituted —(CR^{b}₂)ₙcycloalkyl, and optionally substituted —(CR^{b}₂)ₙheterocycloalkyl;

R⁵ is selected from the group consisting of —OH, optionally substituted —OC₁-C₆ alkyl, —OC(O)R^{e}, —OC(O)OR^{h}, —F, —NHC(O)R^{e}, —NHS(═O)R^{e}, —NHS(═O)₂R^{e}, —NHC(═S)NH(R^{h}), and —NHC(O)NH(R);

X is P(O)YR¹¹Y′R¹¹;

Y and Y′ are each independently selected from the group consisting of —O—, and —NR^{v}—; when Y and Y′ are —O—, R¹¹ attached to —O— is independently selected from the group consisting of —H, alkyl, optionally substituted aryl, optionally substituted heterocycloalkyl, optionally substituted CH₂-heterocycloakyl wherein the cyclic moiety contains a carbonate or thiocarbonate, optionally substituted -alkylaryl, —C(R^{z})₂OC(O)NR^{z}₂, —NR^{z}—C(O)—R^{y}, —C(R^{z})₂—OC(O)R^{y}, —C(R^{z})₂—O—C(O)OR^{y}, —C(R^{z})₂OC(O)SR^{y}, -alkyl-S—C(O)R^{y}, -alkyl-S—S-alkylhydroxy, and -alkyl-S—S—S-alkylhydroxy;

when Y and Y′ are —NR^{v}—, then R¹¹ attached to —NR^{v}— is independently selected from the group consisting of —H, —[C(R^{z})₂]_{q}—COOR^{y}, —C(R^{x})₂COOR^{y}, —[C(R^{z})₂]_{q}—C(O)SR^{y}, and -cycloalkylene-COOR^{y};

when Y is —O— and Y′ is NR^{v}, then R¹¹ attached to —O— is independently selected from the group consisting of —H, alkyl, optionally substituted aryl, optionally substituted heterocycloalkyl, optionally substituted CH₂-heterocycloakyl wherein the cyclic moiety contains a carbonate or thiocarbonate, optionally substituted -alkylaryl, —C(R^{z})₂OC(O)NR^{e}₂, —NR^{z}—C(O)—R^{y}, —C(R^{z})₂—OC(O)R^{y}, —C(R^{z})₂—O—C(O)OR^{y}, —C(R^{z})₂OC(O)SR^{y}, -alkyl-S—C(O)R^{y}, -alkyl-S—S-alkylhydroxy, and -alkyl-S—S—S-alkylhydroxy; and R¹ attached to —NR^{v}— is independently selected from the group consisting of —H, —[C(R^{z})₂]_{q}—COOR^{y}, —C(R^{x})₂COOR^{y}, —[C(R^{z})₂]_{q}—C(O)SR^{y}, and -cycloalkylene-COOR^{y};

or when Y and Y′ are independently selected from —O— and —NR^{v}—, then together R¹¹ and R¹¹ are -alkyl-S—S-alkyl- to form a cyclic group, or together R¹¹ and R¹¹ are the group:

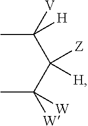

wherein:

V, W, and W′ are independently selected from the group consisting of hydrogen, optionally substituted alkyl, optionally substituted aralkyl, heterocycloalkyl, aryl, substituted aryl, heteroaryl, substituted heteroaryl, optionally substituted 1-alkenyl, and optionally substituted 1-alkynyl;

or together V and Z are connected via an additional 3-5 atoms to form a cyclic group containing 5-7 atoms, wherein 0-1 atoms are heteroatoms and the remaining atoms are carbon, substituted with hydroxy, acyloxy, alkylthiocarbonyloxy, alkoxycarbonyloxy, or aryloxycarbonyloxy attached to a carbon atom that is three atoms from both Y groups attached to the phosphorus; or

or together V and Z are connected via an additional 3-5 atoms to form a cyclic group, wherein 0-1 atoms are heteroatoms and the remaining atoms are carbon, that is fused to an aryl group at the beta and gamma position to the Y attached to the phosphorus;

or together V and W are connected via an additional 3 carbon atoms to form an optionally substituted cyclic group containing 6 carbon atoms and substituted with one substituent selected from the group consisting of hydroxy, acyloxy, alkoxycarbonyloxy, alkylthiocarbonyloxy, and aryloxycarbonyloxy, attached to one of said carbon atoms that is three atoms from a Y attached to the phosphorus;

or together Z and W are connected via an additional 3-5 atoms to form a cyclic group, wherein 0-1 atoms are heteroatoms and the remaining atoms are carbon, and V must be aryl, substituted aryl, heteroaryl, or substituted heteroaryl;

or together W and W′ are connected via an additional 2-5 atoms to form a cyclic group, wherein 0-2 atoms are heteroatoms and the remaining atoms are carbon, and V must be aryl, substituted aryl, heteroaryl, or substituted heteroaryl;

Z is selected from the group consisting of —CHR^{z}OH, —CHR^{z}OC(O)R^{y}, —CHR^{z}OC(S)R^{y}, —CHR^{z}OC(S)OR^{y}, —CHR^{z}OC(O)SR^{y}, —CHR^{z}OCO₂R^{y}, —OR^{z}, —SR^{z}, —CHR^{z}N₃, —CH₂aryl, —CH(aryl)OH, —CH(CH═CR^{z}₂)OH, —CH(C≡CR^{z})OH, —R^{z}, —NR^{z}₂, —OCOR^{y}, —OCO₂R^{y}, —SCOR^{y}, —SCO₂R^{y}, —NHCOR^{z}, —NHCO₂R^{y}, —CH₂NHaryl, —(CH₂)_{q}—OR^{z}, and —(CH₂)_{q}—SR^{z};

q is an integer 2 or 3;

Each R^{z} is selected from the group consisting of R^{y} and —H;

Each R^{y} is selected from the group consisting of alkyl, aryl, heterocycloalkyl, and aralkyl;

Each R^{x} is independently selected from the group consisting of —H, and alkyl, or together R^{x} and R^{x} form a cyclic alkyl group;

Each R^{v} is selected from the group consisting of —H, lower alkyl, acyloxyalkyl, alkoxycarbonyloxyalkyl, and lower acyl;

with the provisos that:

a) when G is —O—, T is —(CH₂)₀₋₄—, R¹ and R² are independently halogen, alkyl of 1 to 3 carbons, and cycloalkyl of 3 to 5 carbons, R³ is alkyl of 1 to 4 carbons or cycloalkyl of 3 to 7 carbons, R⁴ is hydrogen, and R⁵ is —OH, then X is not —P(O)(OH)₂ or —P(O)(O-lower alkyl)₂;

b) when G is —O—, R⁵ is —NHC(O)R^{e}, —NHS(═O)₁₋₂R^{e}, —NHC(S)NH(R^{h}), or —NHC(O)NH(R^{h}), T is —(CH₂)ₘ—, —CH═CH—, —O(CH₂)₁₋₂—, or —NH(CH₂)₁₋₂—, then X is not —P(O)(OH)₂ or —P(O)(OH)NH₂;

and pharmaceutically acceptable salts and prodrugs thereof and pharmaceutically acceptable salts of said prodrugs.

In one aspect, G is —O—. In another aspect, G is —CH₂—. In yet another aspect, G is selected from the group consisting of —O— and —CH₂—. In another aspect, G is —S—. In a further aspect, G is —S(═O)—. In another aspect, G is —S(═O)₂—. In a further aspect, G is —CH₂—. In another aspect, G is —CF₂—. In a further aspect, G is —CHF—. In another aspect, G is —C(O)—. In another aspect, G is —CH(OH)—. In a further aspect, G is —NH—.

In another aspect, G is —N(C₁-C₄ alkyl)-. In yet another aspect, G is selected from the group consisting of —O—, —S— and —CH₂—.

In one aspect, T is T is —CH₂—. In another aspect, T is —(CH₂)₀₋₄—. In another aspect, T is selected from the group consisting of —(CH₂)ₘ—, —CH═CH—, —O(CH₂)₁₋₂—, and —NH(CH₂)₁₋₂—. In yet another aspect, T is selected from the group consisting of (CR^{a}₂)ₙ, O(CR^{b}₂)(CR^{a}₂)ₚ, N(CR^{b}₂)(CR^{a}₂)ₚ, S(CR^{b}₂)(CR^{a}₂)ₚ—NR^{b}(CO)—, and —CH₂CH(NR^{c}R^{b}). In another aspect, T is —CH₂CH(NH₂)—. In another aspect, T is —N(H)C(O)—. In a further aspect, T is —OCH₂—. In another aspect, T is —CH₂CH₂—. In yet another aspect, T is —CH₂CH(NH₂)—. In another aspect, T is —N(H)C(O)—.

In a further aspect, T is —(CR^{a}₂)ₖ—. In another aspect, T is —CR^{b}CR^{b}—(CR^{a}₂)ₙ—. In a further aspect, T is —(CR^{a}₂)ₙ—CR^{b}—CR^{b}—. In another aspect, T is —(CR^{a}₂)—CR^{b}═CR^{b}—(CR^{a}₂)—. In a further aspect, T is —O(CR^{b}₂)(CR^{a}₂)ₙ—. In another aspect, T is —S(CR^{b}₂)(CR^{a}₂)ₙ—. In a further aspect, T is —N(CR^{b}₂)(CR^{a}₂)ₙ—. In another aspect, T is —N(R^{b})C(O)(CR^{a}₂)ₙ—. In a further aspect, T is —(CR^{e}₂)ₙCH(NR^{b}R^{c})—. In another aspect, T is —C(O)(CR^{a}₂)ₙ—. In a further aspect, T is —(CR^{a}₂)ₘC(O)—. In another aspect, T is —(CR^{a}₂)C(O)(CR^{a}₂)ₙ—. In a further aspect, T is —(CR^{a}₂)ₙC(O)(CR^{a}₂)—. In yet another aspect, T is —C(O)NH(CR^{b}₂)(CR^{a}₂)ₚ.

In one aspect k is 0. In a further aspect, k is 1. In an additional aspect, k is 2. In a further aspect, k is 3. In yet another aspect, k is 4. In one aspect m is 0. In a further aspect, m is 1. In an additional aspect, m is 2. In a further aspect, m is 3. In one aspect n is 0. In a further aspect, n is 1. In an additional aspect, n is 2. In one aspect, p is 0. In another aspect, p is 1.

In one aspect, each R^{a} is hydrogen with the proviso that when one R^{a} is attached to C through an O, S, or N atom, then the other R^{a} attached to the same C is a hydrogen, or attached via a carbon atom. In another aspect, each R^{a} is optionally substituted —C₁-C₄ alkyl with the proviso that when one R^{a} is attached to C through an O, S, or N atom, then the other R^{a} attached to the same C is a hydrogen, or attached via a carbon atom. In a further aspect, each R^{a} is halogen with the proviso that when one R^{a} is attached to C through an O, S, or N atom, then the other R^{a} attached to the same C is a hydrogen, or attached via a carbon atom. In another aspect, each R^{a} is —OH with the proviso that when one R^{a} is attached to C through an O, S, or N atom, then the other R^{a} attached to the same C is a hydrogen, or attached via a carbon atom. In a further aspect, each R^{a} is optionally substituted —O—C₁-C₄ alkyl with the proviso that when one R^{a} is attached to C through an O, S, or N atom, then the other R^{a} attached to the same C is a hydrogen, or attached via a carbon atom. In another aspect, each R^{a} is —OCF₃ with the proviso that when one R^{a} is attached to C through an O, S, or N atom, then the other R^{a} attached to the same C is a hydrogen, or attached via a carbon atom. In a further aspect, each R^{a} is optionally substituted —S—C₁-C₄ alkyl with the proviso that when one R^{a} is attached to C through an O, S, or N atom, then the other R^{a} attached to the same C is a hydrogen, or attached via a carbon atom. In another aspect, each R^{a} is —NR^{b}R^{c} with the proviso that when one R^{a} is attached to C through an O, S, or N atom, then the other R^{a} attached to the same C is a hydrogen, or attached via a carbon atom. In a further aspect, each R^{a} is optionally substituted —C₂-C₄ alkenyl with the proviso that when one R^{a} is attached to C through an O, S, or N atom, then the other R^{a} attached to the same C is a hydrogen, or attached via a carbon atom. In another aspect, each R^{a} is optionally substituted —C₂-C₄ alkynyl with the proviso that when one R^{a} is attached to C through an O, S, or N atom, then the other R^{a} attached to the same C is a hydrogen, or attached via a carbon atom.

In one aspect, R^{b} is hydrogen. In an additional aspect, R^{b} is optionally substituted —C₁-C₄ alkyl.

In one aspect, R^{c} is hydrogen. In another aspect, R^{c} is optionally substituted —C₁-C₄ alkyl. In a further aspect, R^{c} is optionally substituted —C(O)—C₁-C₄ alkyl. In yet another aspect, R^{c} is —C(O)H.

In one aspect, R¹ and R² are each bromo. In another aspect, R¹ and R² are independently selected from the group consisting of hydrogen, halogen, alkyl of 1 to 3 carbons, and cycloalkyl of 3 to 5 carbons. In another aspect, R¹ and R² are independently halogen, alkyl of 1 to 3 carbons, and cycloalkyl of 3 to 5 carbons, In a further aspect, R¹ and R² are the same and are selected from the group consisting of halogen, —C₁-C₄ alkyl, —CF₃, and cyano. In an additional aspect, R¹ and R² are different and are selected from the group consisting of halogen, —C₁-C₄ alkyl, —CF₃, and cyano. In one aspect, R¹ and R² are each independently selected from the group consisting of halogen, —C₁-C₄ alkyl, —CF₃, and cyano. In another aspect, R¹ and R² are each independently selected from the group consisting of iodo, bromo, chloro, methyl, and cyano. In another aspect, R¹ and R² are each iodo. In one aspect, R¹ and R² are each methyl. In a further aspect, R¹ and R² are each chloro. In another aspect, R¹ and R² are each independently selected from the group consisting of iodo, bromo, chloro, and methyl.

In an additional aspect, R¹ and R² are each halogen. In another aspect, R¹ and R² are each optionally substituted —C₁-C₄ alkyl. In a further aspect, R¹ and R² are each optionally substituted —S—C₁-C₃ alkyl. In another aspect, R¹ and R² are each optionally substituted —C₂-C₄ alkenyl. In a further aspect, R¹ and R² are each optionally substituted —C₂-C₄ alkynyl. In another aspect, R¹ and R² are each —CF₃. In a further aspect, R¹ and R² are each —OCF₃. In another aspect, R¹ and R² are each optionally substituted-O—C₁-C₃ alkyl. In a further aspect, R¹ and R² are each cyano.

In yet another aspect, R³ and R⁴ are each hydrogen. In another aspect, R³ and R⁴ are each halogen. In a further aspect, R³ and R⁴ are each —CF₃. In another aspect, R³ and R⁴ are each —OCF₃. In a further aspect, R³ and R⁴ are each cyano. In another aspect, R³ and R⁴ are each optionally substituted —C₁-C₁₂ alkyl. In a further aspect, R³ and R⁴ are each optionally substituted —C₂-C₁₂ alkenyl. In another aspect, R³ and R⁴ are each optionally substituted —C₂-C₁₂ alkynyl. In a further aspect, R³ and R⁴ are each optionally substituted —(CR^{a}₂)ₘaryl. In another aspect, R³ and R⁴ are each optionally substituted —(CR^{a}₂)ₘcycloalkyl. In a further aspect, R³ and R⁴ are each optionally substituted —(CR^{a}₂)ₙheterocycloalkyl. In another aspect, R³ and R⁴ are each —OR^{d}. In another aspect, R³ and R⁴ are each —SR^{d}. In a further aspect, R³ and R⁴ are each —S(═O)R^{e}. In another aspect, R³ and R⁴ are each —S(═O)₂R^{e}. In a further aspect, R³ and R⁴ are each —S(═O)₂NR^{f}R^{g}. In another aspect, R³ and R⁴ are each —C(O)NR⁹. In a further aspect, R³ and R⁴ are each —C(O)OR^{h}. In another aspect, R³ and R⁴ are each —C(O)R^{e}. In a further aspect, R³ and R⁴ are each —N(R^{b})C(O)R^{e}. In another aspect, R³ and R⁴ are each —N(R)C(O)NR^{f}R^{g}. In a further aspect, R³ and R⁴ are each —N(R)S(═O)₂R^{e}. In another aspect, R³ and R⁴ are each —N(R^{b})S(═O)₂NR^{f}R^{g}. In a further aspect, R¹ and R⁴ are each —NR^{f}R^{g}.

In one aspect, R⁴ is selected from the group consisting of hydrogen, halogen, —C₁-C₄ alkyl, cyano and CF₃. In another aspect, R⁴ is not hydrogen. In a further aspect, R⁴ is selected from the group consisting of hydrogen and halogen. In another aspect, R⁴ is selected from the group consisting of hydrogen and iodo. In a further aspect, R⁴ is hydrogen.

In another aspect, each R^{d} is optionally substituted —C₁-C₁₂ alkyl. In a further aspect, each R^{d} is optionally substituted —C₂-C₁₂ alkenyl. In another aspect, each R^{d} is optionally substituted —C₂-C₁₂ alkynyl. In a further aspect, each R^{d} is optionally substituted —(CR^{b}₂)ₙ aryl. In another aspect, each R^{d} is optionally substituted —(CR^{b}₂)ₙ cycloalkyl. In a further aspect, each R^{d} is optionally substituted —(CR^{b}₂)ₙ heterocycloalkyl. In another aspect, each R^{d} is —C(O)NR^{f}R^{g}.

In an additional aspect, R^{e} is optionally substituted —C₁-C₁₂ alkyl. In another aspect, R^{e} is optionally substituted —C₂-C₁₂ alkenyl. In a further aspect, R^{a} is optionally substituted —C₂-C₁₂ alkynyl. In another aspect, R^{e} is optionally substituted —CR^{a}₂)ₙ aryl. In a further aspect, R^{e} is optionally substituted —(CR^{b}₂)ₙ cycloalkyl. In another aspect, R^{e} is optionally substituted —(CR^{a}₂)ₙ heterocycloalkyl.

In one aspect, R^{f} and R_{g} are each hydrogen. In an additional aspect, R^{f} and R_{g} are each optionally substituted —C₁-C₁₂ alkyl. In another aspect, R^{f} and R_{g} are each optionally substituted —C₂-C₁₂ alkenyl. In an additional aspect, R^{f} and R^{g} are each optionally substituted —C₂-C₁₂ alkynyl. In a further aspect, R^{f} and R^{g} are each optionally substituted —(CR^{b}₂)ₙ aryl. In an additional aspect, R^{f} and R^{g} are each optionally substituted —(CR^{b}₂)ₙcycloalkyl. In another aspect, R^{f} and R^{g} are each optionally substituted —(CR^{b}₂)ₙheterocycloalkyl.

In an additional aspect, R^{f} and R^{g} may together form an optionally substituted heterocyclic ring, which may contain a second heterogroup which is O. In another aspect, R^{f} and R^{g} may together form an optionally substituted heterocyclic ring, which may contain a second heterogroup which is NR^{c}. In another aspect, R^{f} and R^{g} may together form an optionally substituted heterocyclic ring, which may contain a second heterogroup which is S. In one aspect, R^{f} and R^{g} may together form an unsubstituted heterocyclic ring, which may contain a second heterogroup. In another aspect, the optionally substituted heterocyclic ring may be substituted with 1 substituent selected from the group consisting of optionally substituted —C₁-C₄ alkyl, —OR^{b}, oxo, cyano, —CF₃, optionally substituted phenyl, and —C(O)OR^{h}. In further aspect, the optionally substituted heterocyclic ring may be substituted with 2 substituents selected from the group consisting of optionally substituted —C₁-C₄ alkyl, —OR^{h}, oxo, cyano, —CF₃, optionally substituted phenyl, and —C(O)OR^{h}. In another aspect, the optionally substituted heterocyclic ring may be substituted with 3 substituents selected from the group consisting of optionally substituted —C₁-C₄ alkyl, —OR^{b}, oxo, cyano, —CF₃, optionally substituted phenyl, and —C(O)OR^{h}. In a further aspect, the optionally substituted heterocyclic ring may be substituted with 4 substituents selected from the group consisting of optionally substituted —C₁-C₄ alkyl, —OR^{b}, oxo, cyano, —CF₃, optionally substituted phenyl, and —C(O)OR^{h}.

In a further aspect, R^{h} is optionally substituted —C₁-C₁₂ alkyl. In another aspect, R^{h} is optionally substituted —C₂-C₁₂ alkenyl. In a further aspect, R^{h} is optionally substituted —C₂-C₁₂ alkynyl. In another aspect, R^{h} is optionally substituted —(CR^{b}₂)ₙaryl. In a further aspect, R^{h} is optionally substituted —(CR^{b}₂)ₙ cycloalkyl. In another aspect, R^{h} is optionally substituted —(CR^{b}₂)ₙ heterocycloalkyl.

In one aspect, R⁵ is —OH. In another aspect, R⁵ is selected from the group consisting of —OH, —OC(O)R^{e}, —OC(O)OR^{h}, —F, and —NHC(O)R^{e}. In a further aspect, R⁵ is selected from the group consisting of —OH and —OC(O)R^{e}. In an additional aspect, R⁵ is optionally substituted —OC₁-C₆ alkyl. In another aspect, R⁵ is —OC(O)R^{e}. In a further aspect, R⁵ is —OC(O)OR^{h}. In another aspect, R⁵ is —F. In another aspect, R⁵ is —NHC(O)R^{e}. In a further aspect, R⁵ is —NHS(═O)R^{e}. In another aspect, R⁵ is —NHS(═O)₂R^{e}. In a further aspect, R⁵ is —NHC(═S)NH(R^{h}). In another aspect, R⁵ is —NHC(O)NH(Oh).

In one aspect, R³ is selected from the group consisting of halogen, optionally substituted —C₁-C₆ alkyl, —CF₃, cyano, —C(O)NR^{f}R^{g}, optionally substituted (CR^{a}₂)ₙaryl, —SO₂NR^{f}R^{g}, and —SO₂R^{e}. In another aspect, R³ is iso-propyl. In a further aspect, R³ is alkyl of 1 to 4 carbons or cycloalkyl of 3 to 7 carbons. In yet another aspect, R³ is selected from the group consisting of halogen, optionally substituted —C₁-C₆ alkyl, optionally substituted —CH₂aryl, optionally substituted —CH(OH)aryl, —C(O)-amido, S(═O)₂-amido, wherein the amido group is selected from the group consisting of phenethylamino, piperidinyl, 4-methylpiperizinyl, morpholinyl, cyclohexylamino, anilinyl, and indolinyl, and —SO₂R^{a} wherein R^{e} is selected from the group consisting of phenyl, 4-chlorophenyl, 4-fluorophenyl, and 4-pyridyl. In another aspect, R³ is iodo. In yet another aspect, R³ is selected from the group consisting of iodo, bromo, optionally substituted —C₁-C₆ alkyl, optionally substituted —CH₂aryl, optionally substituted —CH(OH)aryl, —(O)-amido, —S(═O)₂-amido, wherein the amido group is selected from the group consisting of phenethylamino, piperidinyl, 4-methypiperizinyl, morpholinyl, cyclohexylamino, anilinyl, and indolinyl, and —SO₂R^{e} wherein R^{e} is selected from the group consisting of phenyl, 4-chlorophenyl, 4-fluorophenyl, and 4-pyridyl. In one aspect, R³ is —CH(OH)(4-fluorophenyl).

In one aspect, X is —P(O)YR¹Y′R¹¹.

In one aspect, X is selected from the group consisting of —PO₃H₂, —P(O)[—OCR^{z}₂OC(O)R^{y}]₂, —P(O)[—OCR^{z}₂OC(O)OR^{y}]₂, —P(O)[—N(H)CR^{z}₂C(O)OR^{y}]₂, —P(O)[—N(H)CR^{z}₂C(O)OR¹][—OR¹¹], and —P(O)[—OCH(V)CH₂CH₂O—], wherein V is selected from the group consisting of optionally substituted aryl, aryl, heteroaryl, and optionally substituted heteroaryl. In another aspect, is selected from the group consisting of —PO₃H₂, —P(O)[—OCR^{z}₂OC(O)R^{y}]₂, —P(O)[—OCR^{z}₂OC(O)OR^{y}]₂, —P(O)[—OCH₂CH₂SC(O)Me]₂, —P(O)[—N(H)CR^{z}₂C(O)OR^{y}]₂, —P(O)[—N(H)CR^{z}₂C(O)OR][—OR¹¹] and —P(O)[—OCH(V)CH₂CH₂O—], wherein V is selected from the group consisting of optionally substituted aryl, aryl, heteroaryl, and optionally substituted heteroaryl. In another aspect, X is selected from the group consisting of —PO₃H₂, —P(O)[—OCR^{z}₂OC(O)R^{y}]₂, —P(O)[—OCR^{z}₂OC(O)OR^{y}]₂, —P(O)[—Oalk- SC(O)R^{y}]₂, —P(O)[—N(H)CR^{z}₂C(O)OR^{y}]₂, —P(O)[—N(H)CR^{z}₂C(O)OR][—OR¹¹] and —P(O)[—OCH(V)CH₂CH₂O—], wherein V is selected from the group consisting of optionally substituted aryl, aryl, heteroaryl, and optionally substituted heteroaryl. In one aspect, X is selected from the group consisting of —PO₃H₂, —P(O)[—OCH₂OC(O)-t-butyl]₂, —P(O)[—OCH₂OC(O)O-i-propyl]₂, —P(O)[—N(H)CH(CH₃)C(O)OCH₂CH₃]₂, —P(O)[—N(H)C(CH₃)₂C(O)OCH₂CH₃]₂, —P(O)[—N(H)CH(CH₃)C(O)OCH₂CH₃][3,4-methylenedioxyphenyl], —P(O)[—N(H)C(CH₃)₂C(O)OCH₂CH₃][3,4-methylenedioxyphenyl], —P(O)[—O—CH₂CH₂S—C(O)CH₃]₂, and —P(O)[—OCH(3-chlorophenyl)CH₂CH₂O—]. In a further aspect, X is selected from the group consisting of —PO₃H₂, —P(O)[—OCH₂OC(O)-t-butyl]₂, —P(O)[—OCH₂OC(O)O-i-propyl]₂, —P(O)[—N(CH(CH₃)C(O)OCH₂CH₃]₂, —P(O)[—N(H)C(CH₃)₂C(O)OCH₂CH₃]₂, —P(O)[—N(H)CH(CH₃)C(O)OCH₂CH₃][3,4-methylenedioxy-phenyl], —P(O) [—N(H)C(CH₃)₂C(O)OCH₂CH₃][3,4-methylenedioxyphenyl], and —P(O)[—OCH(3-chlorophenyl)CH₂CH₂O—]. In another aspect, X is —PO₃H₂. In yet another aspect, X is selected from the group consisting of —P(O)[—OCH₂OC(O)-t-butyl]₂ and —P(O)[—OCH₂OC(O)-i-propyl]₂.

In one aspect, X is selected from the group consisting of —P(O)[—OCH₂OC(O)O-ethyl]₂ and —P(O)[—OCH₂OC(O)O-i-propyl]₂. In another aspect, X is selected from the group consisting of —P(O)[—N(F)CH(CH₃)C(O)OCH₂CH₃]₂ and —P(O)[—N(H)C(CH₃)₂C(O)OCH₂CH₃]₂. In a further aspect, X is —P(O)[—OCH₂CH₂SC(O)Me]₂. In another aspect, X is selected from the group consisting of —P(O)[—N(H)CH(CH₃)C(O)OCH₂CH₃][3,4-methylenedioxyphenyl] and —P(O)[—N(H)C(CH₃)₂C(O)OCH₂CH₃][3,4-methylenedioxyphenyl]. In a further aspect, X is selected from the group consisting of —PO₃H₂, —P(O)[—OCR^{z}₂OC(O)R^{y}]₂, —P(O)[—OCR^{z}₂OC(O)OR^{y}]₂, —P(O)[—N(H)CR^{z}₂C(O)OR^{y}]₂, —P(O)[—N(H)CR^{z}₂C(O)OR^{y}][—OR¹¹] and —P(O)[—OCH(V)CH₂CH₂O—], wherein V is selected from the group consisting of optionally substituted aryl, aryl, heteroaryl, and optionally substituted heteroaryl. In another aspect, X is selected from the group consisting of —PO₃H₂, —P(O)[—OCH₂OC(O)-t-butyl]₂, —P(O)[—OCH₂OC(O)O-i-propyl]₂, —P(O)[—N(H)CH(CH₃)C(O)OCH₂CH₃]₂, —P(O)[—N(H)C(CH₃)₂C(O)OCH₂CH₃]₂, —P(O)[—N(H)CH(CH₃)C(O)OCH₂CH₃][3,4-methylenedioxyphenyl], —P(O)[—N(H)C(CH₃)₂C(O)OCH₂CH₃][3,4-methylenedioxyphenyl], and —P(O)[—OCH(3-chlorophenyl)CH₂CH₂O—].

In another aspect, X is —P(O)YR¹¹Y′R¹¹

wherein Y and Y′ are each independently selected from —O— and —NR—; together R¹¹ and R¹¹ are the group:

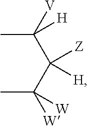

wherein

V, W, and W′ are independently selected from the group consisting of hydrogen, optionally substituted alkyl, optionally substituted aralkyl, heterocycloalkyl, aryl, substituted aryl, heteroaryl, substituted heteroaryl, optionally substituted 1-alkenyl, and optionally substituted 1-alkynyl;

or together V and Z are connected via an additional 3-5 atoms to form a cyclic group containing 5-7 atoms, wherein 0-1 atoms are heteroatoms and the remaining atoms are carbon, substituted with hydroxy, acyloxy, alkylthiocarbonyloxy, alkoxycarbonyloxy, or aryloxycarbonyloxy attached to a carbon atom that is three atoms from both Y groups attached to the phosphorus; or

or together V and Z are connected via an additional 3-5 atoms to form a cyclic group, wherein 0-1 atoms are heteroatoms and the remaining atoms are carbon, that is fused to an aryl group at the beta and gamma position to the Y attached to the phosphorus;

or together V and W are connected via an additional 3 carbon atoms to form an optionally substituted cyclic group containing 6 carbon atoms and substituted with one substituent selected from the group consisting of hydroxy, acyloxy, alkoxycarbonyloxy, alkylthiocarbonyloxy, and aryloxycarbonyloxy, attached to one of said carbon atoms that is three atoms from a Y attached to the phosphorus;

or together Z and W are connected via an additional 3-5 atoms to form a cyclic group, wherein 0-1 atoms are heteroatoms and the remaining atoms are carbon, and V must be aryl, substituted aryl, heteroaryl, or substituted heteroaryl;

or together W and W′ are connected via an additional 2-5 atoms to form a cyclic group, wherein 0-2 atoms are heteroatoms and the remaining atoms are carbon, and V must be aryl, substituted aryl, heteroaryl, or substituted heteroaryl;

Z is selected from the group consisting of —CHR^{z}OH, —CHR^{z}OC(O)R^{y}, —CHR^{z}OC(S)R^{y}, —CHR^{z}OC(S)OR^{y}, —CHR²OC(O)SR^{y}, —CHROCO₂R^{y}, —OR^{z}, —SR^{z}, —CHR^{e}N₃, —CH₂aryl, —CH(aryl)OH, —CH(CH═CR^{z}₂)OH, —CH(C≡CR^{z})OH, —R^{z}, —NR^{z}₂, —OCOR^{y}, —OCO₂R^{y}, —SCOR^{y}, —SCO₂R^{y}, —NHCOR^{e}, —NHCO₂R^{y}, —CH₂NHaryl, —(CH₂)_{q}—OR^{z}, and —(CH₂)_{q}—SR^{z};

q is an integer 2 or 3;

with the provisos that:

a) V, Z, W, W′ are not all —H; and

b) when Z is —R^{z}, then at least one of V, W, and W′ is not —H, alkyl, aralkyl, or heterocycloalkyl;

Each R^{z} is selected from the group consisting of R^{y} and —H;

Each R^{y} is selected from the group consisting of alkyl, aryl, heterocycloalkyl, and aralkyl; and

Each R^{v} is selected from the group consisting of —H, lower alkyl, acyloxyalkyl, alkoxycarbonyloxyalkyl, and lower acyl.

In one aspect, V is optionally substituted aryl. In another aspect, V is selected from the group consisting of 3-chlorophenyl, 4-chlorophenyl, 3-bromophenyl, 3-fluorophenyl, pyrid-4-yl, pyrid-3-yl and 3,5-dichlorophenyl.

In one aspect, the relative stereochemistry between the V-group substituent and T on the dioxaphosphonane ring is cis. In another aspect, the cis dioxaphosphonane ring has R stereochemistry at the carbon where V is attached. In another aspect, the cis dioxaphosphonane ring has S stereochemistry at the carbon where V is attached.

In one aspect R¹¹ is not hydrogen.

In a further aspect when G is —O—, T is —CH₂—, R¹ and R² are each bromo, R³ is iso-propyl, and R⁵ is —OH, then R⁴ is not hydrogen. In another aspect, when G is —O—, T is —(CH₂)₀₋₄, R¹ and R² are independently selected from the group consisting of halogen, alkyl of 1 to 3 carbons, and cycloalkyl of 3 to 5 carbons, R³ is alkyl of 1 to 4 carbons or cycloalkyl of 3 to 7 carbons, and R⁵ is —OH, then R⁴ is not hydrogen; and wherein when G is —O—, R⁵ is selected from the group consisting of

NHC(O)R^{e}, —NHS(═O)₁₋₂R^{e}, —NHC(═S)NH(R^{h}), and —NHC(O)NH(R^{h}), T is selected from the group consisting of —(CH₂)ₘ—, —CH═CH—, —O(CH₂)₁₋₂—, and —NH(CH₂)₁₋₂—, then R⁴ is not hydrogen. In a further aspect for the compounds of Formula I, G is selected from the group consisting of —O— and —CH₂—; T is selected from the group consisting of (CR^{a}₂)ₙ, O(CR^{b}₂)(CR^{a}₂)ₚ—, N(R^{c})(CR^{b}₂)(CR^{a}₂)ₚ—, S(CR^{b}₂)(CR^{a}₂)ₚ, NR^{b}(CO)—, and CH₂CH(NR^{c}R^{b}); R¹ and R² are each independently selected from the group consisting of halogen, —C₁-C₄ alkyl, —CF₃, and cyano; R⁴ is selected from the group consisting of hydrogen, halogen, —C₁-C₄ alkyl, cyano and CF₃; R⁵ is selected from the group consisting of —OH, —OC(O)R^{e}, —OC(O)OR^{h}, —F and —NHC(O)R^{e}; R³ is selected from the group consisting of halogen, optionally substituted —C₁-C₆ alkyl, —CF₃, cyano, —C(O)NR^{f}R^{g}, optionally substituted —(CR^{a}₂)ₙaryl, —SO₂NR^{f}R^{g}, and —SO₂R^{e}; and X is selected from the group consisting of —PO₃H₂, —P(O)[—OCR^{z}₂OC(O)R^{y}]₂, —P(O)[—OCR^{z}₂OC(O)OR^{y}]₂, —P(O)[—Oalk-SC(O)R^{y}]₂, —P(O)[—N(H)CR^{z}₂C(O)OR^{y}]₂, —P(O)[—N(H)CR^{z}₂C(O)OR^{y}][—OR¹¹] and —P(O)[—OCH(V)CH₂CH₂O—], wherein V is selected from the group consisting of optionally substituted aryl, aryl, heteroaryl, and optionally substituted heteroaryl.

In another aspect, G is selected from the group consisting of —O— and —CH₂—; T is selected from the group consisting of (CR^{a}₂)ₙ, O(CR^{b}₂)(CR^{a}₂)ₚ—, N(R^{c})₂)(CR^{b} b 2) (CR^{a}₂)ₚ—, S(CR^{b}₂)(CR^{a}₂)ₚ—, NR^{b}(CO)—, and CH₂CH(NR^{c}R^{b})—; R¹ and R² are each independently selected from the group consisting of halogen, —C₁-C₄ alkyl, —CF₃, and cyano; R⁴ is selected from the group consisting of hydrogen, halogen, —C₁-C₄ alkyl, cyano and CF₃; R¹ is selected from the group consisting of —OH, —OC(O)R^{e}, —OC(O)OR^{h}, —F and —NHC(O)R^{e}; R³ is selected from the group consisting of halogen, optionally substituted —C₁-C₆ alkyl, —CF₃, cyano, —C(O)NR^{f}R^{g}, optionally substituted-(CR^{a}₂)ₙaryl, —SO₂NR^{f}R^{g}, and —SO₂R^{e}; and X is selected from the group consisting of —PO₃H₂, —P(O)[—OCR^{z}₂OC(O)R^{y}]₂, —P(O)[—OCR^{z}₂OC(O)OR^{y}]₂, —P(O)[—N(H)_{c}R^{z}₂C(O)OR^{y}]₂, —P(O)[—N(H)CR^{z}₂C(O)OR^{y}][—OR¹¹] and —P(O)[—OCH(V)CH₂CH₂O—], wherein V is selected from the group consisting of optionally substituted aryl, aryl, heteroaryl, and optionally substituted heteroaryl.

In an additional aspect, G is selected from the group consisting of —O— and —CH₂—; T is —CH₂CH(NH₂)—; R¹ and R² are each independently selected from the group consisting of iodo, bromo, chloro, methyl, and cyano; R⁴ is hydrogen; R⁵ is selected from the group consisting of —OH and —OC(O)R^{e}; R³ is selected from the group consisting of halogen, optionally substituted —C₁-C₆ alkyl, optionally substituted —CH₂aryl, optionally substituted —CH(OH)aryl, —C(O)-amido wherein the amido group is selected from the group consisting of phenethylamino, piperidinyl, 4-methypiperizinyl, morpholinyl, cyclohexylamino, anilinyl, and indolinyl, —S(═O)₂-amido wherein the amido group is selected from the group consisting of phenethylamino, piperidinyl, 4-methypiperizinyl, morpholinyl, cyclohexylamino, anilinyl, and indolinyl, and —SO₂R wherein R is selected from the group consisting of phenyl, 4-chlorophenyl, 4-fluorophenyl, and 4-pyridyl, and X is selected from the group consisting of —PO₃H₂, —P(O)[—OCR^{z}₂OC(O)R^{y}]₂, —P(O)[—OCR^{z}₂OC(O)OR^{y}]₂, —P(O)[—N(H)CR^{z}₂C(O)OR^{y}]₂, —P(O)[—N(H)CR^{z}₂C(O)OR^{y}][OR^{e}] and —P(O) [—OCR^{z}(aryl)CH₂CH₂O—].

In another aspect, when G is —O—, T is —CH₂—, R¹ and R² are bromo, R³ is iso-propyl, R⁵ is —OH, and X is selected from the group consisting of —PO₃H₂, —P(O)[—OCR^{z}₂OC(O)R^{y}]₂, —P(O)[—OCR^{z}₂OC(O)OR^{y}]₂, —P(O)[—N(H)CR^{z}₂C(O)OR^{y}]₂, —P(O)[—N(H)CR^{e}₂C(O)OR^{y}][OR^{e}] and —P(O)[—OCR^{z}(aryl)CH₂CH₂O—], then R⁴ is not hydrogen.

In one aspect for the compounds of Formula I, G is —O—; T is —CH₂CH(NH₂)—; R¹ and R² are each iodo; R⁴ is selected from the group consisting of hydrogen and iodo; R⁵ is —OH; and R³ is iodo; and X is selected from the group consisting of —PO₃H₂, —P(O) [—OCR^{z}₂OC(O)R^{y}]₂, —P(O)[—OCR^{z}₂OC(O)OR^{y}]₂, —P(O)[—N(H)CR^{z}₂C(O)OR^{y}]₂, —P(O)[—N(H)CR^{z}₂C(O)OR^{y}][OR^{e}] and —P(O)[—OCR^{z}(aryl)CH₂CH₂O—].

In another aspect G is —O—; T is —CH₂CH(NH₂)—; R¹ and R² are each iodo; R⁴ is selected from the group consisting of hydrogen and iodo; R⁵ is —OH; R³ is iodo; and X is selected from the group consisting of —PO₃H₂, —P(O)[—OCH₂OC(O)-t-butyl]₂, P(O) [—OCH₂OC(O)O-i-propyl]₂, —P(O)[—N(H)CH(CH₃)C(O)OCH₂CH₃]₂, —P(O)[—N(H)C(CH₃)₂C(O)OCH₂CH₃]₂, —P(O)[—N(H)CH(CH₃)C(O)OCH₂CH₃][3,4-methylenedioxyphenyl], —P(O)[—N(H)C(CH₃)₂C(O)OCH₂CH₃][3,4-methylenedioxyphenyl], and —P(O)[—OCH(3-chlorophenyl)CH₂CH₂O—].

In a further aspect for compounds of Formula I, G is selected from the group consisting of —O— and —CH₂—; T is —N(H)C(O)—; R¹ and R² are each independently selected from the group consisting of iodo, bromo, chloro, methyl, and cyano; R⁴ is selected from the group consisting of hydrogen, iodo, 4-chlorophenyl, and cyclohexyl; R⁵ is selected from the group consisting of —OH and —OC(O)R^{e}; R³ is selected from the group consisting of hydrogen, iodo, bromo, optionally substituted —C₁-C₆ alkyl, optionally substituted —CH₂aryl, optionally substituted —CH(OH)aryl, —C(O)-amido wherein the amido group is selected from the group consisting of phenethylamino, piperidinyl, 4-methypiperizinyl, morpholinyl, cyclohexylamino, anilinyl, and indolinyl, —S(═O)₂-amido wherein the amido group is selected from the group consisting of phenethylamino, piperidinyl, 4-methypiperizinyl, morpholinyl, cyclohexylamino, anilinyl, and indolinyl, and —SO₂R wherein R is selected from the group consisting of phenyl, 4-chlorophenyl, 4-fluorophenyl, and 4-pyridyl; and X is selected from the group consisting of —PO₃H₂, —P(O)[—OCR^{z}₂OC(O)R^{y}]₂, —P(O)[—OCR^{z}₂OC(O)OR^{y}]₂, —P(O)[—N(H)CR^{z}₂C(O)OR^{y}]₂, —P(O)[—N(H)CR^{z}₂C(O)OR^{y}][OR^{e}] and —P(O)[—OCR^{z}(aryl)CH₂CH₂O—].

An additional aspect is when G is —O—; T is —N(H)C(O)—; R¹ and R² are methyl; R⁴ is hydrogen; R⁵ is —OH; R³ is —CH(OH)(4-fluorophenyl); and X is selected from the group consisting of —PO₃H₂, —P(O)[—OCR^{z}₂OC(O)R^{y}]₂, —P(O) [—OCR^{z}₂OC(O)OR^{y}]₂, —P(O)[—N(H)CR^{z}₂C(O)OR^{y}]₂, —P(O)[—N(H)CR^{z}₂C(O)OR^{y}][OR^{e}] and —P(O)[—OCR^{z}(aryl)CH₂CH₂O—].

In an additional aspect, G is —O—; T is —N(H)C(O)—; R¹ and R² are each methyl; R⁴ is hydrogen; R⁵ is —OH; R³ is —CH(OH)(4-fluorophenyl); and X is selected from the group consisting of —PO₃H₂, —P(O)[—OCH₂OC(O)-t-butyl]₂, —P(O)[—OCH₂OC (O)O-i-propyl]₂, —P(O)[—N(H)CH(CH₃)C(O)OCH₂CH₃]₂, —P(O)[—N(H)C(CH₃)₂C(O)OCH₂CH₃]₂, —P(O)[—N(H)CH(CH₃)C(O)OCH₂CH₃][3,4-methylenedioxyphenyl], —P(O) [—N(H)C(CH₃)₂C(O)OCH₂CH₃][₃,4-methylenedioxyphenyl], and —P(O) [—OCH(3-chlorophenyl)CH₂CH₂O—].

In a further aspect G is selected from the group consisting of —O— and —CH₂—; T is —OCH₂—; R¹ and R² are each independently selected from the group consisting of iodo, bromo, chloro, methyl, and cyano; R⁴ is selected from the group consisting of hydrogen, iodo, 4-chlorophenyl, and cyclohexyl; R⁵ is selected from the group consisting of —OH and —OC(O)R^{e}; R³ is selected from the group consisting of hydrogen, iodo, bromo, optionally substituted lower alkyl, optionally substituted —CH₂aryl, optionally substituted —CH(OH)aryl, —C(O)-amido wherein the amido group is selected from the group consisting of phenethylamino, piperidinyl, 4-methypiperizinyl, morpholinyl, cyclohexylamino, anilinyl, and indolinyl, —S(═O)₂-amido wherein the amido group is selected from the group consisting of phenethylamino, piperidinyl, 4-methypiperizinyl, morpholinyl, cyclohexylamino, anilinyl, and indolinyl, and —SO₂R wherein R is selected from the group consisting of phenyl, 4-chlorophenyl, 4-fluorophenyl, and 4-pyridyl; and X is selected from the group consisting of —PO₃H₂, —P(O)[—OCR^{z}₂OC(O)R^{y}]₂, —P(O)[—OCR^{z}₂OC(O)OR^{y}]₂, P(O)[—N(H)CR^{z}₂C(O)OR^{y}]₂, —P(O)[—N(H)CR^{z}₂C(O)OR^{y}][OR^{e}] and —P(O)[—OCR^{z}(aryl)CH₂CH₂O—].

In another aspect G is —CH₂—; T is —OCH₂—; R¹ and R² are each methyl; R⁴ is hydrogen; R⁵ is —OH; R³ is iso-propyl; and X is selected from the group consisting of —PO₃H₂, —P(O)[—OCR^{z}₂OC(O)R^{y}]₂, —P(O)[—OCR^{a}₂OC(O)OR^{y}]₂, —P(O)[—N(H)CR^{z}₂C(O)OR^{y}]₂, —P(O)[—N(H)CR^{z}₂C(O)OR^{y}][OR^{e}] and —P(O)[—OCR^{z}(aryl)CH₂CH₂O—].

In another aspect, G is —CH₂—; T is —OCH₂—; R¹ and R² are each methyl; R⁴ is hydrogen; R⁵ is —OH; R³ is iso-propyl; and X is selected from the group consisting of —PO₃H₂, —P(O)[—OCH₂OC(O)-t-butyl]₂, —P(O)[—OCH₂OC(O)O-i-propyl]₂, —P(O)[—N(H)CH(CH₃)C(O)OCH₂CH₃]₂, —P(O)[—N(H)C(CH₃)₂C(O)OCH₂CH₃]₂, —P(O)[—N(H)CH(CH₃)C(O)OCH₂CH₃][3,4-methylenedioxyphenyl], —P(O)[—N(H)C(CH₃)₂C(O)OCH₂CH₃][3,4-methylenedioxyphenyl], and —P(O)[—OCH(3-chlorophenyl)CH₂CH₂O—].

In a further aspect, G is selected from the group consisting of —O— and —CH₂—; T is —CH₂—; R¹ and R² are each independently selected from the group consisting of iodo, bromo, chloro, methyl, and cyano; R⁴ is selected from the group consisting of hydrogen, iodo, 4-chlorophenyl, and cyclohexyl; R⁵ is selected from the group consisting of —OH and —OC(O)R^{e}; R³ is selected from the group consisting of hydrogen, iodo, bromo, optionally substituted lower alkyl, optionally substituted —CH₂aryl, optionally substituted —CH(OH)aryl, —C(O)-amido wherein the amido group is selected from the group consisting of phenethylamino, piperidinyl, 4-methypiperizinyl, morpholinyl, cyclohexylamino, anilinyl, and indolinyl, —S(═O)₂-amido wherein the amido group is selected from the group consisting of phenethylamino, piperidinyl, 4-methypiperizinyl, morpholinyl, cyclohexylamino, anilinyl, and indolinyl, and —SO₂R wherein R is selected from the group consisting of phenyl, 4-chlorophenyl, 4-fluorophenyl, and 4-pyridyl; and X is selected from the group consisting of —PO₃H₂, —P(O)[—OCR^{z}₂OC(O)R^{y}]₂, —P(O) [—OCR^{z}₂OC(O)OR^{y}]₂, —P(O)[—N(H)CR^{z}₂C(O)OR^{y}]₂, —P(O)[—N(H)CR^{z}₂C(O)OR^{y}][OR^{e}] and —P(O)[—OCR^{z}(aryl)CH₂CH₂O—].

In additional aspects, when G is —O—, T is —CH₂—, R¹ and R² are each bromo, R³ is iso-propyl, R⁵ is —OH; and X is selected from the group consisting of —PO₃H₂, —P(O)[—OCR^{z}₂OC(O)R^{y}]₂, —P(O)[—OCR^{z}₂OC(O)OR^{y}]₂, —P(O)[—N(H)CR^{z}₂C(O)OR^{y}]₂, —P(O)[—N(H)CR^{z}₂C(O)OR^{y}][OR^{e}] and —P(O)[—OCR^{z}(aryl)CH₂CH₂O—], then R⁴ is not hydrogen.

In another aspect, G is —O—; T is —CH₂—; R¹ and R² are each chloro; R⁴ is hydrogen; R⁵ is —OH; R³ is i-propyl; and X is selected from the group consisting of —PO₃H₂, —P(O) [—OCR^{z}₂OC(O)R^{y}]₂, —P(O)[—OCR^{z}₂OC(O)OR^{y}]₂, —P(O)[—N(H)CR^{z}₂C(O)OR^{y}]₂, —P(O) [—N(H)CR^{z}₂C(O)OR^{y}][OR^{e}] and —P(O)[—OCR^{z}(aryl)CH₂CH₂O—].

In another aspect, G is —O—; T is —CH₂—; R¹ and R² are each chloro; R⁴ is hydrogen; R⁵ is —OH; R³ is i-propyl; and X is selected from the group consisting of —PO₃H₂, —P(O)[—OCH₂OC(O)-t-butyl]₂, —P(O)[—OCH₂OC(O)O-i-propyl]₂, —P(O)[—N(H)CH(CH₃)C(O)OCH₂CH₃]₂, —P(O)[—N(H)C(CH₃)₂C(O)OCH₂CH₃]₂, —P(O)[—N(H)CH(CH₃)C(O)OCH₂CH₃][3,4-methylenedioxyphenyl], —P(O) [—N(H)C(CH₃)₂C(O)OCH₂CH₃][3,4-methylenedioxyphenyl], and —P(O)[—OCH(3-chlorophenyl)CH₂CH₂O—].

In additional aspects for compounds of Formula I, G is selected from the group consisting of —O— and —CH₂—; T is —CH₂CH₂—; R¹ and R² are each independently selected from the group consisting of iodo, bromo, chloro, methyl, and cyano; R⁴ is selected from the group consisting of hydrogen, iodo, 4-chlorophenyl, and cyclohexyl; R⁵ is selected from the group consisting of —OH and —OC(O)R^{e}; R³ is selected from the group consisting of hydrogen, iodo, bromo, optionally substituted lower alkyl, optionally substituted —CH₂aryl, optionally substituted —CH(OH)aryl, —C(O)-amido wherein the amido group is selected from the group consisting of phenethylamino, piperidinyl, 4-methypiperizinyl, morpholinyl, cyclohexylamino, anilinyl, and indolinyl, —S(═O)₂-amido wherein the amido group is selected from the group consisting of phenethylamino, piperidinyl, 4-methypiperizinyl, morpholinyl, cyclohexylamino, anilinyl, and indolinyl, and —SO₂R wherein R is selected from the group consisting of phenyl, 4-chlorophenyl, 4-fluorophenyl, and 4-pyridyl; and X is selected from the group consisting of —PO₃H₂, —P(O)[—OCR^{z}₂OC(O)R^{y}]₂, —P(O)[—OCR^{z}₂OC(O)OR^{y}]₂, —P(O)[—N(H)CR^{z}₂C(O)OR^{y}]₂, —P(O) [—N(H)CR^{z}₂C(O)OR^{y}][OR^{e}] and —P(O) [—OCR^{z}(aryl)CH₂CH₂O—].

In a further aspect, G is —O—; T is —CH₂CH₂—; R¹ and R² are each chloro; R⁴ is hydrogen; R⁵ is —OH; R³ is iso-propyl; and X is selected from the group consisting of —PO₃H₂, —P(O)[—OCR^{z}₂OC(O)R^{y}]₂, —P(O)[—OCR^{z}₂OC(O)OR^{y}]₂, —P(O)[—N(H)CR^{z}₂C(O)OR^{y}]₂, —P(O)[—N(H)CR^{z}₂C(O)OR^{y}][OR^{e}] and —P(O)[—OCR^{z}(aryl)CH₂CH₂O—].

In another aspect, G is —O—; T is —CH₂CH₂—; R¹ and R² are each chloro; R⁴ is hydrogen; R⁵ is —OH; R³ is iso-propyl; and X is selected from the group consisting of —PO₃H₂, —P(O)[—OCH₂OC(O)-t-butyl]₂, —P(O)[—OCH₂OC(O)O-i-propyl]₂, —P(O)[—N(H)CH(CH₃)C(O)OCH₂CH₃]₂, —P(O)[—N(H)C(CH₃)₂C(O)OCH₂CH₃]₂, —P(O)[—N(H)CH(CH₃)C(O)OCH₂CH₃][3,4-methylenedioxyphenyl], —P(O)[—N(H)C(CH₃)₂C(O)OCH₂CH₃][3,4-methylenedioxyphenyl], and —P(O)[—OCH(3-chlorophenyl)CH₂CH₂O—].

In an additional aspect, G is —CH₂—; T is —OCH₂—; R¹ and R² are each methyl; R⁴ is hydrogen; R⁵ is —OH; R³ is iso-propyl; and X is —PO₃H₂. In a further aspect, G is —CH₂—; T is —OCH₂—; R¹ and R² are each methyl; R⁴ is hydrogen; R⁵ is —OH; R³ is iso-propyl; and X is selected from the group consisting of —P(O)[—OCH₂OC(O)-t-butyl]₂ and —P(O)[—OCH₂OC(O)-i-propyl]₂. In another aspect, G is —CH₂—; T is —OCH₂—; R¹ and R² are each methyl; R⁴ is hydrogen; R⁵ is —OH; R³ is iso-propyl; and X is selected from the group consisting of —P(O)[—OCH₂OC(O)O-ethyl]₂ and —P(O)[—OCH₂OC(O)O-i-propyl]₂. In an additional aspect, G is —CH₂—; T is —OCH₂—; R¹ and R² are each methyl; R⁴ is hydrogen; R⁵ is —OH; R³ is iso-propyl; and X is selected from the group consisting of —P(O)[—N(H)CH(CH₃)C(O)OCH₂CH₃]₂ and —P(O)[—N(H)C(CH₃)₂C (O)OCH₂CH₃]₂. In additional aspects, G is —CH₂—; T is —OCH₂—; R¹ and R² are methyl; R⁴ is hydrogen; R⁵ is —OH; R³ is iso-propyl; and X is —P(O)[—OCH₂CH₂SC(O)Me]₂, or X is —P(O)[—N(H)C(CH₃)C(O)OCH₂CH₃][3,4-methylenedioxyphenyl], or X is —P(O)[—N(H)C(CH₃)₂C(O)OCH₂CH₃][3,4-methylenedioxyphenyl].

In another aspect, G is —O—, T is —(CH₂)₀₋₄—, R¹ and R² are independently selected from the group consisting of hydrogen, halogen, alkyl of 1 to 3 carbons, and cycloalkyl of 3 to 5 carbons, R³ is alkyl of 1 to 4 carbons or cycloalkyl of 3 to 7 carbons, and R⁵ is —OH, then R⁴ is not hydrogen; and wherein when G is —O—, R⁵ is selected from the group consisting of NHC(O)R^{e}, —NHS(═O)₁₋₂R^{e}, —NHC(S)NH(Th), and —NHC(O)NH(R^{h}), T is selected from the group consisting of —(CH₂)ₘ—, —CH═CH—, —O(CH₂)₁₋₂—, and —NH(CH₂)₁₋₂—, then R⁴ is not hydrogen.

In an additional aspect, G is —CH₂—; T is —OCH₂—; R¹ and R² are each methyl; R¹ is hydrogen; R⁵ is —OH; R³ is iso-propyl; and X is —P(O)YR¹¹Y′R¹¹;

wherein Y and Y′ are each independently selected from —O— and —NR^{v}—; together R¹¹ and R¹¹ are the group:

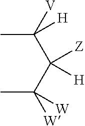

wherein:

V, W, and W′ are independently selected from the group consisting of hydrogen, optionally substituted alkyl, optionally substituted aralkyl, heterocycloalkyl, aryl, substituted aryl, heteroaryl, substituted heteroaryl, optionally substituted 1-alkenyl, and optionally substituted 1-alkynyl;

or together V and Z are connected via an additional 3-5 atoms to form a cyclic group containing 5-7 atoms, wherein 0-1 atoms are heteroatoms and the remaining atoms are carbon, substituted with hydroxy, acyloxy, alkoxycarbonyloxy, or aryloxycarbonyloxy attached to a carbon atom that is three atoms from both Y groups attached to the phosphorus; or

or together V and Z are connected via an additional 3-5 atoms to form a cyclic group, wherein 0-1 atoms are heteroatoms and the remaining atoms are carbon, that is fused to an aryl group at the beta and gamma position to the Y attached to the phosphorus;

or together V and W are connected via an additional 3 carbon atoms to form an optionally substituted cyclic group containing 6 carbon atoms and substituted with one substituent selected from the group consisting of hydroxy, acyloxy, alkoxycarbonyloxy, alkylthiocarbonyloxy, and aryloxycarbonyloxy, attached to one of said carbon atoms that is three atoms from a Y attached to the phosphorus;

or together Z and W are connected via an additional 3-5 atoms to form a cyclic group, wherein 0-1 atoms are heteroatoms and the remaining atoms are carbon, and V must be aryl, substituted aryl, heteroaryl, or substituted heteroaryl;

or together W and W′ are connected via an additional 2-5 atoms to form a cyclic group, wherein 0-2 atoms are heteroatoms and the remaining atoms are carbon, and V must be aryl, substituted aryl, heteroaryl, or substituted heteroaryl;

Z is selected from the group consisting of —CHR^{z}OH, —CHR^{z}OC(O)R^{y}, —CHR^{z}OC(S)R^{y}, —CHR^{z}OC(S)OR^{y}, —CHR^{z}OC(O)SR^{y}, —CHR^{z}OCO₂R^{y}, —OR^{z}, —SR^{z}, —CHR^{z}N₃, —CH₂aryl, —CH(aryl)OH, —CH(CH═CR^{z}₂)OH, —CH(C≡CR^{z})OH, —R^{z}, —NR^{z}₂, —OCOR^{y}, —OCO₂R^{y}, —SCOR^{y}, —SCO₂R^{y}, —NHCOR^{z}, —NHCO₂R^{y}, —CH₂NHaryl, —(CH₂)_{q}—OR^{z}, and —(CH₂)_{q}—SR^{z};

q is an integer 2 or 3;

with the provisos that:

a) V, Z, W, W′ are not all —H; and

b) when Z is —R^{z}, then at least one of V, W, and W′ is not —H, alkyl, aralkyl, or heterocycloalkyl;

Each R^{z} is selected from the group consisting of R^{y} and —H;

Each R^{y} is selected from the group consisting of alkyl, aryl, heterocycloalkyl, and aralkyl;

Each R^{x} is independently selected from the group consisting of —H, and alkyl, or together R^{x} and R^{x} form a cyclic alkyl group;

Each R^{v} is selected from the group consisting of —H, lower alkyl, acyloxyalkyl, alkoxycarbonyloxyalkyl, and lower acyl. In a further aspect V is aryl. In an additional aspect Z is hydrogen, W is hydrogen, and W′ is hydrogen. In an additional aspect, V is 3-chlorophenyl, 4-chlorophenyl, 3-bromophenyl, 3-fluorophenyl, pyrid-4-yl, pyrid-3-yl or 3,5-dichlorophenyl. In a further aspect the relative stereochemistry between the substituents on the dioxaphosphonane ring is cis.

In another aspect, each R^{a} is independently selected from the group consisting of hydrogen, optionally substituted —C₁-C₂ alkyl, halogen, —OH, optionally substituted —O—C₁-C₂ alkyl, —OCF₃, optionally substituted —S—C₁-C₂ alkyl, —NR^{b}R^{c}, optionally substituted —C₂ alkenyl, and optionally substituted —C₂ alkynyl;

Each R^{b} is independently selected from the group consisting of hydrogen, optionally substituted —C₁-C₂ alkyl;

Each R^{c} is independently selected from the group consisting of hydrogen, optionally substituted —C₁-C₄ alkyl, and optionally substituted —C(O)—C₁-C₂ alkyl, —C(O)H;

Each R^{d} is selected from the group consisting of optionally substituted —C₁-C₆ alkyl, optionally substituted —C₂-C₆ alkenyl, optionally substituted —C₂-C₆ alkynyl, optionally substituted —(CR^{b}₂)ₙphenyl, optionally substituted —(CR^{b}₂)ₙnonocyclic-heteroaryl, optionally substituted —(CR^{b}₂)ₙ—C₃-C₆-cycloalkyl, optionally substituted —(CR^{b}₂)ₙ—C₄-C₅-heterocycloalkyl, and —C(O)NR^{f}R^{g};

Each R^{e} is selected from the group consisting of optionally substituted —C₁-C₆ alkyl, optionally substituted —C₂-C₆ alkenyl, optionally substituted —C₂-C₆ alkynyl, optionally substituted —(CR^{b}₂)ₙphenyl, optionally substituted —(CR^{b}₂)ₙmonocyclic-heteroaryl, optionally substituted —(CR^{b}₂)ₙ—C₃-C₆-cycloalkyl, optionally substituted —(CR^{b}₂)ₙ—C₄-C₅-heterocycloalkyl;

R^{f} and R^{g} are each independently selected from the group consisting of hydrogen, optionally substituted —C₁-C₆ alkyl, optionally substituted —C₂-C₆ alkenyl, optionally substituted —C₂-C₆ alkynyl, optionally substituted —(CR^{b}₂)ₙphenyl, optionally substituted —(CR^{b}₂)ₙmonocyclic-heteroaryl, optionally substituted —(CR^{b}₂)ₙ—C₃-C₆-cycloalkyl, optionally substituted —(CR^{b}₂)ₙ—C₄-C₅-heterocycloalkyl, or R^{f} and R^{g} may together form an optionally substituted heterocyclic ring, which may contain a second heterogroup selected from the group of O, NR^{b}, and S, wherein said optionally substituted heterocyclic ring may be substituted with 0-2 substituents selected from the group consisting of optionally substituted —C₁-C₂ alkyl, —OR^{b}, oxo, cyano, —CF₃, optionally substituted phenyl, and —C(O)OR^{h};

Each R^{h} is optionally substituted —C₁-C₁₆ alkyl, optionally substituted —C₂-C₁₆ alkenyl, optionally substituted —C₂-C₁₆ alkynyl, optionally substituted —(CR^{b}₂)ₙphenyl, optionally substituted —(CR^{b}₂)ₙmonocyclic-heteroaryl, optionally substituted —(CR^{b}₂)ₙ, —C₃-C₆-cycloalkyl, optionally substituted —CR^{b}₂)ₙ—C₄-C₅-heterocycloalkyl.

In a further aspect, each R^{a} is independently selected from the group consisting of hydrogen, methyl, fluoro, chloro, —OH, —O—CH₃, —OCF₃, —SCH₃, —NHCH₃, —N(CH₃)₂;

Each R^{b} is independently selected from the group consisting of hydrogen, and methyl;

Each R^{c} is independently selected from the group consisting of hydrogen, methyl, —C(O)CH₃, —C(O)H;

Each R^{d} is selected from the group consisting of optionally substituted —C₁-C₄ alkyl, optionally substituted —C₂-C₄ alkenyl, optionally substituted —C₂-C₄ alkynyl, optionally substituted —(CH₂)ₙphenyl, optionally substituted —(CH₂)ₙmonocyclic-heteroaryl, optionally substituted —(CH₂)ₙ—C₃-C₆-cycloalkyl, optionally substituted-(CH₂)ₙ—C₄-C₅-heterocycloalkyl, and —C(O)NR^{f}R^{g};

Each R^{e} is selected from the group consisting of optionally substituted —C₁-C₄ alkyl, optionally substituted —C₂-C₄ alkenyl, optionally substituted —C₂-C₄ alkynyl, optionally substituted —(CH₂)ₙphenyl, optionally substituted —(CH₂)ₙmonocyclic-heteroaryl, optionally substituted —(CH₂)—C₃-C₆-Cycloalkyl, optionally substituted-(CH₂)ₙ, —C₄-C₅-heterocycloalkyl;

R^{f} and R^{g} are each independently selected from the group consisting of hydrogen, optionally substituted —C₁-C₄ alkyl, optionally substituted —C₂-C₄ alkenyl, optionally substituted —C₂-C₄ alkynyl, optionally substituted —(CH₂)ₙphenyl, optionally substituted —CH₂)ₙmonocyclic-heteroaryl, optionally substituted —(CH₂)ₙ—C₃-C₆-cycloalkyl, optionally substituted —(CH₂)ₙ—C₄-C₈-heterocycloalkyl, or R^{f} and R^{g} may together form an optionally substituted heterocyclic ring, which may contain a second heterogroup selected from the group of O, NR^{b}, and S, wherein said optionally substituted heterocyclic ring may be substituted with 0-2 substituents selected from the group consisting of optionally substituted methyl, —OR^{b}, oxo, cyano, —CF₃, optionally substituted phenyl, and —C(O)OR^{h};

Each R^{h} is optionally substituted —C₁-C₄alkyl, optionally substituted —C₂-C₄ alkenyl, optionally substituted —C₂-C₄ alkynyl, optionally substituted —(CH₂), phenyl, optionally substituted —CH₂)ₙmonocyclic-heteroaryl, optionally substituted —(CH₂)ₙ—C₃-C₆-cycloalkyl, optionally substituted —(CH₂)₆—C₄-C₈-heterocycloalkyl.

Exemplary compounds for incorporation into compositions for administration within the methods of the present disclosure include, but are not limited to, those disclosed in U.S. Pat. No. 7,829,552, which is incorporated herein by reference in its entirety. U.S. Pat. No. 7,829,552 further discloses methods of synthesizing said compounds. Such compounds include those having the following structures or pharmaceutically acceptable salts thereof:

| Structure | Compound Number |
|---|---|
| 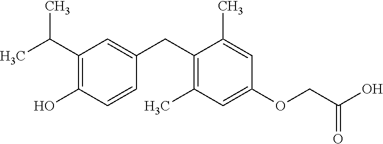 [SMILES: Cc1cc(OCC(=O)O)cc(C)c1Cc1ccc(O)c(C(C)C)c1] | 17 |
| 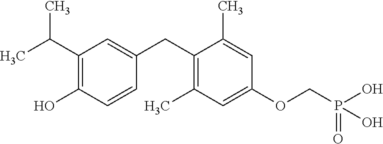 [SMILES: Cc1cc(OCP(=O)(O)O)cc(C)c1Cc1ccc(O)c(C(C)C)c1] | 7 |
| 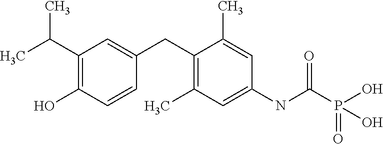 [SMILES: Cc1cc(NC(=O)P(=O)(O)O)cc(C)c1Cc1ccc(O)c(C(C)C)c1] | 1a |
| 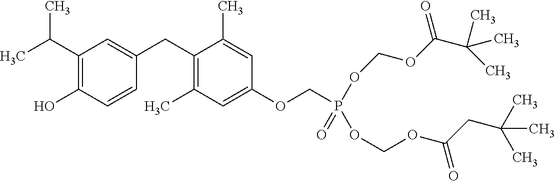 [SMILES: Cc1cc(OCP(=O)(OCOC(=O)CC(C)(C)C)OCOC(=O)C(C)(C)C)cc(C)c1Cc1ccc(O)c(C(C)C)c1] | 12-1 |
| 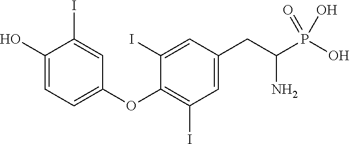 [SMILES: NC(Cc1cc(I)c(Oc2ccc(O)c(I)c2)c(I)c1)P(=O)(O)O] | 2a |
| 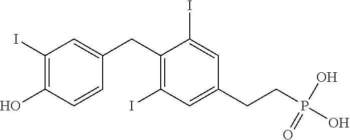 [SMILES: O=P(O)(O)CCc1cc(I)c(Cc2ccc(O)c(I)c2)c(I)c1] | 3a |
| 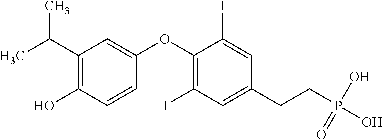 [SMILES: CC(C)c1cc(Oc2c(I)cc(CCP(=O)(O)O)cc2I)ccc1O] | 4a |
| 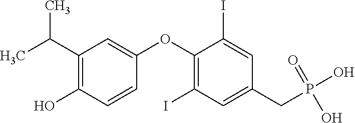 [SMILES: CC(C)c1cc(Oc2c(I)cc(CP(=O)(O)O)cc2I)ccc1O] | 5 |
| 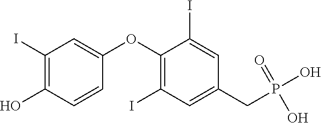 [SMILES: O=P(O)(O)Cc1cc(I)c(Oc2ccc(O)c(I)c2)c(I)c1] | 6 |
| 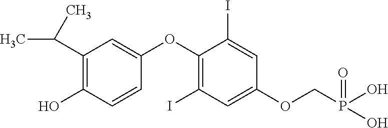 [SMILES: CC(C)c1cc(Oc2c(I)cc(OCP(=O)(O)O)cc2I)ccc1O] | 8 |
| 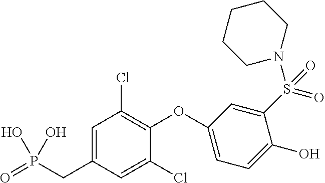 [SMILES: O=P(O)(O)Cc1cc(Cl)c(Oc2ccc(O)c(S(=O)(=O)N3CCCCC3)c2)c(Cl)c1] | 9 |
| 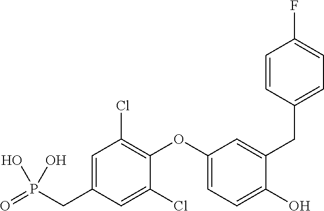 [SMILES: O=P(O)(O)Cc1cc(Cl)c(Oc2ccc(O)c(Cc3ccc(F)cc3)c2)c(Cl)c1] | 11 |
| 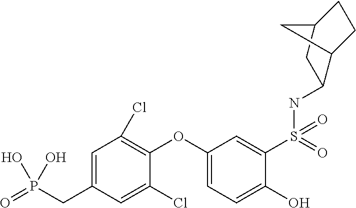 [SMILES: O=P(O)(O)Cc1cc(Cl)c(Oc2ccc(O)c(S(=O)(=O)NC3CC4CCC3C4)c2)c(Cl)c1] | 10 |
| 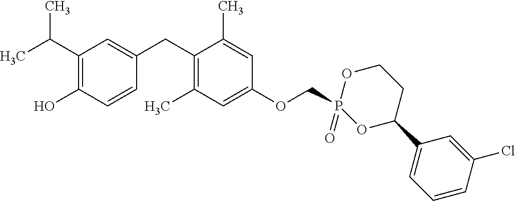 [SMILES: Cc1cc(OC[P@@]2(=O)OCC[C@@H](c3cccc(Cl)c3)O2)cc(C)c1Cc1ccc(O)c(C(C)C)c1] | cis-13-1 |
| 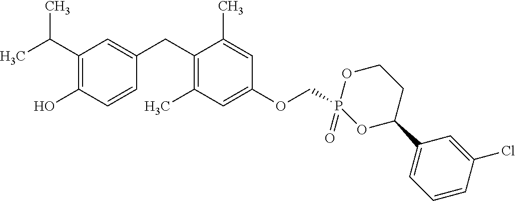 [SMILES: Cc1cc(OC[P@]2(=O)OCC[C@@H](c3cccc(Cl)c3)O2)cc(C)c1Cc1ccc(O)c(C(C)C)c1] | trans-13-1 |
| 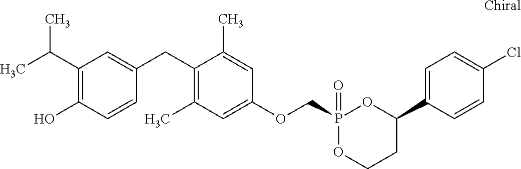 [SMILES: "Chiral" ; Cc1cc(OC[P@]2(=O)OCC[C@H](c3ccc(Cl)cc3)O2)cc(C)c1Cc1ccc(O)c(C(C)C)c1] | cis-13-6 |
| 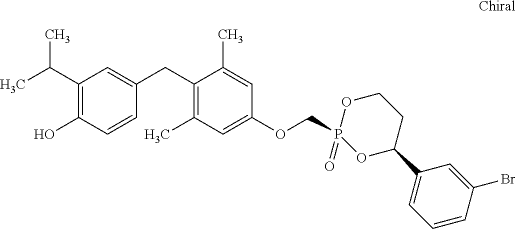 [SMILES: "Chiral" ; Cc1cc(OC[P@@]2(=O)OCC[C@@H](c3cccc(Br)c3)O2)cc(C)c1Cc1ccc(O)c(C(C)C)c1] | cis-13-2 |
| 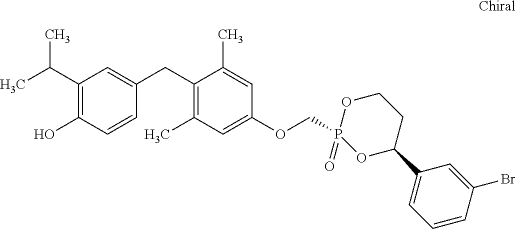 [SMILES: "Chiral" ; Cc1cc(OC[P@]2(=O)OCC[C@@H](c3cccc(Br)c3)O2)cc(C)c1Cc1ccc(O)c(C(C)C)c1] | trans-13-2 |
| 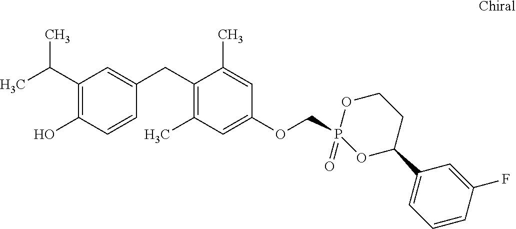 [SMILES: "Chiral" ; Cc1cc(OC[P@@]2(=O)OCC[C@@H](c3cccc(F)c3)O2)cc(C)c1Cc1ccc(O)c(C(C)C)c1] | cis-13-3 |
| 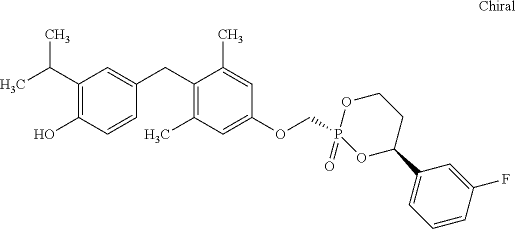 [SMILES: "Chiral" ; Cc1cc(OC[P@]2(=O)OCC[C@@H](c3cccc(F)c3)O2)cc(C)c1Cc1ccc(O)c(C(C)C)c1] | trans-13-3 |
| 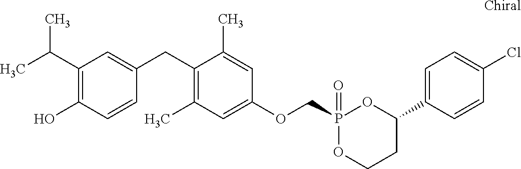 [SMILES: "Chiral" ; Cc1cc(OC[P@]2(=O)OCC[C@@H](c3ccc(Cl)cc3)O2)cc(C)c1Cc1ccc(O)c(C(C)C)c1] | trans-13-6 |
| 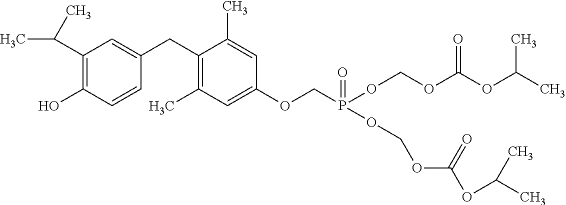 [SMILES: Cc1cc(OCP(=O)(OCOC(=O)OC(C)C)OCOC(=O)OC(C)C)cc(C)c1Cc1ccc(O)c(C(C)C)c1] | 12-3 |
| 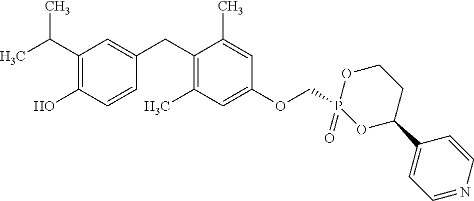 [SMILES: Cc1cc(OC[P@]2(=O)OCC[C@@H](c3ccncc3)O2)cc(C)c1Cc1ccc(O)c(C(C)C)c1] | trans-13-5 |
| 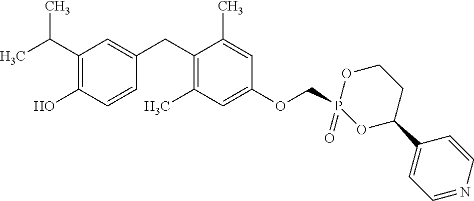 [SMILES: Cc1cc(OC[P@@]2(=O)OCC[C@@H](c3ccncc3)O2)cc(C)c1Cc1ccc(O)c(C(C)C)c1] | cis-13-5 |
| 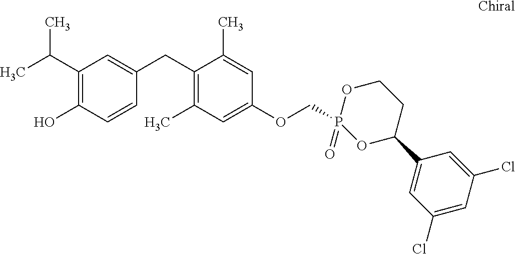 [SMILES: "Chiral" ; Cc1cc(OC[P@]2(=O)OCC[C@@H](c3cc(Cl)cc(Cl)c3)O2)cc(C)c1Cc1ccc(O)c(C(C)C)c1] | trans-13-7 |
| 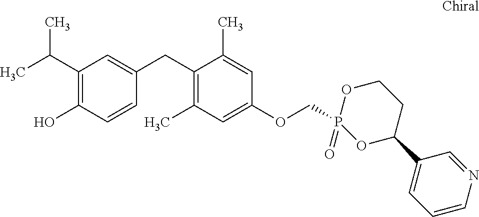 [SMILES: "Chiral" ; Cc1cc(OC[P@]2(=O)OCC[C@@H](c3cccnc3)O2)cc(C)c1Cc1ccc(O)c(C(C)C)c1] | trans-13-4 |
| 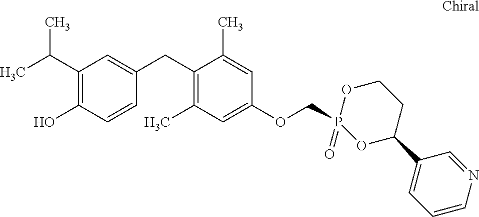 [SMILES: "Chiral" ; Cc1cc(OC[P@@]2(=O)OCC[C@@H](c3cccnc3)O2)cc(C)c1Cc1ccc(O)c(C(C)C)c1] | cis-13-4 |
| 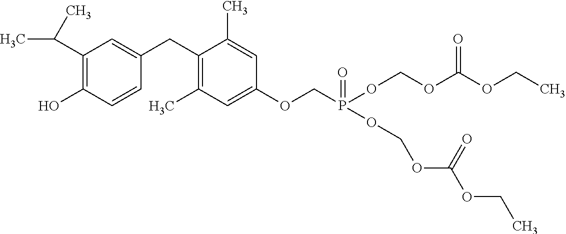 [SMILES: CCOC(=O)OCOP(=O)(COc1cc(C)c(Cc2ccc(O)c(C(C)C)c2)c(C)c1)OCOC(=O)OCC] | 12-2 |
| 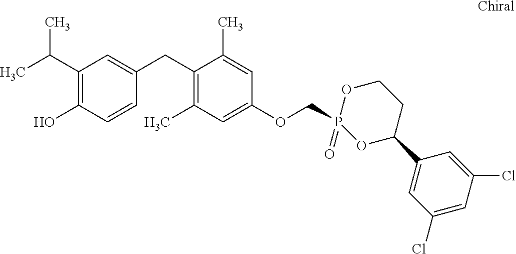 [SMILES: "Chiral" ; Cc1cc(OC[P@@]2(=O)OCC[C@@H](c3cc(Cl)cc(Cl)c3)O2)cc(C)c1Cc1ccc(O)c(C(C)C)c1] | cis-13-7 |
| 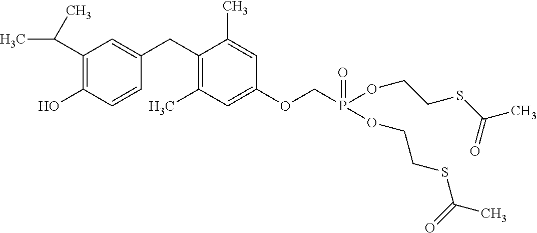 [SMILES: CC(=O)SCCOP(=O)(COc1cc(C)c(Cc2ccc(O)c(C(C)C)c2)c(C)c1)OCCSC(C)=O] | 14 |
| 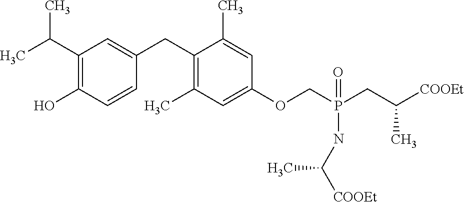 [SMILES: CCOC(=O)[C@H](C)CP(=O)(COc1cc(C)c(Cc2ccc(O)c(C(C)C)c2)c(C)c1)N[C@@H](C)C(=O)OCC] | 15-1 |
| 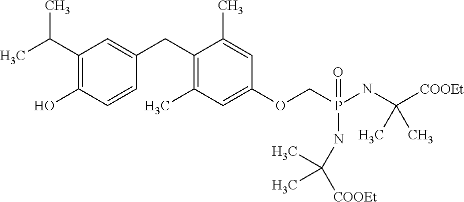 [SMILES: CCOC(=O)C(C)(C)NP(=O)(COc1cc(C)c(Cc2ccc(O)c(C(C)C)c2)c(C)c1)NC(C)(C)C(=O)OCC] | 15-2 |
| 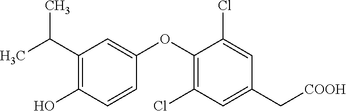 [SMILES: CC(C)c1cc(Oc2c(Cl)cc(CC(=O)O)cc2Cl)ccc1O] | 18 |
| 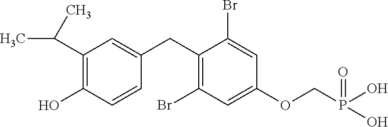 [SMILES: CC(C)c1cc(Cc2c(Br)cc(OCP(=O)(O)O)cc2Br)ccc1O] | 8-1 |
| 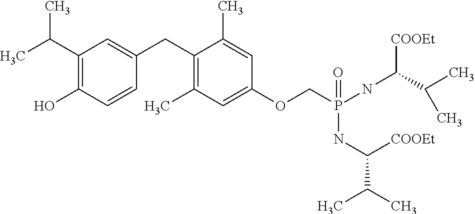 [SMILES: CCOC(=O)[C@@H](NP(=O)(COc1cc(C)c(Cc2ccc(O)c(C(C)C)c2)c(C)c1)N[C@H](C(=O)OCC)C(C)C)C(C)C] | 15-3 |
| 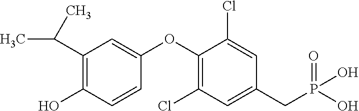 [SMILES: CC(C)c1cc(Oc2c(Cl)cc(CP(=O)(O)O)cc2Cl)ccc1O] | 19 |
| 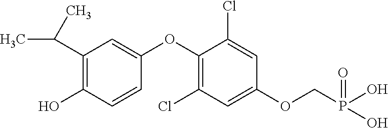 [SMILES: CC(C)c1cc(Oc2c(Cl)cc(OCP(=O)(O)O)cc2Cl)ccc1O] | 8-2 |
| 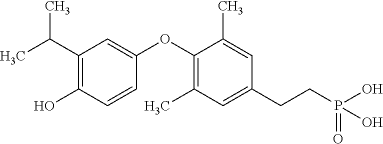 [SMILES: Cc1cc(CCP(=O)(O)O)cc(C)c1Oc1ccc(O)c(C(C)C)c1] | 24-1 |
| 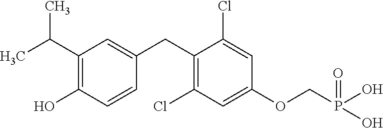 [SMILES: CC(C)c1cc(Cc2c(Cl)cc(OCP(=O)(O)O)cc2Cl)ccc1O] | 7-5 |
| 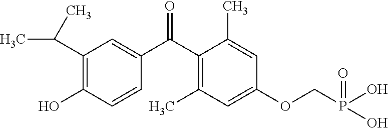 [SMILES: Cc1cc(OCP(=O)(O)O)cc(C)c1C(=O)c1ccc(O)c(C(C)C)c1] | 25 |
| 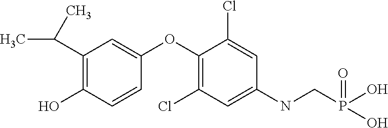 [SMILES: CC(C)c1cc(Oc2c(Cl)cc(NCP(=O)(O)O)cc2Cl)ccc1O] | 22 |
| 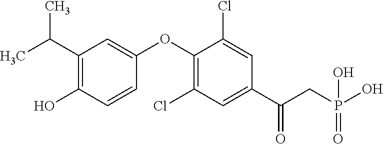 [SMILES: CC(C)c1cc(Oc2c(Cl)cc(C(=O)CP(=O)(O)O)cc2Cl)ccc1O] | 21 |
| 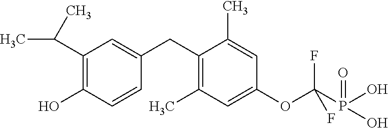 [SMILES: Cc1cc(OC(F)(F)P(=O)(O)O)cc(C)c1Cc1ccc(O)c(C(C)C)c1] | 7-6 |
| 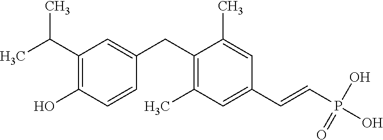 [SMILES: Cc1cc(/C=C/P(=O)(O)O)cc(C)c1Cc1ccc(O)c(C(C)C)c1] | 24-2 |
| 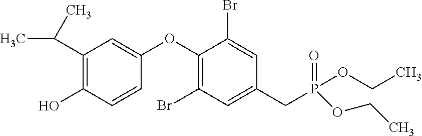 [SMILES: CCOP(=O)(Cc1cc(Br)c(Oc2ccc(O)c(C(C)C)c2)c(Br)c1)OCC] | 19-1 |
| 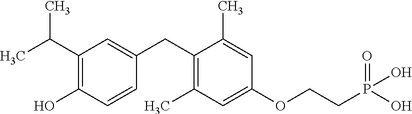 [SMILES: Cc1cc(OCCP(=O)(O)O)cc(C)c1Cc1ccc(O)c(C(C)C)c1] | 26 |
| 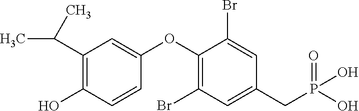 [SMILES: CC(C)c1cc(Oc2c(Br)cc(CP(=O)(O)O)cc2Br)ccc1O] | 19-2 |
| 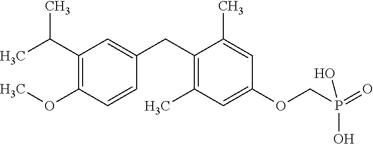 [SMILES: COc1ccc(Cc2c(C)cc(OCP(=O)(O)O)cc2C)cc1C(C)C] | 7-4 |
| 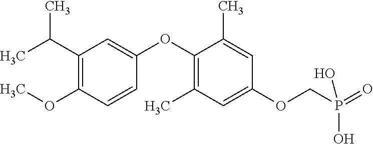 [SMILES: COc1ccc(Oc2c(C)cc(OCP(=O)(O)O)cc2C)cc1C(C)C] | 30 |
| 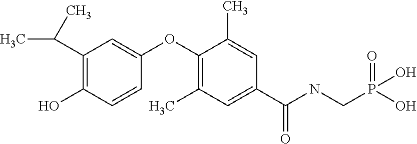 [SMILES: Cc1cc(C(=O)NCP(=O)(O)O)cc(C)c1Oc1ccc(O)c(C(C)C)c1] | 23 |
| 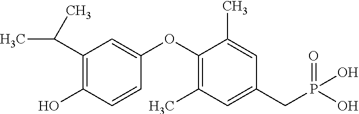 [SMILES: Cc1cc(CP(=O)(O)O)cc(C)c1Oc1ccc(O)c(C(C)C)c1] | 19-3 |
| 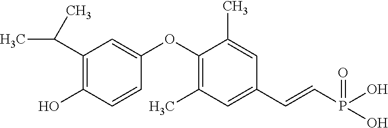 [SMILES: Cc1cc(/C=C/P(=O)(O)O)cc(C)c1Oc1ccc(O)c(C(C)C)c1] | 28 |
| 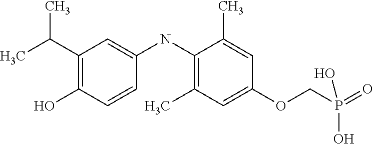 [SMILES: Cc1cc(OCP(=O)(O)O)cc(C)c1Nc1ccc(O)c(C(C)C)c1] | 20 |
| 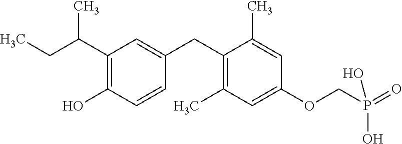 [SMILES: CCC(C)c1cc(Cc2c(C)cc(OCP(=O)(O)O)cc2C)ccc1O] | 7-3 |
| 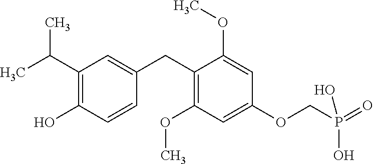 [SMILES: COc1cc(OCP(=O)(O)O)cc(OC)c1Cc1ccc(O)c(C(C)C)c1] | 7-2 |
| 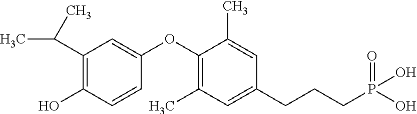 [SMILES: Cc1cc(CCCP(=O)(O)O)cc(C)c1Oc1ccc(O)c(C(C)C)c1] | 29 |
| 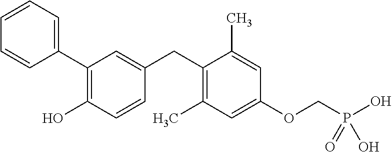 [SMILES: Cc1cc(OCP(=O)(O)O)cc(C)c1Cc1ccc(O)c(-c2ccccc2)c1] | 7-1 |
| 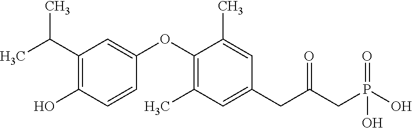 [SMILES: Cc1cc(CC(=O)CP(=O)(O)O)cc(C)c1Oc1ccc(O)c(C(C)C)c1] | 32 |
| 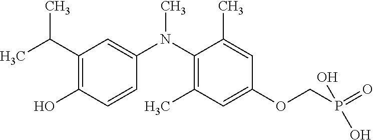 [SMILES: Cc1cc(OCP(=O)(O)O)cc(C)c1N(C)c1ccc(O)c(C(C)C)c1] | 20-1 |
| 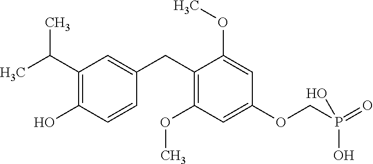 [SMILES: COc1cc(OCP(=O)(O)O)cc(OC)c1Cc1ccc(O)c(C(C)C)c1] | 24 |
| 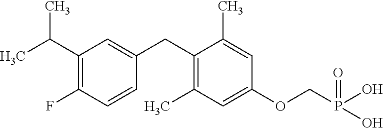 [SMILES: Cc1cc(OCP(=O)(O)O)cc(C)c1Cc1ccc(F)c(C(C)C)c1] | 27 |
| 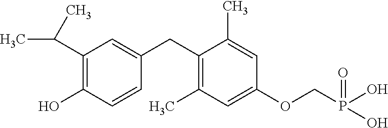 [SMILES: Cc1cc(OCP(=O)(O)O)cc(C)c1Cc1ccc(O)c(C(C)C)c1] | 31 |
| 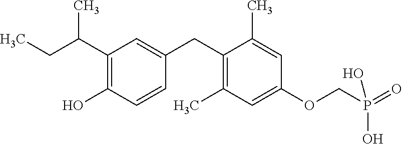 [SMILES: CCC(C)c1cc(Cc2c(C)cc(OCP(=O)(O)O)cc2C)ccc1O] | 24-3 |
| 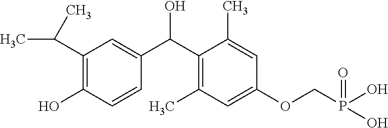 [SMILES: Cc1cc(OCP(=O)(O)O)cc(C)c1C(O)c1ccc(O)c(C(C)C)c1] | 33 |
| 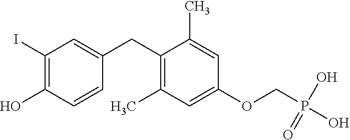 [SMILES: Cc1cc(OCP(=O)(O)O)cc(C)c1Cc1ccc(O)c(I)c1] | 34 |
| 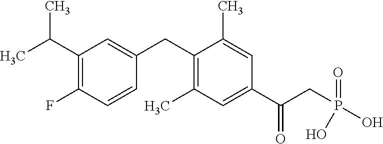 [SMILES: Cc1cc(C(=O)CP(=O)(O)O)cc(C)c1Cc1ccc(F)c(C(C)C)c1] | 41-2 |
| 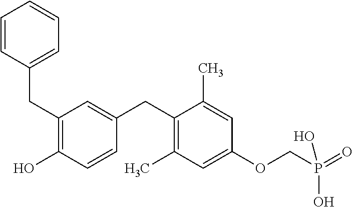 [SMILES: Cc1cc(OCP(=O)(O)O)cc(C)c1Cc1ccc(O)c(Cc2ccccc2)c1] | 38 |
| 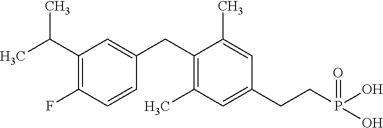 [SMILES: Cc1cc(CCP(=O)(O)O)cc(C)c1Cc1ccc(F)c(C(C)C)c1] | 42-2 |
| 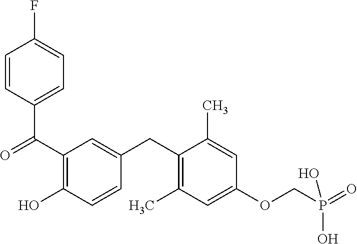 [SMILES: Cc1cc(OCP(=O)(O)O)cc(C)c1Cc1ccc(O)c(C(=O)c2ccc(F)cc2)c1] | 39 |
| 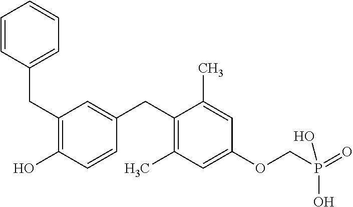 [SMILES: Cc1cc(OCP(=O)(O)O)cc(C)c1Cc1ccc(O)c(Cc2ccccc2)c1] | 41 |
| 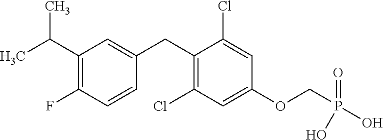 [SMILES: CC(C)c1cc(Cc2c(Cl)cc(OCP(=O)(O)O)cc2Cl)ccc1F] | 27-2 |
| 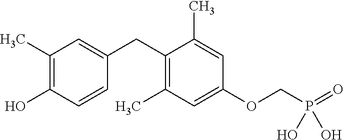 [SMILES: Cc1cc(Cc2c(C)cc(OCP(=O)(O)O)cc2C)ccc1O] | 7-7 |
| 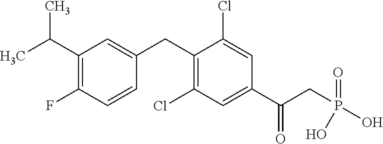 [SMILES: CC(C)c1cc(Cc2c(Cl)cc(C(=O)CP(=O)(O)O)cc2Cl)ccc1F] | 41-3 |
| 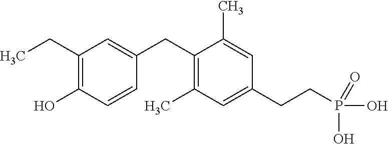 [SMILES: CCc1cc(Cc2c(C)cc(CCP(=O)(O)O)cc2C)ccc1O] | 24-4 |
| 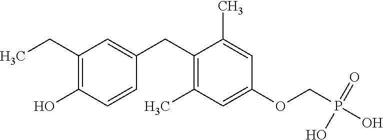 [SMILES: CCc1cc(Cc2c(C)cc(OCP(=O)(O)O)cc2C)ccc1O] | 7-8 |
| 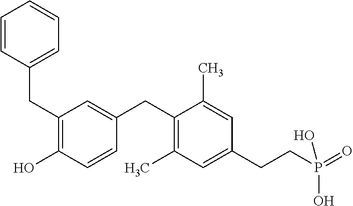 [SMILES: Cc1cc(CCP(=O)(O)O)cc(C)c1Cc1ccc(O)c(Cc2ccccc2)c1] | 42 |
| 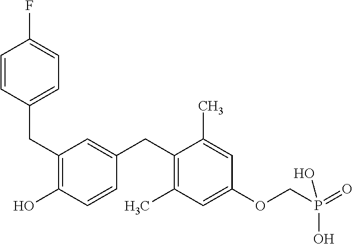 [SMILES: Cc1cc(OCP(=O)(O)O)cc(C)c1Cc1ccc(O)c(Cc2ccc(F)cc2)c1] | 40 |
| 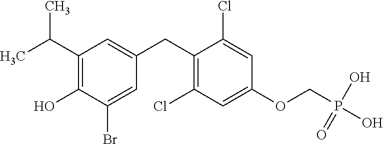 [SMILES: CC(C)c1cc(Cc2c(Cl)cc(OCP(=O)(O)O)cc2Cl)cc(Br)c1O] | 7-14 |
| 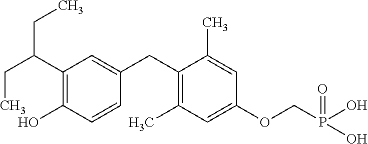 [SMILES: CCC(CC)c1cc(Cc2c(C)cc(OCP(=O)(O)O)cc2C)ccc1O] | 7-9 |
| 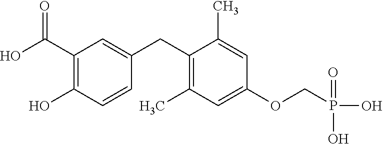 [SMILES: Cc1cc(OCP(=O)(O)O)cc(C)c1Cc1ccc(O)c(C(=O)O)c1] | 35 |
| 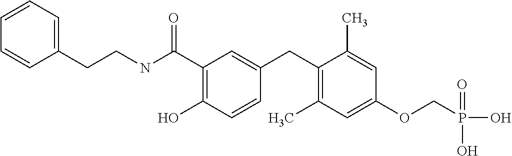 [SMILES: Cc1cc(OCP(=O)(O)O)cc(C)c1Cc1ccc(O)c(C(=O)NCCc2ccccc2)c1] | 37 |
| 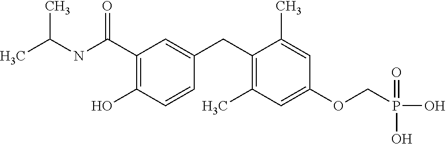 [SMILES: Cc1cc(OCP(=O)(O)O)cc(C)c1Cc1ccc(O)c(C(=O)NC(C)C)c1] | 36 |
| 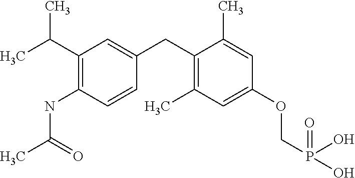 [SMILES: CC(=O)Nc1ccc(Cc2c(C)cc(OCP(=O)(O)O)cc2C)cc1C(C)C] | 7-12 |
| 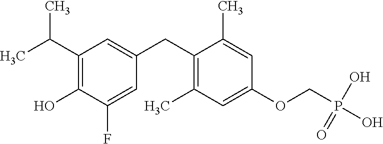 [SMILES: Cc1cc(OCP(=O)(O)O)cc(C)c1Cc1cc(F)c(O)c(C(C)C)c1] | 7-11 |
| 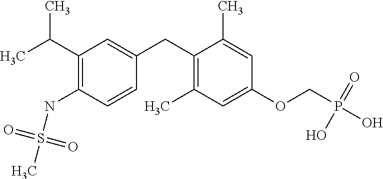 [SMILES: Cc1cc(OCP(=O)(O)O)cc(C)c1Cc1ccc(NS(C)(=O)=O)c(C(C)C)c1] | 7-13 |
| 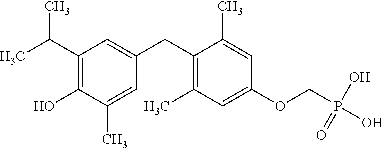 [SMILES: Cc1cc(Cc2c(C)cc(OCP(=O)(O)O)cc2C)cc(C(C)C)c1O] | 7-10 |
| 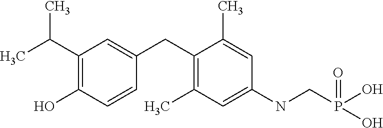 [SMILES: Cc1cc(NCP(=O)(O)O)cc(C)c1Cc1ccc(O)c(C(C)C)c1] | 47 |
| 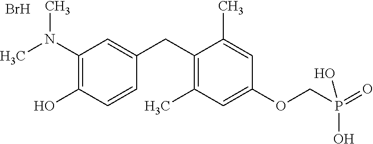 [SMILES: Br ; Cc1cc(OCP(=O)(O)O)cc(C)c1Cc1ccc(O)c(N(C)C)c1] | 49 |
| 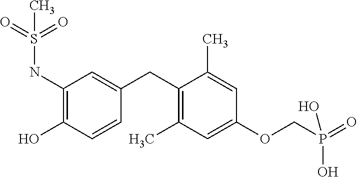 [SMILES: Cc1cc(OCP(=O)(O)O)cc(C)c1Cc1ccc(O)c(NS(C)(=O)=O)c1] | 51-1 |
| 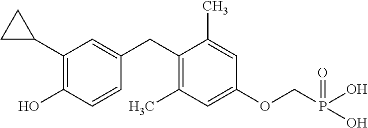 [SMILES: Cc1cc(OCP(=O)(O)O)cc(C)c1Cc1ccc(O)c(C2CC2)c1] | 48 |
| 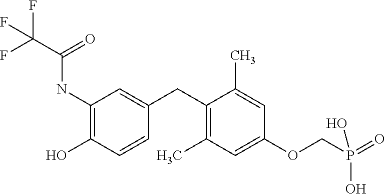 [SMILES: Cc1cc(OCP(=O)(O)O)cc(C)c1Cc1ccc(O)c(NC(=O)C(F)(F)F)c1] | 51-2 |
| 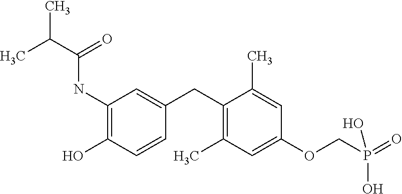 [SMILES: Cc1cc(OCP(=O)(O)O)cc(C)c1Cc1ccc(O)c(NC(=O)C(C)C)c1] | 51-3 |
| 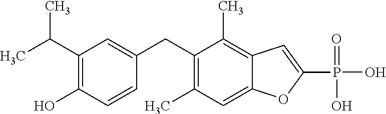 [SMILES: Cc1cc2oc(P(=O)(O)O)cc2c(C)c1Cc1ccc(O)c(C(C)C)c1] | 45 |
| 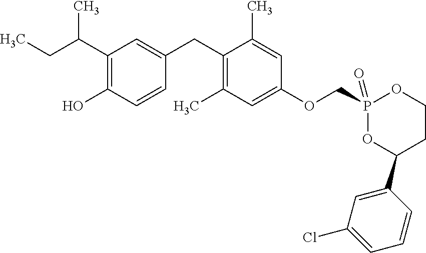 [SMILES: CCC(C)c1cc(Cc2c(C)cc(OC[P@@]3(=O)OCC[C@@H](c4cccc(Cl)c4)O3)cc2C)ccc1O] | 13-8 |
| 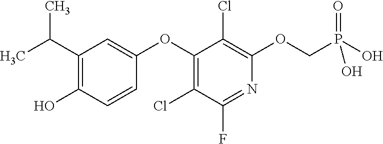 [SMILES: CC(C)c1cc(Oc2c(Cl)c(F)nc(OCP(=O)(O)O)c2Cl)ccc1O] | 57 |
| 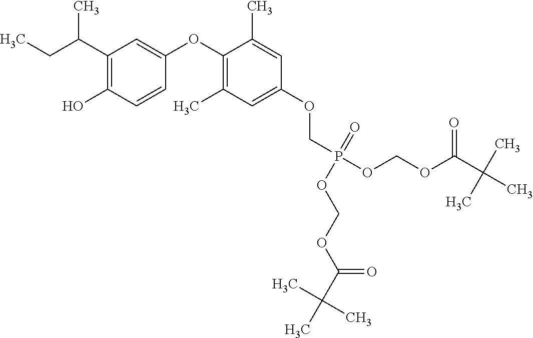 [SMILES: CCC(C)c1cc(Oc2c(C)cc(OCP(=O)(OCOC(=O)C(C)(C)C)OCOC(=O)C(C)(C)C)cc2C)ccc1O] | 12-4 |
| 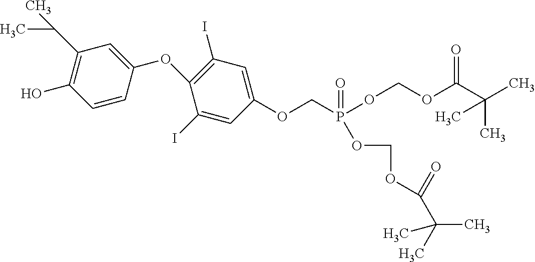 [SMILES: CC(C)c1cc(Oc2c(I)cc(OCP(=O)(OCOC(=O)C(C)(C)C)OCOC(=O)C(C)(C)C)cc2I)ccc1O] | 12-7 |
| 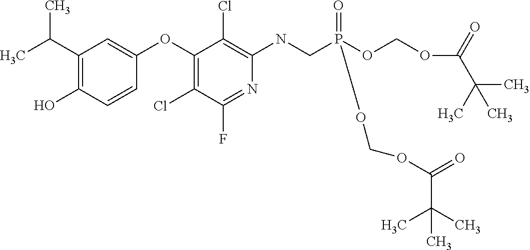 [SMILES: CC(C)c1cc(Oc2c(Cl)c(F)nc(NCP(=O)(OCOC(=O)C(C)(C)C)OCOC(=O)C(C)(C)C)c2Cl)ccc1O] | 12-9 |
| 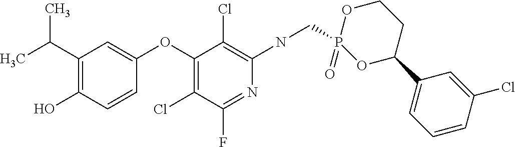 [SMILES: CC(C)c1cc(Oc2c(Cl)c(F)nc(NC[P@]3(=O)OCC[C@@H](c4cccc(Cl)c4)O3)c2Cl)ccc1O] | 13-12-trans |
| 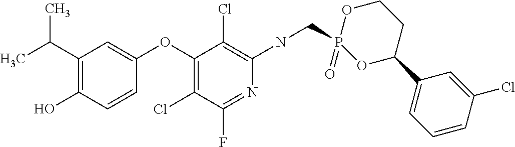 [SMILES: CC(C)c1cc(Oc2c(Cl)c(F)nc(NC[P@@]3(=O)OCC[C@@H](c4cccc(Cl)c4)O3)c2Cl)ccc1O] | 13-12-cis |
| 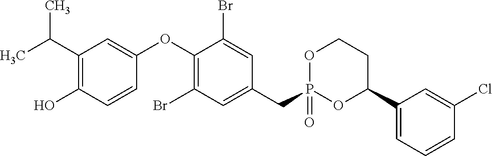 [SMILES: CC(C)c1cc(Oc2c(Br)cc(C[P@@]3(=O)OCC[C@@H](c4cccc(Cl)c4)O3)cc2Br)ccc1O] | 13-9 |
| 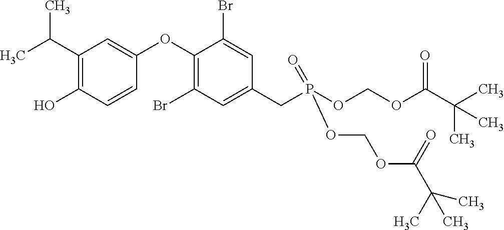 [SMILES: CC(C)c1cc(Oc2c(Br)cc(CP(=O)(OCOC(=O)C(C)(C)C)OCOC(=O)C(C)(C)C)cc2Br)ccc1O] | 12-5 |
| 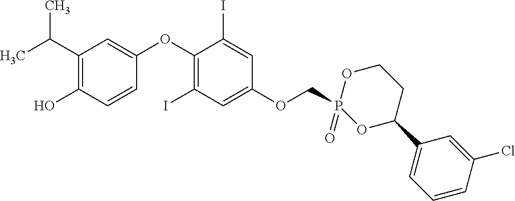 [SMILES: CC(C)c1cc(Oc2c(I)cc(OC[P@@]3(=O)OCC[C@@H](c4cccc(Cl)c4)O3)cc2I)ccc1O] | 13-10 |
| 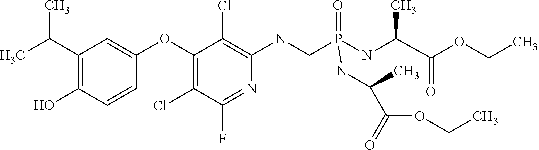 [SMILES: CCOC(=O)[C@H](C)NP(=O)(CNc1nc(F)c(Cl)c(Oc2ccc(O)c(C(C)C)c2)c1Cl)N[C@@H](C)C(=O)OCC] | 15-6 |
| 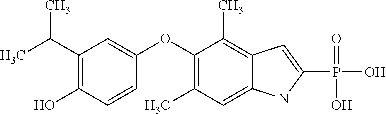 [SMILES: Cc1cc2[nH]c(P(=O)(O)O)cc2c(C)c1Oc1ccc(O)c(C(C)C)c1] | 66 |
| 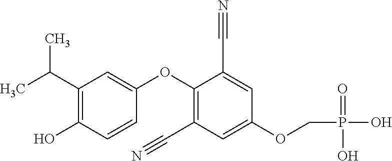 [SMILES: CC(C)c1cc(Oc2c(C#N)cc(OCP(=O)(O)O)cc2C#N)ccc1O] | 56 |
| 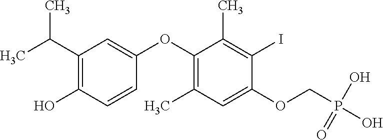 [SMILES: Cc1cc(OCP(=O)(O)O)c(I)c(C)c1Oc1ccc(O)c(C(C)C)c1] | 46 |
| 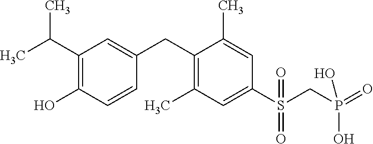 [SMILES: Cc1cc(S(=O)(=O)CP(=O)(O)O)cc(C)c1Cc1ccc(O)c(C(C)C)c1] | 52 |
| 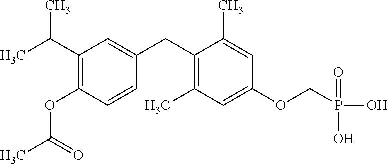 [SMILES: CC(=O)Oc1ccc(Cc2c(C)cc(OCP(=O)(O)O)cc2C)cc1C(C)C] | 58 |
| 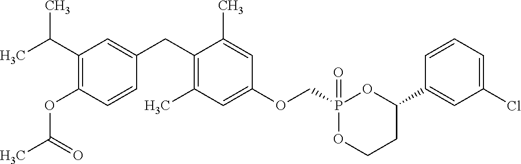 [SMILES: CC(=O)Oc1ccc(Cc2c(C)cc(OC[P@@]3(=O)OCC[C@@H](c4cccc(Cl)c4)O3)cc2C)cc1C(C)C] | 59 |
| 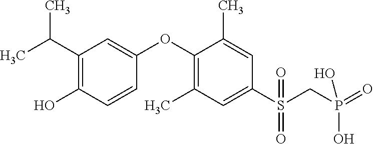 [SMILES: Cc1cc(S(=O)(=O)CP(=O)(O)O)cc(C)c1Oc1ccc(O)c(C(C)C)c1] | 53 |
| 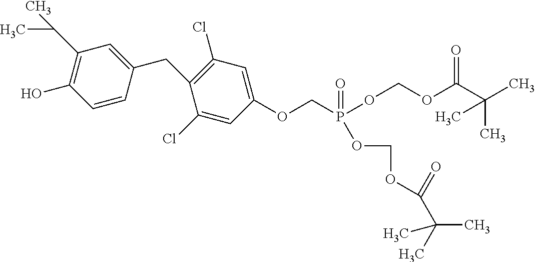 [SMILES: CC(C)c1cc(Cc2c(Cl)cc(OCP(=O)(OCOC(=O)C(C)(C)C)OCOC(=O)C(C)(C)C)cc2Cl)ccc1O] | 12-8 |
| 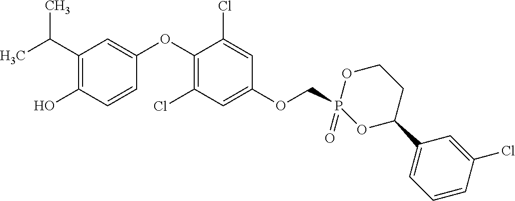 [SMILES: CC(C)c1cc(Oc2c(Cl)cc(OC[P@@]3(=O)OCC[C@@H](c4cccc(Cl)c4)O3)cc2Cl)ccc1O] | 13-11 |
| 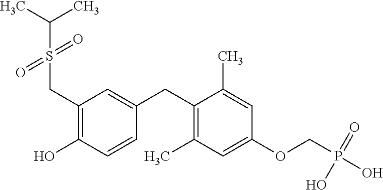 [SMILES: Cc1cc(OCP(=O)(O)O)cc(C)c1Cc1ccc(O)c(CS(=O)(=O)C(C)C)c1] | 44 |
| 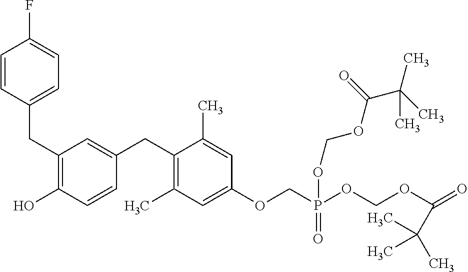 [SMILES: Cc1cc(OCP(=O)(OCOC(=O)C(C)(C)C)OCOC(=O)C(C)(C)C)cc(C)c1Cc1ccc(O)c(Cc2ccc(F)cc2)c1] | 12-6 |
| 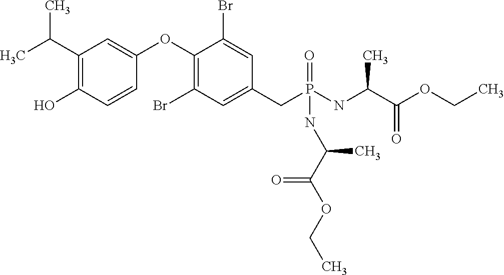 [SMILES: CCOC(=O)[C@H](C)NP(=O)(Cc1cc(Br)c(Oc2ccc(O)c(C(C)C)c2)c(Br)c1)N[C@@H](C)C(=O)OCC] | 15-5 |
| 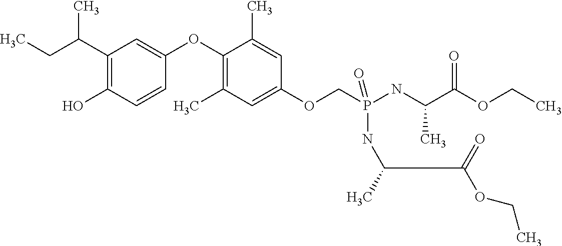 [SMILES: CCOC(=O)[C@H](C)NP(=O)(COc1cc(C)c(Oc2ccc(O)c(C(C)CC)c2)c(C)c1)N[C@@H](C)C(=O)OCC] | 15-4 |
| 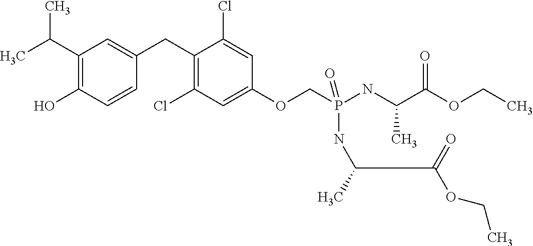 [SMILES: CCOC(=O)[C@H](C)NP(=O)(COc1cc(Cl)c(Cc2ccc(O)c(C(C)C)c2)c(Cl)c1)N[C@@H](C)C(=O)OCC] | 15-7 |
| 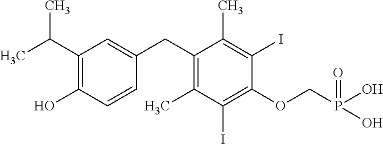 [SMILES: Cc1c(I)c(OCP(=O)(O)O)c(I)c(C)c1Cc1ccc(O)c(C(C)C)c1] | 65-1 |
| 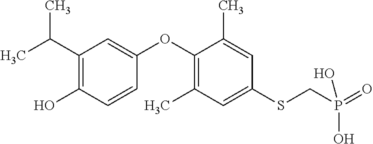 [SMILES: Cc1cc(SCP(=O)(O)O)cc(C)c1Oc1ccc(O)c(C(C)C)c1] | 54 |
| 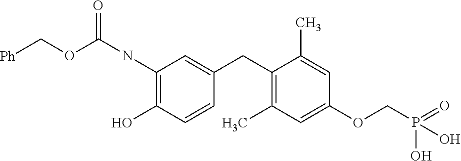 [SMILES: Cc1cc(OCP(=O)(O)O)cc(C)c1Cc1ccc(O)c(NC(=O)OCc2ccccc2)c1] | 50 |
| 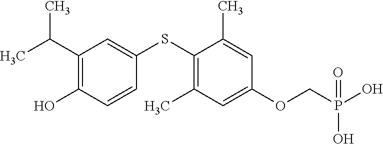 [SMILES: Cc1cc(OCP(=O)(O)O)cc(C)c1Sc1ccc(O)c(C(C)C)c1] | 43 |
| 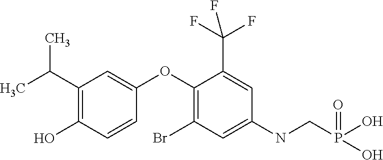 [SMILES: CC(C)c1cc(Oc2c(Br)cc(NCP(=O)(O)O)cc2C(F)(F)F)ccc1O] | 63 |
| 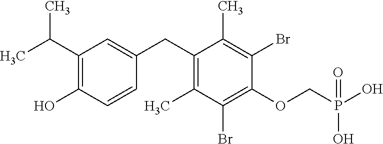 [SMILES: Cc1c(Br)c(OCP(=O)(O)O)c(Br)c(C)c1Cc1ccc(O)c(C(C)C)c1] | 65-2 |
| 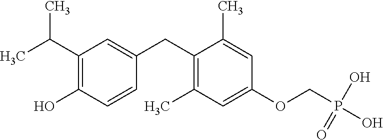 [SMILES: Cc1cc(OCP(=O)(O)O)cc(C)c1Cc1ccc(O)c(C(C)C)c1] | 7-16 |
| 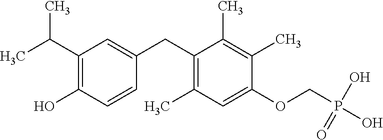 [SMILES: Cc1cc(OCP(=O)(O)O)c(C)c(C)c1Cc1ccc(O)c(C(C)C)c1] | 61 |
| 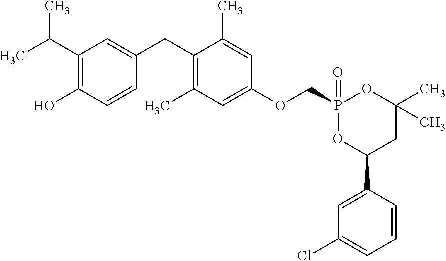 [SMILES: Cc1cc(OC[P@]2(=O)O[C@H](c3cccc(Cl)c3)CC(C)(C)O2)cc(C)c1Cc1ccc(O)c(C(C)C)c1] | 13-13-cis |
| 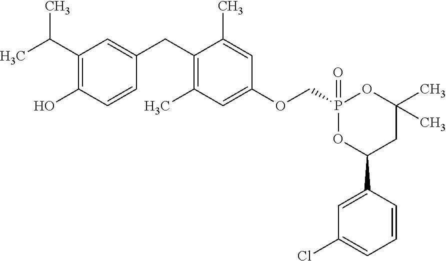 [SMILES: Cc1cc(OC[P@@]2(=O)O[C@H](c3cccc(Cl)c3)CC(C)(C)O2)cc(C)c1Cc1ccc(O)c(C(C)C)c1] | 13-13-trans |
| 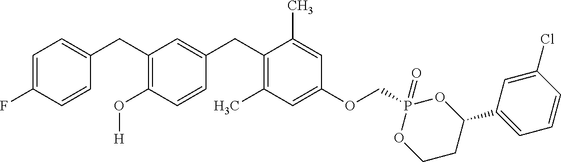 [SMILES: Cc1cc(OC[P@@]2(=O)OCC[C@@H](c3cccc(Cl)c3)O2)cc(C)c1Cc1ccc(O)c(Cc2ccc(F)cc2)c1] | 13-14-cis |
| 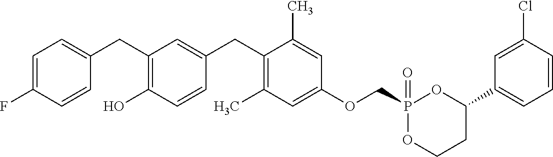 [SMILES: Cc1cc(OC[P@]2(=O)OCC[C@@H](c3cccc(Cl)c3)O2)cc(C)c1Cc1ccc(O)c(Cc2ccc(F)cc2)c1] | 13-14-trans |
| 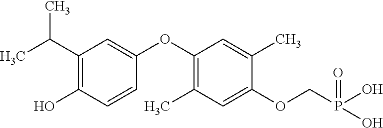 [SMILES: Cc1cc(Oc2ccc(O)c(C(C)C)c2)c(C)cc1OCP(=O)(O)O] | 7-17 |
| 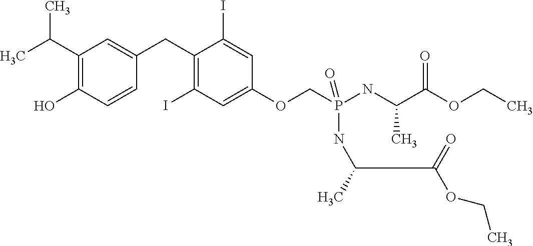 [SMILES: CCOC(=O)[C@H](C)NP(=O)(COc1cc(I)c(Cc2ccc(O)c(C(C)C)c2)c(I)c1)N[C@@H](C)C(=O)OCC] | 15-8 |
| 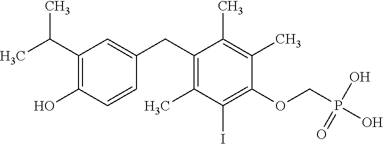 [SMILES: Cc1c(C)c(OCP(=O)(O)O)c(I)c(C)c1Cc1ccc(O)c(C(C)C)c1] | 62 |
| 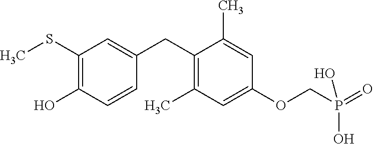 [SMILES: CSc1cc(Cc2c(C)cc(OCP(=O)(O)O)cc2C)ccc1O] | 55 |
| 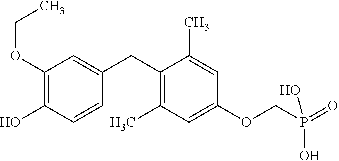 [SMILES: CCOc1cc(Cc2c(C)cc(OCP(=O)(O)O)cc2C)ccc1O] | 7-15 |

Preferred compositions for administration according to the methods of the present disclosure include those comprising Compounds 1, 2, 3 and/or 4, corresponding to the structures

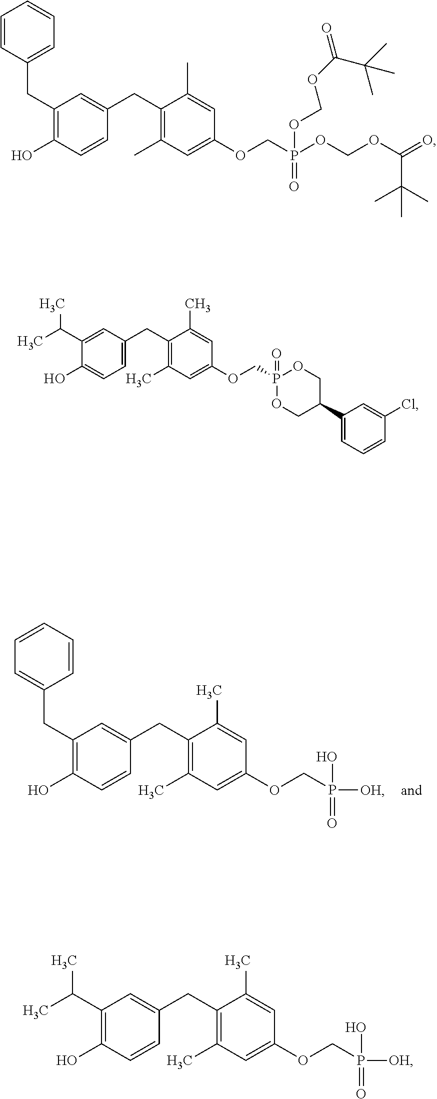

respectively, or pharmaceutically acceptable salts thereof.

In some embodiments, a compound to be administered according to the compositions and methods of the present disclosure may comprise one or more of the following:

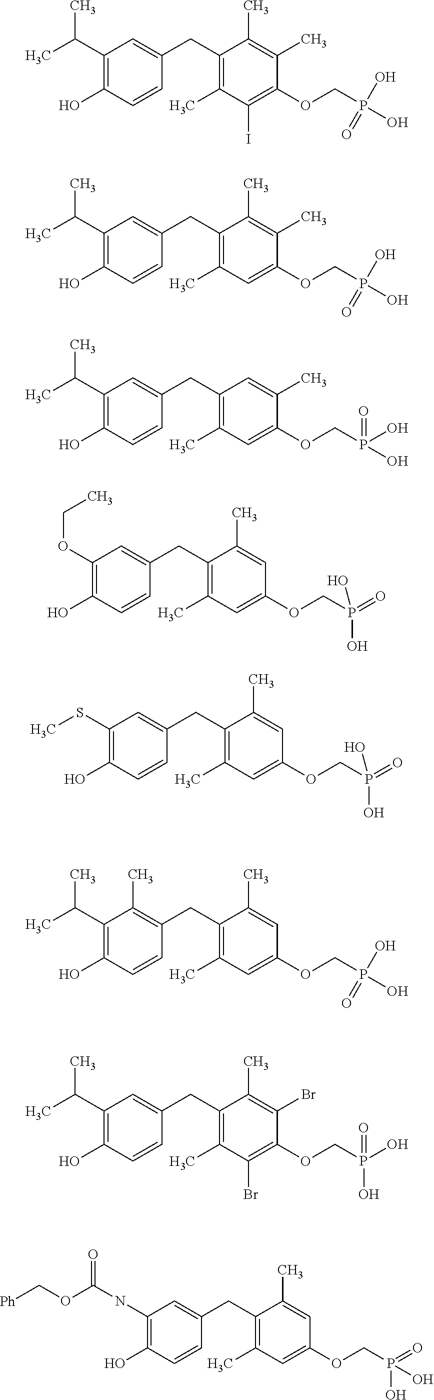

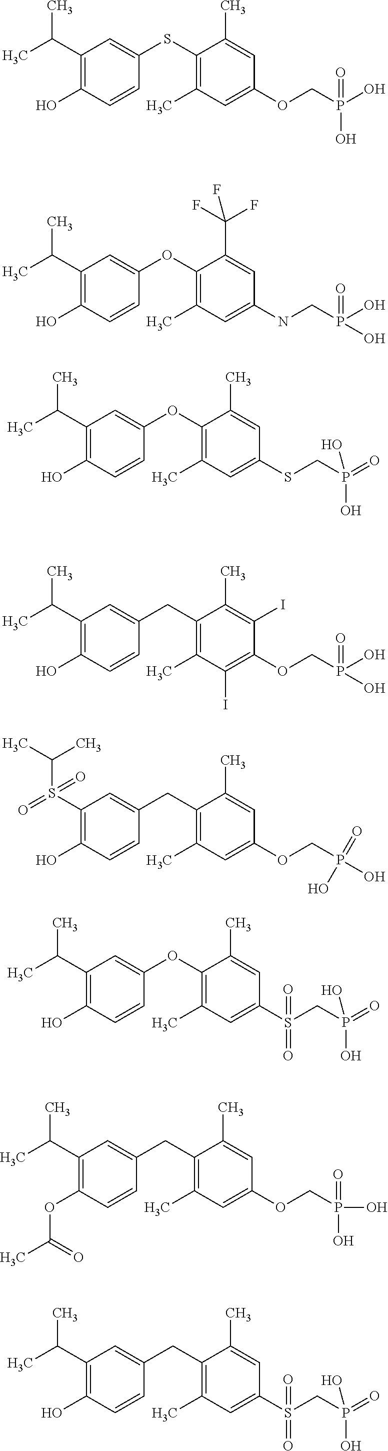

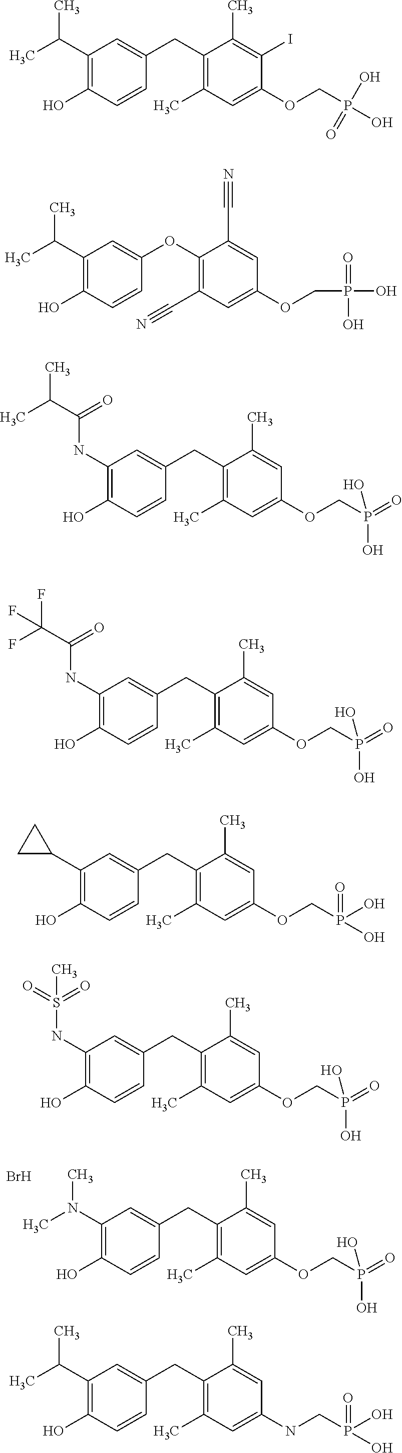

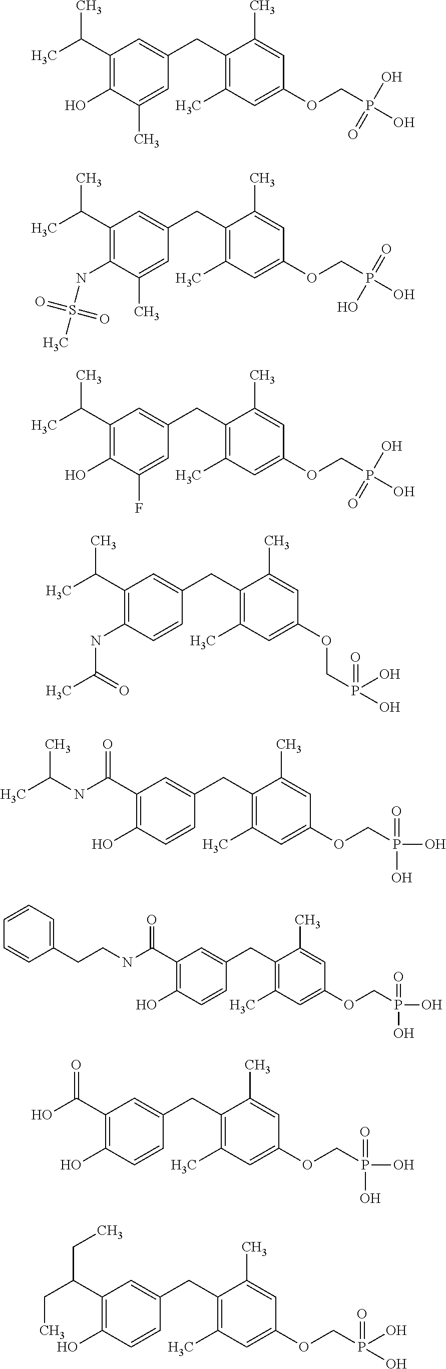

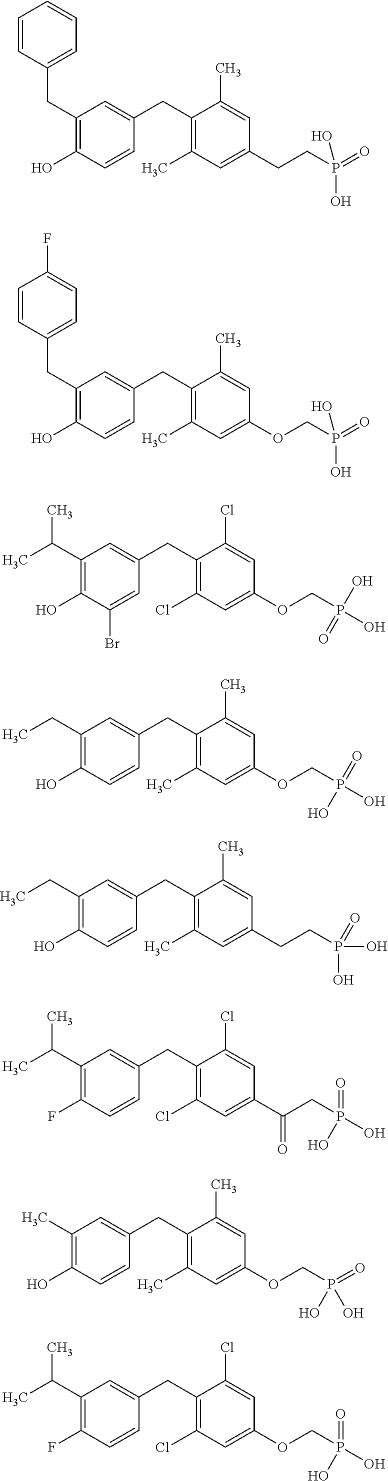

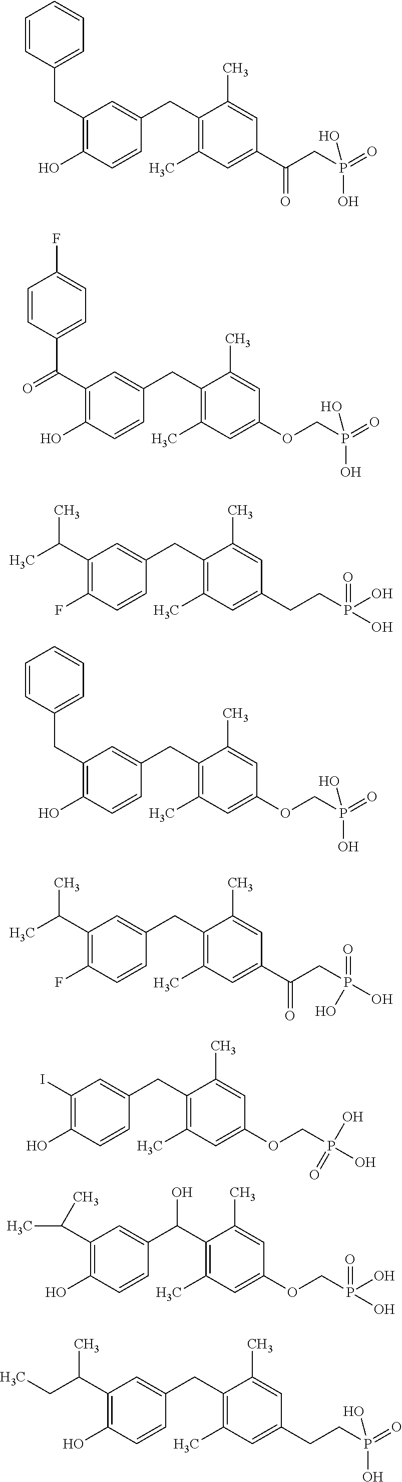

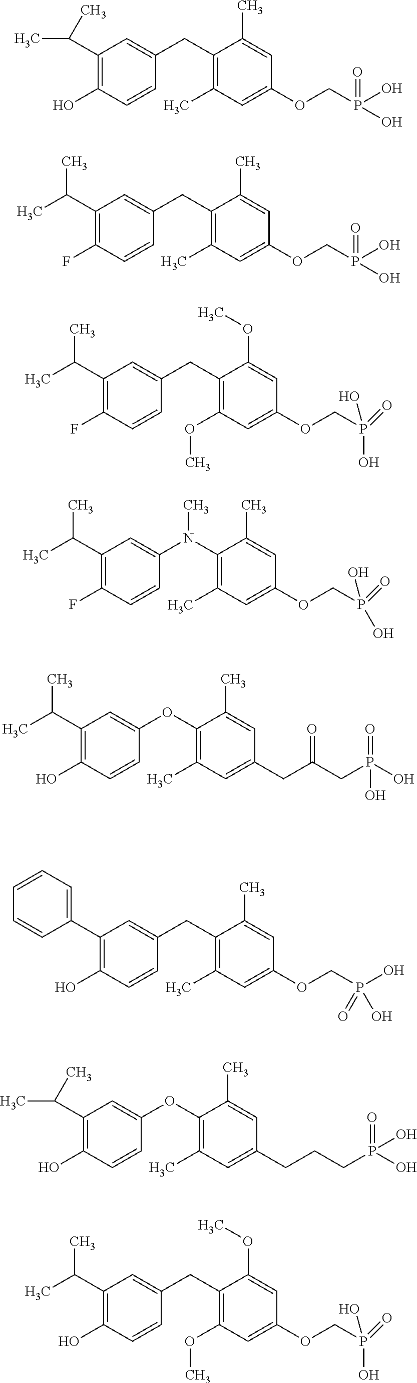

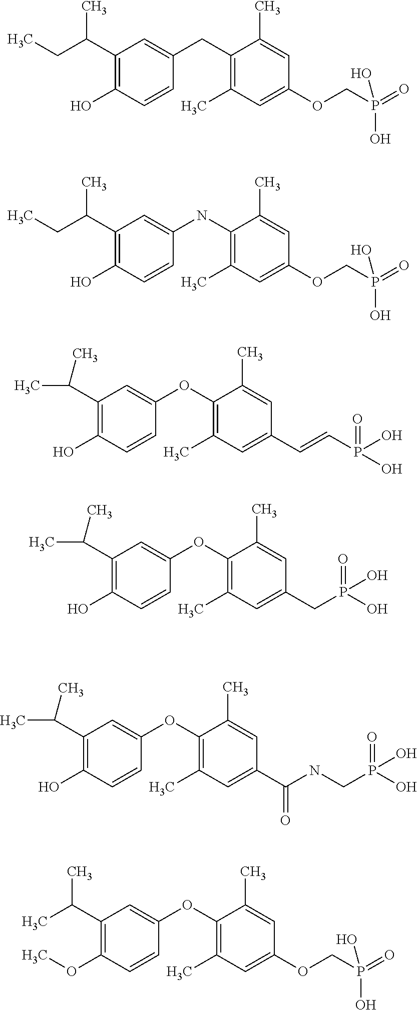 [SMILES: CCC(C)c1cc(Cc2c(C)cc(OCP(=O)(O)O)cc2C)ccc1O ; CCC(C)c1cc(Nc2c(C)cc(OCP(=O)(O)O)cc2C)ccc1O ; Cc1cc(/C=C/P(=O)(O)O)cc(C)c1Oc1ccc(O)c(C(C)C)c1 ; Cc1cc(CP(=O)(O)O)cc(C)c1Oc1ccc(O)c(C(C)C)c1 ; Cc1cc(C(=O)NCP(=O)(O)O)cc(C)c1Oc1ccc(O)c(C(C)C)c1 ; COc1ccc(Oc2c(C)cc(OCP(=O)(O)O)cc2C)cc1C(C)C]

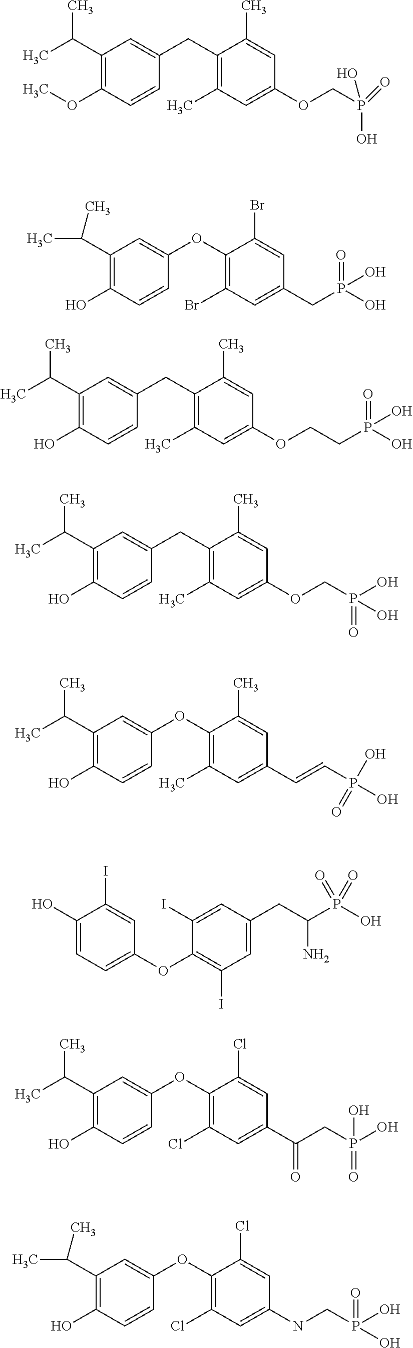

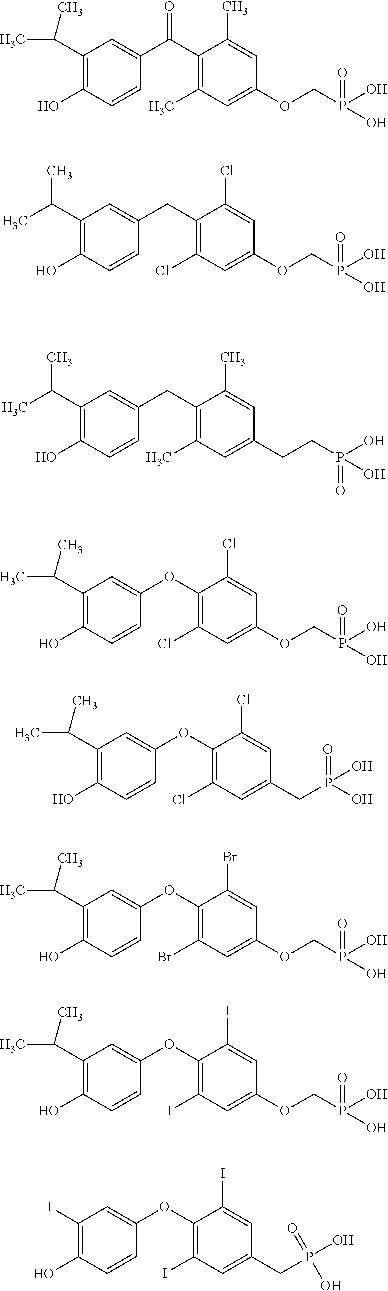

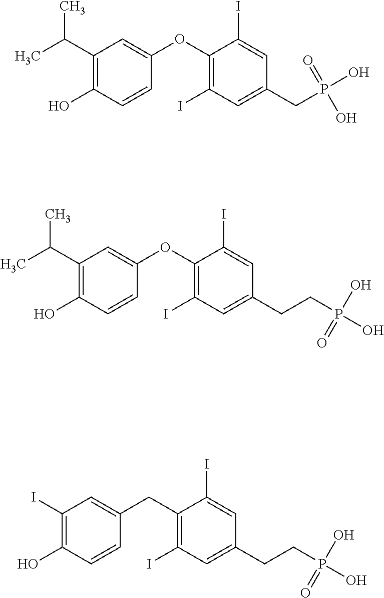 [SMILES: CC(C)c1cc(Oc2c(I)cc(CP(=O)(O)O)cc2I)ccc1O ; CC(C)c1cc(Oc2c(I)cc(CCP(=O)(O)O)cc2I)ccc1O ; O=P(O)(O)CCc1cc(I)c(Cc2ccc(O)c(I)c2)c(I)c1]

or pharmaceutically acceptable salts thereof, or any combination thereof.

In some other embodiments, a compound to be administered according to the compositions and methods of the present disclosure may comprise one or more of the following or pharmaceutically acceptable salts thereof:

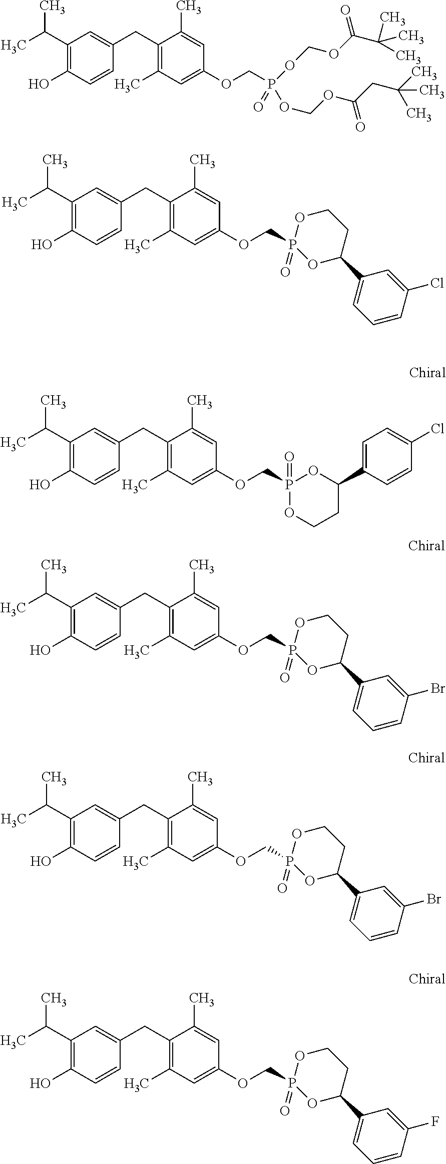

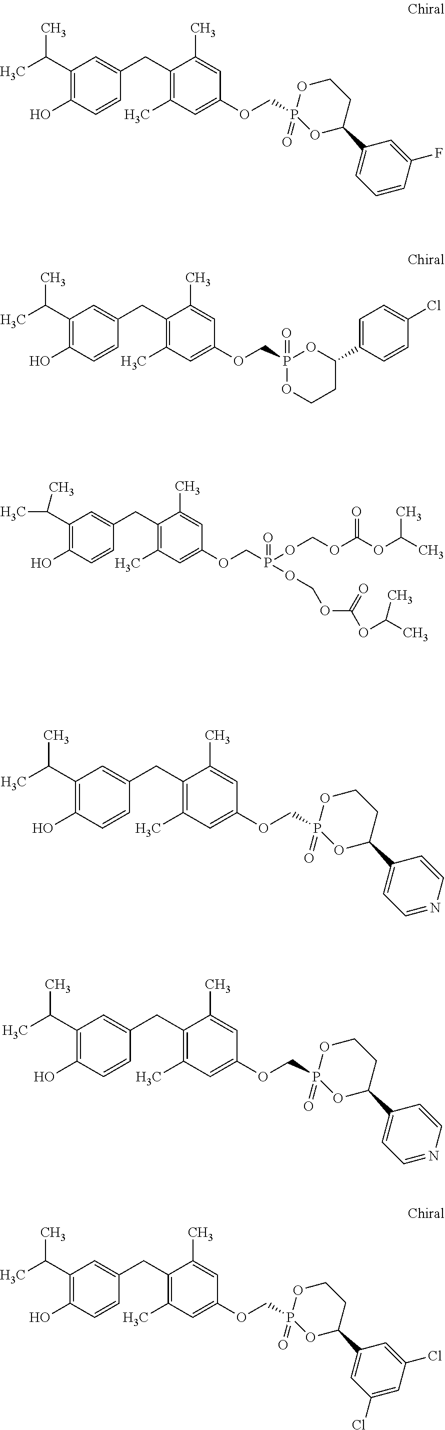

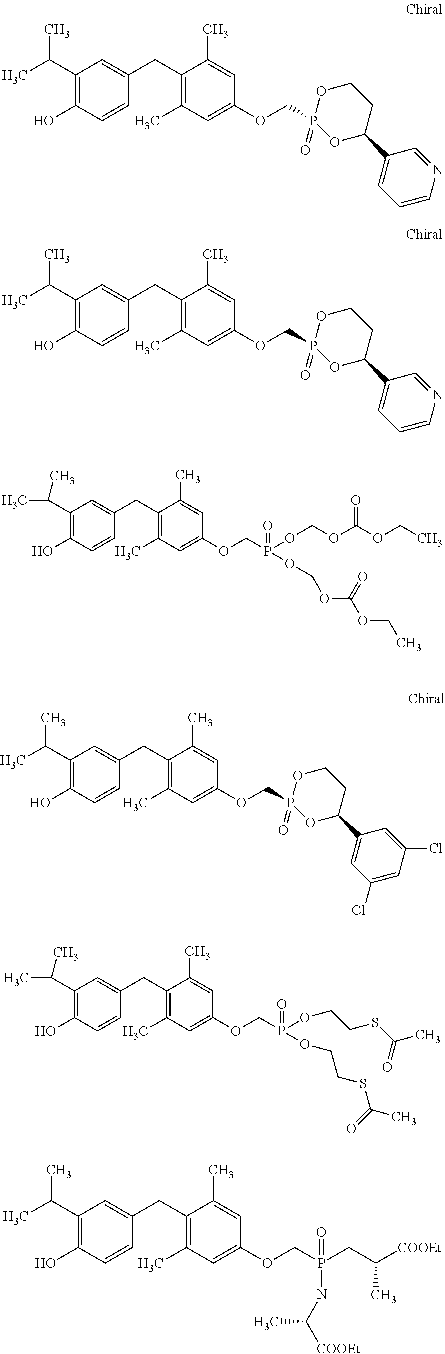

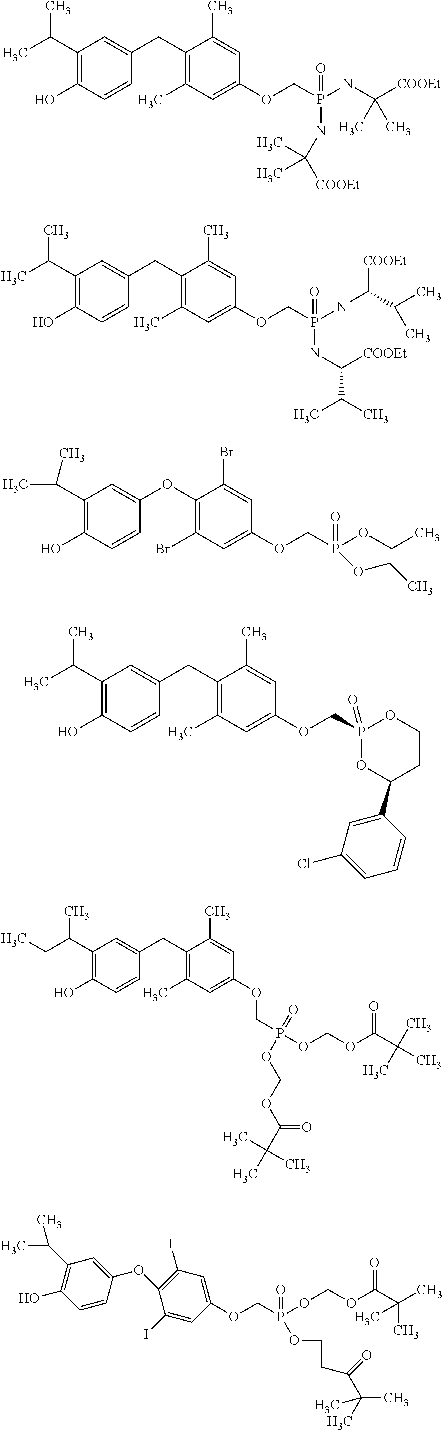

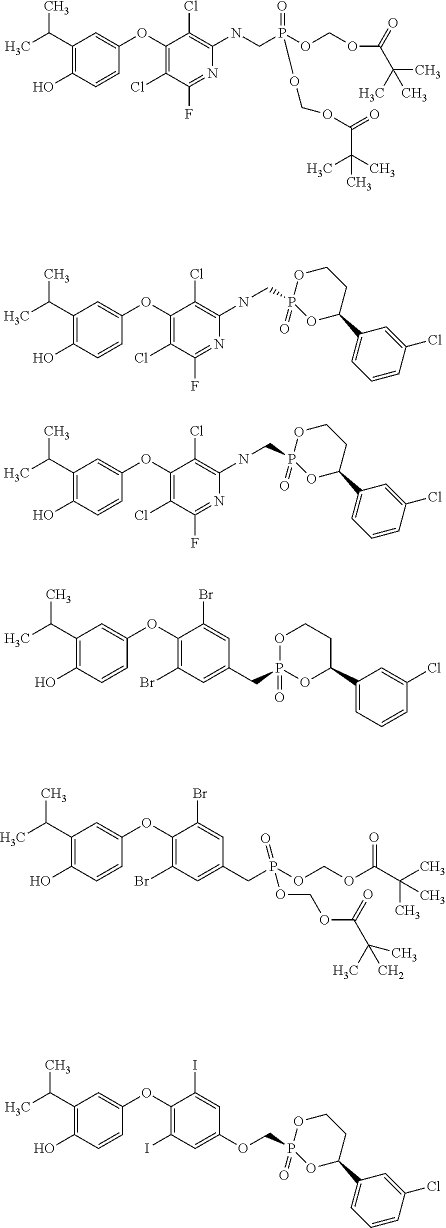

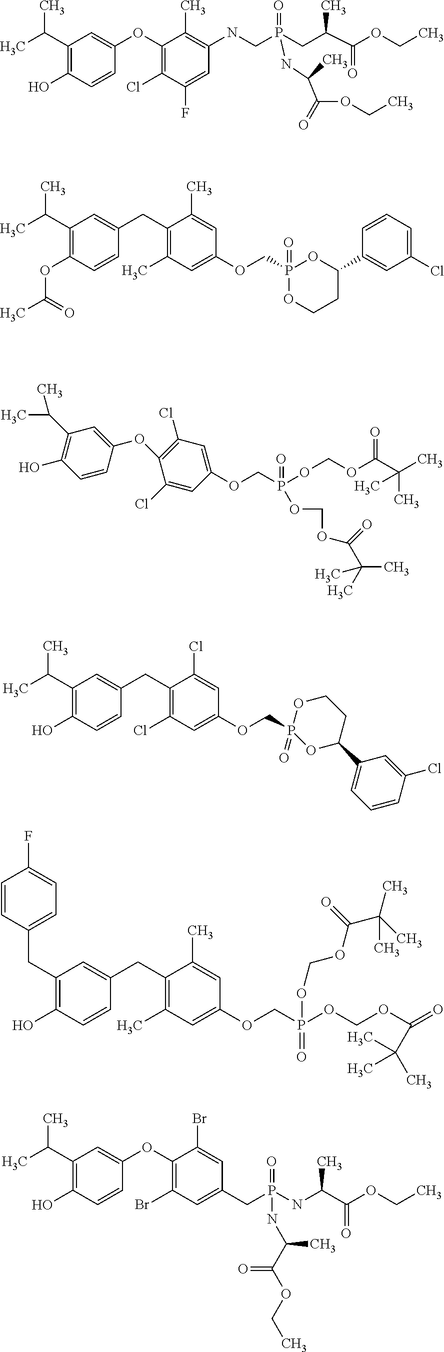

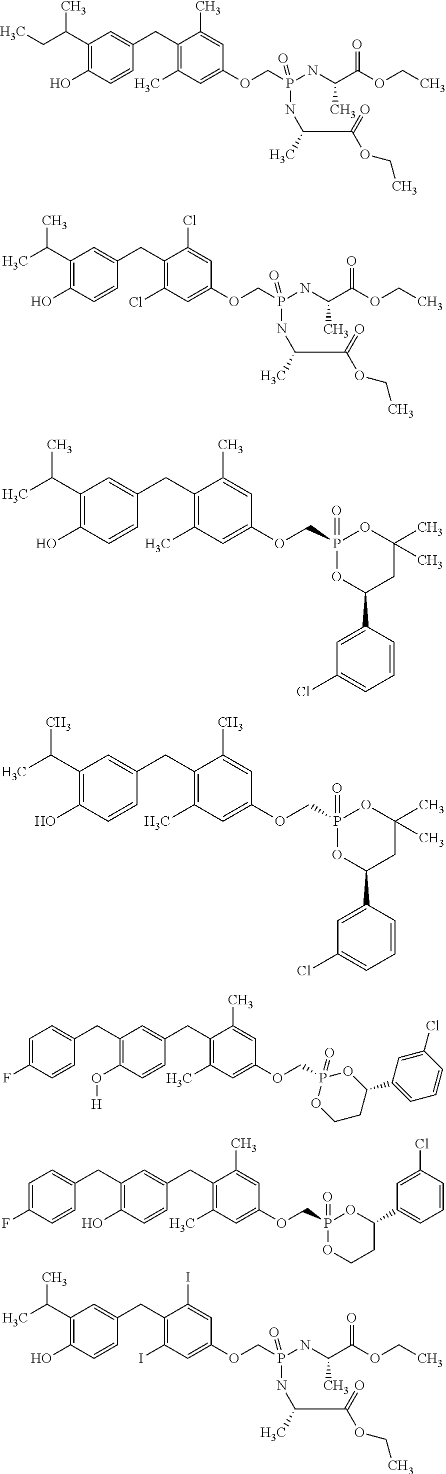

or any combination thereof.

In previous trials, daily administration of these compounds in dogs had led to both a dose-dependent decrease in serum lipid levels and a suppression of the HPT axis whose dose-dependence was unclear. Surprisingly, given the dose dependence of the primary lipid-lowering effect, changing the dosing schedule to every other day did not lead to a reduction in efficacy with respect to reducing plasma cholesterol, but did relieve the suppression of the HPT axis. Therefore, some embodiments include methods of administering Compound 1, Compound 2, and related compounds in such a way as to retain their primary effect as agonists of TRPβ, and the relief of clinical symptoms achieved thereby, while ameliorating or eliminating the suppression of the HPT axis and the concomitant side effects of such suppression.

According to the methods disclosed herein, reduction in HPT-suppression side effects related to administration of the above-mentioned compounds may be achieved by modulating the dosing schedule such that subjects experience periodic partial or full reductions in dosing for fixed amounts of time, followed by a resumption of dosing. In some embodiments, dosages are administered daily for between one and thirty days, followed by a dosing holiday lasting for between one and thirty days. In some embodiments, during the dosing holiday, no dose is administered. In some further embodiments, the compound and its metabolites are allowed to clear completely from the subject's body prior to administration of the next dose. In some other embodiments, during the dosing holiday, a dose less than the usual daily dose is administered. In some further embodiments, an amount of the administered compound less than the therapeutically effective amount is allowed to remain within the subject during the dosing holiday. In some further embodiments, an amount of the administered compound sufficient to maintain therapeutic levels in the affected tissues is allowed to remain within the subject.

In some embodiments, the maximum serum concentration of the compound during the dosing schedule is less than 120 ng/ml, less than 100 ng/ml, less than 90 ng/ml, less than 80 ng/ml, less than 70 ng/ml, less than 60 ng/ml, or less than 50 ng/ml. In some embodiments, the minimum serum concentration during the dosing schedule is less than 10 ng/ml, less than 1 ng/ml, less than 0.1 ng/ml, less than 0.01 ng/ml, or less than 0.001 ng/ml. In some embodiments, the level of the compound administered during the dosing schedule may be undetectable during some portion of the dosing holiday.

In some embodiments, the maximum serum concentration of the compound during the dosing schedule is higher during an initial phase of administration, and lower in subsequent phases. In some embodiments, the maximum serum concentration of the compound during the initial (loading) phase of administration is less than 500 ng/ml, less than 400 ng/ml, less than 300 ng/ml, less than 200 ng/ml, less than 150 ng/ml, less than 120 ng/ml, less than 100 ng/ml, less than 90 ng/ml, less than 80 ng/ml, less than 70 ng/ml, less than 60 ng/ml, or less than 50 ng/ml. In some such embodiments, the maximum serum concentration during the initial phase of administration is from 5 ng/ml to 250 ng/ml. In some embodiments, the maximum serum concentration of the compound during the subsequent (maintenance) phase of administration is less than 350 ng/ml, less than 200 ng/ml, less than 120 ng/ml, less than 100 ng/ml, less than 90 ng/ml, less than 80 ng/ml, less than 70 ng/ml, less than 60 ng/ml, or less than 50 ng/ml, less than 40 ng/ml, less than 35 ng/ml, or less than 10 ng/ml. One of ordinary skill in the art will readily be aware of such methods as exist in the art for the monitoring of serum concentrations of pharmaceutical agents, and means of adjusting dosages of the compounds disclosed herein in order to achieve the desired serum concentrations. In some embodiments, the weekly dose to be administered is 600 mg or less. In some embodiments, the weekly dose is to be administered is 500 mg or less, 400 mg or less, 300 mg or less, 200 mg or less, 100 mg or less, 50 mg or less, 40 mg or less, 25 mg or less, 10 mg or less, or 5 mg or less, or within a range defined by any two of the foregoing.

In some embodiments, a compound of Formula I is administered wherein each R¹¹ is not hydrogen and the compound metabolizes in vivo to form a compound of Formula I wherein each R¹¹ is hydrogen or an anion of such compound. For example, in some embodiments, Compound 1 is administered and it metabolizes in vivo to form Compound 3. In some embodiments, Compound 2 is administered and it metabolizes in vivo to form Compound 4. In some such embodiments, the maximum serum concentration of compounds of Formula I wherein each R¹¹ is hydrogen or an anion of such compound (e.g., Compounds 3 or 4) during the initial (loading) phase of administration is 500 ng/ml or less, 450 ng/ml or less, 400 ng/ml or less, 350 ng/ml or less, 300 ng/ml or less, or 250 ng/ml or less. In some embodiments, the maximum serum concentration during the subsequent (maintenance) phase of administration is 500 ng/ml or less, 450 ng/ml or less, 400 ng/ml or less, 350 ng/ml or less, 300 ng/ml or less, 250 ng/ml or less, 200 ng/ml or less, 150 ng/ml or less, or 120 ng/ml or less.

According to the present disclosure, the dosing schedule may be varied in order to attain the desired therapeutic effect while eliminating HPT-related side effects. In each of the following embodiments, variations in dosing schedule as described may be repeated throughout the duration of the treatment. In each of the following embodiments, the first dosage may be higher, lower, or the same as the dosages following the first dosage. In each of the following embodiments, a loading dose may precede the disclosed dosing regimen, and a dosing holiday may or may not follow the administration of the loading dose.

In some embodiments, dosages are administered every other day for the duration of the treatment. In other embodiments, dosages are administered on two out of every three days for the duration of the treatment. In still other embodiments, dosages are administered two out of every four days for the duration of the treatment. In some embodiments, dosages are administered daily for one day, followed by a two day dosing holiday. In some embodiments, dosages are administered daily for one day, followed by a two day dosing holiday. In some embodiments, dosages are administered daily for one day, followed by a three day dosing holiday. In some embodiments, dosages are administered daily for one day, followed by a four day dosing holiday. In some embodiments, dosages are administered daily for one day, followed by a five day dosing holiday. In some embodiments, dosages are administered daily for one day, followed by a six day dosing holiday. In some embodiments, dosages are administered daily for one day, followed by a seven day dosing holiday. In some embodiments, dosages are administered daily for one day, followed by an eight day dosing holiday. In some embodiments, dosages are administered daily for one day, followed by a nine day dosing holiday. In some embodiments, dosages are administered daily for one day, followed by a ten day dosing holiday. In some embodiments, dosages are administered daily for one day, followed by an eleven day dosing holiday. In some embodiments, dosages are administered daily for one day, followed by a twelve day dosing holiday. In some embodiments, dosages are administered daily for one day, followed by a thirteen day dosing holiday. In some embodiments, dosages are administered daily for one day, followed by a fourteen day dosing holiday.

In some embodiments, dosages are administered daily for two days, followed by a one day dosing holiday. In some embodiments, dosages are administered daily for two days, followed by a two day dosing holiday. In some embodiments, dosages are administered daily for two days, followed by a three day dosing holiday. In some embodiments, dosages are administered daily for two days, followed by a four day dosing holiday. In some embodiments, dosages are administered daily for two days, followed by a five day dosing holiday. In some embodiments, dosages are administered daily for two days, followed by a six day dosing holiday. In some embodiments, dosages are administered daily for two days, followed by a seven day dosing holiday. In some embodiments, dosages are administered daily for two days, followed by an eight day dosing holiday. In some embodiments, dosages are administered daily for two days, followed by a nine day dosing holiday. In some embodiments, dosages are administered daily for two days, followed by a ten day dosing holiday. In some embodiments, dosages are administered daily for two days, followed by an eleven day dosing holiday. In some embodiments, dosages are administered daily for two days, followed by a twelve day dosing holiday. In some embodiments, dosages are administered daily for two days, followed by a thirteen day dosing holiday. In some embodiments, dosages are administered daily for two days, followed by a fourteen day dosing holiday.

In some embodiments, dosages are administered daily for three days, followed by a one day dosing holiday. In some embodiments, dosages are administered daily for three days, followed by a two day dosing holiday. In some embodiments, dosages are administered daily for three days, followed by a three day dosing holiday. In some embodiments, dosages are administered daily for three days, followed by a four day dosing holiday. In some embodiments, dosages are administered daily for three days, followed by a five day dosing holiday. In some embodiments, dosages are administered daily for three days, followed by a six day dosing holiday. In some embodiments, dosages are administered daily for three days, followed by a seven day dosing holiday. In some embodiments, dosages are administered daily for three days, followed by an eight day dosing holiday. In some embodiments, dosages are administered daily for three days, followed by a nine day dosing holiday. In some embodiments, dosages are administered daily for three days, followed by a ten day dosing holiday. In some embodiments, dosages are administered daily for three days, followed by an eleven day dosing holiday. In some embodiments, dosages are administered daily for three days, followed by a twelve day dosing holiday. In some embodiments, dosages are administered daily for three days, followed by a thirteen day dosing holiday. In some embodiments, dosages are administered daily for three days, followed by a fourteen day dosing holiday.

In some embodiments, dosages are administered daily for four days, followed by a one day dosing holiday. In some embodiments, dosages are administered daily for four days, followed by a two day dosing holiday. In some embodiments, dosages are administered daily for four days, followed by a three day dosing holiday. In some embodiments, dosages are administered daily for four days, followed by a four day dosing holiday. In some embodiments, dosages are administered daily for four days, followed by a five day dosing holiday. In some embodiments, dosages are administered daily for four days, followed by a six day dosing holiday. In some embodiments, dosages are administered daily for four days, followed by a seven day dosing holiday. In some embodiments, dosages are administered daily for four days, followed by an eight day dosing holiday. In some embodiments, dosages are administered daily for four days, followed by a nine day dosing holiday. In some embodiments, dosages are administered daily for four days, followed by a ten day dosing holiday. In some embodiments, dosages are administered daily for four days, followed by an eleven day dosing holiday. In some embodiments, dosages are administered daily for four days, followed by a twelve day dosing holiday. In some embodiments, dosages are administered daily for four days, followed by a thirteen day dosing holiday. In some embodiments, dosages are administered daily for four days, followed by a fourteen day dosing holiday.

In some embodiments, dosages are administered daily for five days, followed by a one day dosing holiday. In some embodiments, dosages are administered daily for five days, followed by a two day dosing holiday. In some embodiments, dosages are administered daily for five days, followed by a three day dosing holiday. In some embodiments, dosages are administered daily for five days, followed by a four day dosing holiday. In some embodiments, dosages are administered daily for five days, followed by a five day dosing holiday. In some embodiments, dosages are administered daily for five days, followed by a six day dosing holiday. In some embodiments, dosages are administered daily for five days, followed by a seven day dosing holiday. In some embodiments, dosages are administered daily for five days, followed by an eight day dosing holiday. In some embodiments, dosages are administered daily for five days, followed by a nine day dosing holiday. In some embodiments, dosages are administered daily for five days, followed by a ten day dosing holiday. In some embodiments, dosages are administered daily for five days, followed by an eleven day dosing holiday. In some embodiments, dosages are administered daily for five days, followed by a twelve day dosing holiday. In some embodiments, dosages are administered daily for five days, followed by a thirteen day dosing holiday. In some embodiments, dosages are administered daily for five days, followed by a fourteen day dosing holiday.

In some embodiments, dosages are administered daily for six days, followed by a one day dosing holiday. In some embodiments, dosages are administered daily for six days, followed by a two day dosing holiday. In some embodiments, dosages are administered daily for six days, followed by a three day dosing holiday. In some embodiments, dosages are administered daily for six days, followed by a four day dosing holiday. In some embodiments, dosages are administered daily for six days, followed by a five day dosing holiday. In some embodiments, dosages are administered daily for six days, followed by a six day dosing holiday. In some embodiments, dosages are administered daily for six days, followed by a seven day dosing holiday. In some embodiments, dosages are administered daily for six days, followed by an eight day dosing holiday. In some embodiments, dosages are administered daily for six days, followed by a nine day dosing holiday. In some embodiments, dosages are administered daily for six days, followed by a ten day dosing holiday. In some embodiments, dosages are administered daily for six days, followed by an eleven day dosing holiday. In some embodiments, dosages are administered daily for six days, followed by a twelve day dosing holiday. In some embodiments, dosages are administered daily for six days, followed by a thirteen day dosing holiday. In some embodiments, dosages are administered daily for six days, followed by a fourteen day dosing holiday.

In some embodiments, dosages are administered daily for seven days, followed by a one day dosing holiday. In some embodiments, dosages are administered daily for seven days, followed by a two day dosing holiday. In some embodiments, dosages are administered daily for seven days, followed by a three day dosing holiday. In some embodiments, dosages are administered daily for seven days, followed by a four day dosing holiday. In some embodiments, dosages are administered daily for seven days, followed by a five day dosing holiday. In some embodiments, dosages are administered daily for seven days, followed by a six day dosing holiday. In some embodiments, dosages are administered daily for seven days, followed by a seven day dosing holiday. In some embodiments, dosages are administered daily for seven days, followed by an eight day dosing holiday. In some embodiments, dosages are administered daily for seven days, followed by a nine day dosing holiday. In some embodiments, dosages are administered daily for seven days, followed by a ten day dosing holiday. In some embodiments, dosages are administered daily for seven days, followed by an eleven day dosing holiday. In some embodiments, dosages are administered daily for seven days, followed by a twelve day dosing holiday. In some embodiments, dosages are administered daily for seven days, followed by a thirteen day dosing holiday. In some embodiments, dosages are administered daily for seven days, followed by a fourteen day dosing holiday.

In some embodiments, dosages are administered daily for eight days, followed by a one day dosing holiday. In some embodiments, dosages are administered daily for eight days, followed by a two day dosing holiday. In some embodiments, dosages are administered daily for eight days, followed by a three day dosing holiday. In some embodiments, dosages are administered daily for eight days, followed by a four day dosing holiday. In some embodiments, dosages are administered daily for eight days, followed by a five day dosing holiday. In some embodiments, dosages are administered daily for eight days, followed by a six day dosing holiday. In some embodiments, dosages are administered daily for eight days, followed by a seven day dosing holiday. In some embodiments, dosages are administered daily for eight days, followed by an eight day dosing holiday. In some embodiments, dosages are administered daily for eight days, followed by a nine day dosing holiday. In some embodiments, dosages are administered daily for eight days, followed by a ten day dosing holiday. In some embodiments, dosages are administered daily for eight days, followed by an eleven day dosing holiday. In some embodiments, dosages are administered daily for eight days, followed by a twelve day dosing holiday. In some embodiments, dosages are administered daily for eight days, followed by a thirteen day dosing holiday. In some embodiments, dosages are administered daily for eight days, followed by a fourteen day dosing holiday.

In some embodiments, dosages are administered daily for nine days, followed by a one day dosing holiday. In some embodiments, dosages are administered daily for nine days, followed by a two day dosing holiday. In some embodiments, dosages are administered daily for nine days, followed by a three day dosing holiday. In some embodiments, dosages are administered daily for nine days, followed by a four day dosing holiday. In some embodiments, dosages are administered daily for nine days, followed by a five day dosing holiday. In some embodiments, dosages are administered daily for nine days, followed by a six day dosing holiday. In some embodiments, dosages are administered daily for nine days, followed by a seven day dosing holiday. In some embodiments, dosages are administered daily for nine days, followed by an eight day dosing holiday. In some embodiments, dosages are administered daily for nine days, followed by a nine day dosing holiday. In some embodiments, dosages are administered daily for nine days, followed by a ten day dosing holiday. In some embodiments, dosages are administered daily for nine days, followed by an eleven day dosing holiday. In some embodiments, dosages are administered daily for nine days, followed by a twelve day dosing holiday. In some embodiments, dosages are administered daily for nine days, followed by a thirteen day dosing holiday. In some embodiments, dosages are administered daily for nine days, followed by a fourteen day dosing holiday.

In some embodiments, dosages are administered daily for ten days, followed by a one day dosing holiday. In some embodiments, dosages are administered daily for ten days, followed by a two day dosing holiday. In some embodiments, dosages are administered daily for ten days, followed by a three day dosing holiday. In some embodiments, dosages are administered daily for ten days, followed by a four day dosing holiday. In some embodiments, dosages are administered daily for ten days, followed by a five day dosing holiday. In some embodiments, dosages are administered daily for ten days, followed by a six day dosing holiday. In some embodiments, dosages are administered daily for ten days, followed by a seven day dosing holiday. In some embodiments, dosages are administered daily for ten days, followed by an eight day dosing holiday. In some embodiments, dosages are administered daily for ten days, followed by a nine day dosing holiday. In some embodiments, dosages are administered daily for ten days, followed by a ten day dosing holiday. In some embodiments, dosages are administered daily for ten days, followed by an eleven day dosing holiday. In some embodiments, dosages are administered daily for ten days, followed by a twelve day dosing holiday. In some embodiments, dosages are administered daily for ten days, followed by a thirteen day dosing holiday. In some embodiments, dosages are administered daily for ten days, followed by a fourteen day dosing holiday.

In some embodiments, dosages are administered daily for eleven days, followed by a one day dosing holiday. In some embodiments, dosages are administered daily for eleven days, followed by a two day dosing holiday. In some embodiments, dosages are administered daily for eleven days, followed by a three day dosing holiday. In some embodiments, dosages are administered daily for eleven days, followed by a four day dosing holiday. In some embodiments, dosages are administered daily for eleven days, followed by a five day dosing holiday. In some embodiments, dosages are administered daily for eleven days, followed by a six day dosing holiday. In some embodiments, dosages are administered daily for eleven days, followed by a seven day dosing holiday. In some embodiments, dosages are administered daily for eleven days, followed by an eight day dosing holiday. In some embodiments, dosages are administered daily for eleven days, followed by a nine day dosing holiday. In some embodiments, dosages are administered daily for eleven days, followed by a ten day dosing holiday. In some embodiments, dosages are administered daily for eleven days, followed by an eleven day dosing holiday. In some embodiments, dosages are administered daily for eleven days, followed by a twelve day dosing holiday. In some embodiments, dosages are administered daily for eleven days, followed by a thirteen day dosing holiday. In some embodiments, dosages are administered daily for eleven days, followed by a fourteen day dosing holiday.

In some embodiments, dosages are administered daily for twelve days, followed by a one day dosing holiday. In some embodiments, dosages are administered daily for twelve days, followed by a two day dosing holiday. In some embodiments, dosages are administered daily for twelve days, followed by a three day dosing holiday. In some embodiments, dosages are administered daily for twelve days, followed by a four day dosing holiday. In some embodiments, dosages are administered daily for twelve days, followed by a five day dosing holiday. In some embodiments, dosages are administered daily for twelve days, followed by a six day dosing holiday. In some embodiments, dosages are administered daily for twelve days, followed by a seven day dosing holiday. In some embodiments, dosages are administered daily for twelve days, followed by an eight day dosing holiday. In some embodiments, dosages are administered daily for twelve days, followed by a nine day dosing holiday. In some embodiments, dosages are administered daily for twelve days, followed by a ten day dosing holiday. In some embodiments, dosages are administered daily for twelve days, followed by an eleven day dosing holiday. In some embodiments, dosages are administered daily for twelve days, followed by a twelve day dosing holiday. In some embodiments, dosages are administered daily for twelve days, followed by a thirteen day dosing holiday. In some embodiments, dosages are administered daily for twelve days, followed by a fourteen day dosing holiday.

In some embodiments, dosages are administered daily for thirteen days, followed by a one day dosing holiday. In some embodiments, dosages are administered daily for thirteen days, followed by a two day dosing holiday. In some embodiments, dosages are administered daily for thirteen days, followed by a three day dosing holiday. In some embodiments, dosages are administered daily for thirteen days, followed by a four day dosing holiday. In some embodiments, dosages are administered daily for thirteen days, followed by a five day dosing holiday. In some embodiments, dosages are administered daily for thirteen days, followed by a six day dosing holiday. In some embodiments, dosages are administered daily for thirteen days, followed by a seven day dosing holiday. In some embodiments, dosages are administered daily for thirteen days, followed by an eight day dosing holiday. In some embodiments, dosages are administered daily for thirteen days, followed by a nine day dosing holiday. In some embodiments, dosages are administered daily for thirteen days, followed by a ten day dosing holiday. In some embodiments, dosages are administered daily for thirteen days, followed by an eleven day dosing holiday. In some embodiments, dosages are administered daily for thirteen days, followed by a twelve day dosing holiday. In some embodiments, dosages are administered daily for thirteen days, followed by a thirteen day dosing holiday. In some embodiments, dosages are administered daily for thirteen days, followed by a fourteen day dosing holiday.

In some embodiments, dosages are administered daily for fourteen days, followed by a one day dosing holiday. In some embodiments, dosages are administered daily for fourteen days, followed by a two day dosing holiday. In some embodiments, dosages are administered daily for fourteen days, followed by a three day dosing holiday. In some embodiments, dosages are administered daily for fourteen days, followed by a four day dosing holiday. In some embodiments, dosages are administered daily for fourteen days, followed by a five day dosing holiday. In some embodiments, dosages are administered daily for fourteen days, followed by a six day dosing holiday. In some embodiments, dosages are administered daily for fourteen days, followed by a seven day dosing holiday. In some embodiments, dosages are administered daily for fourteen days, followed by an eight day dosing holiday. In some embodiments, dosages are administered daily for fourteen days, followed by a nine day dosing holiday. In some embodiments, dosages are administered daily for fourteen days, followed by a ten day dosing holiday. In some embodiments, dosages are administered daily for fourteen days, followed by an eleven day dosing holiday. In some embodiments, dosages are administered daily for fourteen days, followed by a twelve day dosing holiday. In some embodiments, dosages are administered daily for fourteen days, followed by a thirteen day dosing holiday. In some embodiments, dosages are administered daily for fourteen days, followed by a fourteen day dosing holiday.

In some embodiments, dosages are administered daily for thirty days followed by a thirty day dosing holiday. In some embodiments, dosages are administered daily for thirty days followed by a 25-30 day dosing holiday. In some embodiments, dosages are administered daily for thirty days followed by a 20-25 day dosing holiday. In some embodiments, dosages are administered daily for thirty days followed by a 15-20 day dosing holiday. In some embodiments, dosages are administered daily for thirty days followed by a 10-15 day dosing holiday. In some embodiments, dosages are administered daily for thirty days followed by a 5-10 day dosing holiday. In some embodiments, dosages are administered daily for thirty days followed by a 1-5 day dosing holiday.

In some embodiments, dosages are administered daily for 25-30 days followed by a thirty day dosing holiday. In some embodiments, dosages are administered daily for 25-30 days followed by a 25-30 day dosing holiday. In some embodiments, dosages are administered daily for 25-30 days followed by a 20-25 day dosing holiday. In some embodiments, dosages are administered daily for 25-30 days followed by a 15-20 day dosing holiday. In some embodiments, dosages are administered daily for 25-30 days followed by a 10-15 dosing holiday. In some embodiments, dosages are administered daily for 25-30 days followed by a 5-10 day dosing holiday. In some embodiments, dosages are administered daily for 25-30 days followed by a 1-5 day dosing holiday.

In some embodiments, dosages are administered daily for 20-25 days followed by a thirty day dosing holiday. In some embodiments, dosages are administered daily for 20-25 days followed by a 25-30 day dosing holiday. In some embodiments, dosages are administered daily for 20-25 days followed by a 20-25 day dosing holiday. In some embodiments, dosages are administered daily for 20-25 days followed by a 15-20 day dosing holiday. In some embodiments, dosages are administered daily for 20-25 days followed by a 10-15 dosing holiday. In some embodiments, dosages are administered daily for 20-25 days followed by a 5-10 day dosing holiday. In some embodiments, dosages are administered daily for 20-25 days followed by a 1-5 day dosing holiday.

In some embodiments, dosages are administered daily for 15-20 days followed by a thirty day dosing holiday. In some embodiments, dosages are administered daily for 15-20 days followed by a 25-30 day dosing holiday. In some embodiments, dosages are administered daily for 15-20 days followed by a 20-25 day dosing holiday. In some embodiments, dosages are administered daily for 15-20 days followed by a 15-20 day dosing holiday. In some embodiments, dosages are administered daily for 15-20 days followed by a 10-15 day dosing holiday. In some embodiments, dosages are administered daily for 15-20 days followed by a 5-10 day dosing holiday. In some embodiments, dosages are administered daily for 15-20 days followed by a 1-5 day dosing holiday.

In any of the forgoing embodiments, the daily dosing may be administered in one dose administered once or day, or in two or more divided doses administered multiple times per day. For example, the compounds described herein may be administered once per day, twice per day, three times per day, or four times per day.

In some embodiments, the subject's T3, T4 or TSH levels are monitored, such that administration of a daily dose can be eliminated or reduced on any day in which the T3, T4, or TSH levels are below a pre-determined threshold. When the T3, T4, or TSH levels rise above a pre-determined threshold during the dosing holiday, normal daily dosing can continue.

### EXAMPLES

### Example 1: Compound 2 Alternative Dosing Study in Dogs

The objective of the study was to determine the effects of oral administration of Compound 2 once-daily for 14 days followed by alternate day dosing for 14 days on plasma cholesterol levels and indicators of thyroid function in beagle dogs. Compound 2 was formulated with Lutrol F68 NF (Poloxomer 188) and carboxymethylcellulose (CMC; sodium salt/high viscosity) and was administered as a suspension in 0.5% CMC/1% Lutrol in deionized water. Twelve beagle dogs (9-15 kg) were randomized into 6 dosing groups (1 male and 1 female/group) and gavaged once-daily with a 0.5% CMC/1% Lutrol F68 suspension of Compound 2 at doses of 0.1, 0.3, 1, 3, or 10 mg/day or with vehicle for 14 days. At the end of the treatment cycle (Cycle 1), the dogs were washed out for 4 weeks and then entered into a second 14-day treatment cycle. Cycle 2 employed the same dosing paradigm as Cycle 1, but animals were randomized to Cycle 2 in such a way that the combined dosing groups from the two cycles consisted of 4 different animals (2 males, 2 females) each. At the conclusion of Cycle 2, dosing was continued on alternate days for an additional 14-day period (Cycle 2 Extension). Blood samples were collected at baseline and appropriate time intervals thereafter and analyzed for total plasma cholesterol levels, serum levels of total T4 (tT4), free T4 (fT4), total T3 (tT3), free T3 (fT3), and thyroid stimulating hormone (TSH).

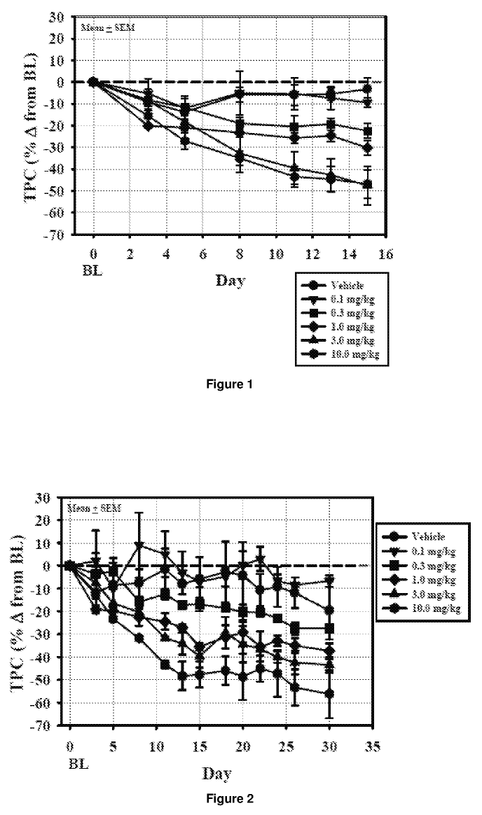

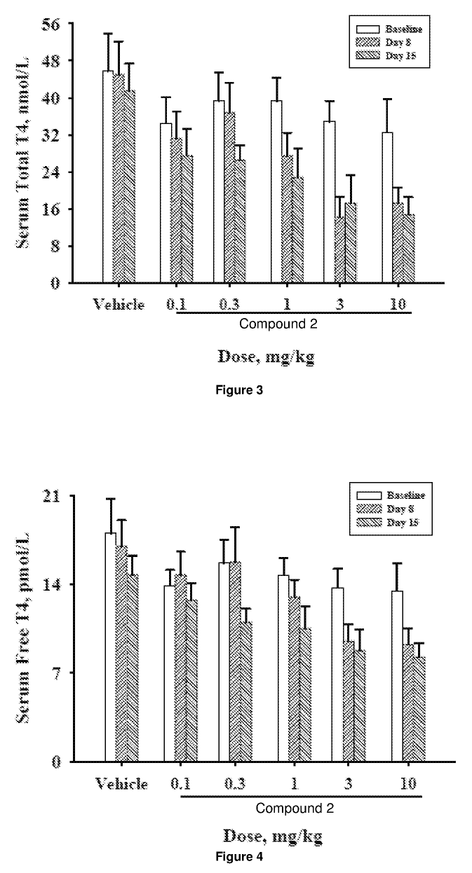

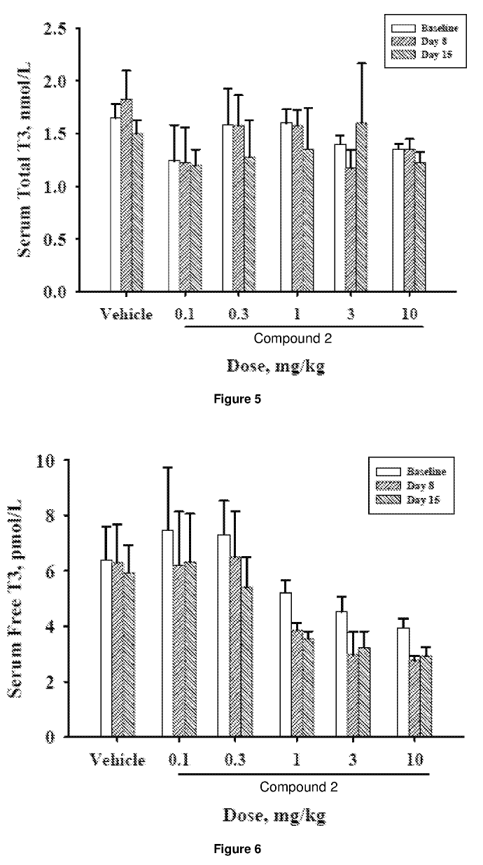

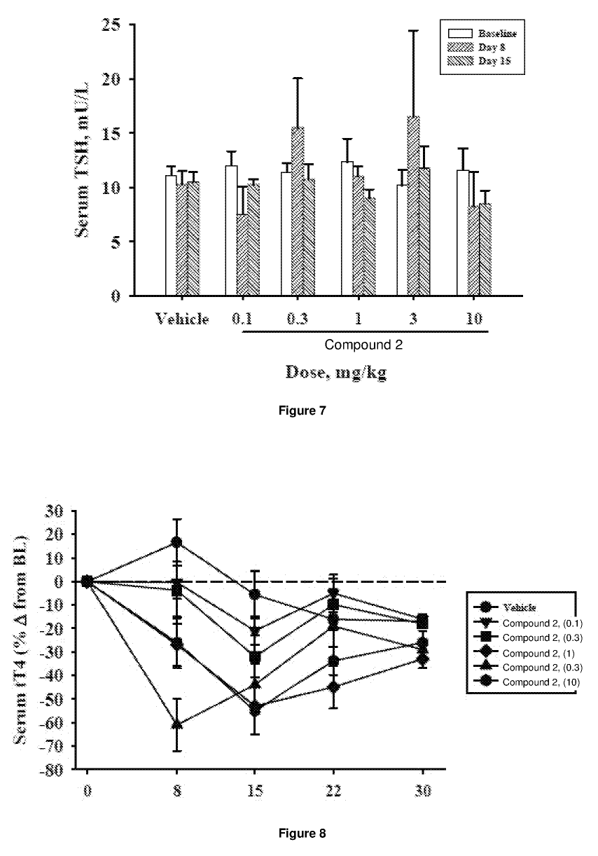

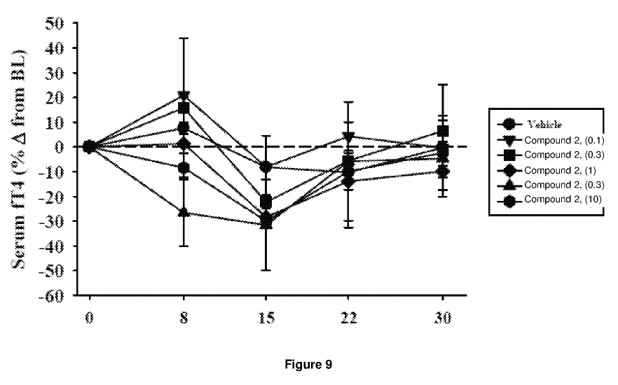

Treatment with Compound 2 for 14 days resulted in progressive, dose-dependent reductions of total plasma cholesterol levels, with an average reduction on Day 15 of −28 mg/dL or ˜22% from baseline at a dose of 0.3 mg/kg/day and of −71 mg/dL or ˜47% from baseline at the highest dose evaluated (10 mg/kg/day) (See FIG. 1). The lowest dose of Compound 2 evaluated, 0.1 mg/kg/day, had minimal effects on total plasma cholesterol levels (FIG. 1). During the alternate day dosing period of Cycle 2 (Cycle 2 Extension), total plasma cholesterol levels in the Compound 2 treatment groups remained reduced relative to vehicle-treated animals to a similar or greater extent than observed after once-daily dosing (See FIG. 2). Once-daily treatment with Compound 2 resulted in dose-dependent reductions of serum tT4 (˜20-54%, see FIG. 3), dose-related reductions of fT4 (˜8-39%, see FIG. 4), and non-dose-dependent reductions of fT3 (˜15-32%, see FIG. 6) from baseline levels. There were no meaningful changes in tT3 levels relative to baseline levels that exceeded those observed in the vehicle-treated group in any of the Compound 2 treatment groups (see FIG. 5). Effects of once-daily Compound 2 treatment on serum TSH levels were variable, with full suppression in some animals and up to 4-fold elevations in others on Day 8. On Day 15, TSH levels were reduced from baseline in a non-dose-dependent manner by ˜6-27% in the Compound 2-treated groups (see FIG. 7). During the Cycle 2 Extension, levels of tT4 and fT4 that were suppressed by once-daily treatment gradually recovered to levels that approached those of vehicle-treated animals (see FIGS. 8, 9). A more variable recovery of fT3 and TSH levels was observed that did not extend to all Compound 2 dose groups.

In conclusion, once-daily oral treatment of beagle dogs for 14 days with Compound 2 (0.1-10 mg/kg) resulted in dose-dependent reductions of average total plasma cholesterol levels (up to 47% from baseline) that were accompanied by dose-dependent reductions of serum tT4 levels, dose-related reductions in fT4 levels, and considerable fluctuations in serum TSH levels. Levels of tT3 were unaffected by treatment whereas fT3 levels were reduced in a non-dose-dependent manner. A switch from once-daily to alternate day dosing of Compound 2 in the Cycle 2 extension did not compromise cholesterol lowering efficacy but resulted in a gradual recovery of levels of tT4 and fT4 and, in some dose groups, of levels of fT3 and TSH that were suppressed by once-daily oral Compound 2 treatment. Alternate day dosing is thus an effective alternative to once-daily dosing that has reduced impact on the thyroid hormone axis.

### Example 2: Compound 1 Primary Alternative Dosing Study in Dogs

The objective of this study is to explore effects of alternative dosing regimens on various clinical parameters in Beagle Dogs. 5 beagle dogs per group of a single sex are randomly placed into five groups. One group receives daily dosing of vehicle only. Test groups receive either 1) daily dosing of test article; 2) one day dosing followed by a one day dosing holiday; 3) one day dosing followed by a two day dosing holiday; 4) three days consecutive dosing followed by a four day dosing holiday; or 5) five days consecutive dosing followed by a two day dosing holiday. Dogs are sourced from a from a non-naïve colony. Dosing is by a single daily administration. Test article is administered at a dose of 10 mg/kg of Compound 1. Treatment is by oral gavage, once daily on each treatment day, and the duration period is three weeks (21 days). No recovery period is used. The vehicle for administration is 0.5% CMC/1% Kolliphor P188 in deionized water, which is prepared once weekly and is refrigerated. Food consumption is not monitored and veterinary examination is not performed unless needed. Blood is drawn from each subject 7 days before initiation of dosing, 4 days before initiation of dosing, and 2, 4, 6, 8, 10, 12, 14, 16, 18, 20, and 22 days after dose initiation. Subjects are assessed for their plasma cholesterol levels, Aspartate transaminase (AST) and alanine transaminase (ALT) levels, and thyroid function. Total T4, Total T3, Free T4, Free T3, and Thyroid Stimulating Hormone are assessed. After 22 days, data are compiled and subjected to appropriate statistical analyses.

### Example 3: Compound 1 Secondary Alternative Dosing Study in Dogs

The objective of this study is to explore effects of alternative dosing regimens on various clinical parameters in Beagle Dogs. 4, 5, or 6 beagle dogs per group of a single sex are randomly placed into five groups. One group receives daily dosing of vehicle only. Test groups receive either 1) daily dosing of test article; 2) one day dosing followed by a two day dosing holiday; 3) three days consecutive dosing followed by a four day dosing holiday; or 4) four days consecutive dosing followed by a three day dosing holiday. Dogs are sourced from a from a non-naïve colony. Treatment is by oral gavage, once daily on each treatment day, and the duration period is three weeks. No recovery period is used. The vehicle for administration is 0.5% CMC/1% Lutrol F68 in deionized water, which is prepared once weekly and is refrigerated. Food consumption is not monitored and veterinary examination is not performed unless needed. Blood is drawn from each subject every two days or every 3-4 days. Subjects are assessed for their plasma cholesterol levels, Aspartate transaminase (AST) and alanine transaminase (ALT) levels, and thyroid function. Total T4, Total T3, Free T4, Free T3, and Thyroid Stimulating Hormone are assessed. After 14 days, and again after 22 days, data are compiled and subjected to appropriate statistical analyses.

### Example 4: Compound 1 Alternative Dosing Study in Humans

A phase 2, randomized, double-blind, placebo-controlled, multicenter study is carried out to assess the efficacy, safety, and tolerability of Compound 1, administered for 12 weeks followed by a 4-week off-drug phase, in patients with primary hypercholesterolemia and non-alcoholic fatty liver disease. Objectives of the study are 1) to evaluate the effects of Compound 1 compared to placebo on LDL-C after 12 weeks of treatment; 2) to evaluate the effects of Compound 1 compared to placebo on liver fat content, measured by magnetic resonance imaging—Proton Density Fat Fraction (MRI-PDFF); 3) to evaluate the percent change from baseline in hepatic stiffness at Week 12 as assessed by MR Elastography (MRE); 4) to evaluate changes in other lipid parameters including total cholesterol (TC), triglycerides (TG), non-high-density lipoprotein cholesterol (non-HDL-C), HDL-C, very low-density lipoprotein cholesterol (VLDL-C), apolipoprotein B (apo B), and lipoprotein (a), [Lp(a)]; and 5) to evaluate duration of effect by safety and lipid assessments 4-weeks post-treatment. Subjects are selected who have at least 10% liver fat as assessed by MRI-Proton Density Fat Fraction (PDFF), are 18-75 years old at the time of screening, have a body mass index (BMI) 18.50-40.00 kg/m2, have a fasting serum LDL-C>130 mg/dL at screening or >110 on lipid lowering medications, have free T3 and free T4 within the normal range, and have baseline values of AST, ALT, ALP and total bilirubin established by at least 2 samples at least several weeks (i.e. 4-12 weeks) apart where differences in the levels of those repeat measures is <20%. Subjects included in the study must also meet any three of the following criteria 1) type 2 diabetes receiving prescription medication or HbAlc>5.7; 2) triglycerides >150 mg/dL or who are receiving prescription medication for elevated triglycerides; 3) systolic blood pressure >130 mmHg or diastolic blood pressure >85 mmHg or who are receiving prescription medication for hypertension; 4) waist circumference >40 inches (men) or >35 inches (women); or 5) HDL<40 mg/dL (men) or <50 md/dL (women), or who are receiving prescription medication for low HDL. Subjects are randomly assigned to one of four individual treatment groups: daily placebo orally (PO); daily Compound 1 dose 5 mg PO; daily Compound 1 dose 10 mg PO; and every other day (QOD) Compound 1 Dose 10 mg PO. Compound 1 is provided in the form of a tablet containing either 5 mg or 10 mg of Compound 1.

Subjects are assessed for their total thyroid function at various points during the treatment course, at the end of treatment, and four weeks after treatment. In examining total thyroid function, total T4, Total T3, Free T4, Free T3, and Thyroid Stimulating Hormone are assessed. Subjects are also monitored periodically for indicators of cardiac side effects, such as by monitoring of blood pressure, C-reactive protein, cardiac troponin (cTnI), creatine kinase (CK), Creatine Kinase MB Isoenzyme, by periodic electrocardiogram and/or use of a Holter monitor, or by other means as appropriate. Evaluation of thyroid function, cardiac health, and clinical endpoints are made periodically during the treatment course. Final measurements are made at the cessation of dosing at 12 weeks, and after the four week washout period at 16 weeks. After 16 weeks, data are compiled and subjected to appropriate statistical analyses.

### Example 5: Compound 1 Second Alternative Dosing Study in Humans

A phase 2, randomized, double-blind, placebo-controlled, multicenter study is carried out to assess the efficacy, safety, and tolerability of Compound 1, administered for 12 weeks followed by a 4-week off-drug phase, in patients with primary hypercholesterolemia and non-alcoholic fatty liver disease. Objectives of the study are 1) to evaluate the effects of Compound 1 compared to placebo on LDL-C after 12 weeks of treatment; 2) to evaluate the effects of Compound 1 compared to placebo on liver fat content, measured by magnetic resonance imaging—Proton Density Fat Fraction (MRI-PDFF); 3) to evaluate the percent change from baseline in hepatic stiffness at Week 12 as assessed by MR Elastography (MRE); 4) to evaluate changes in other lipid parameters including total cholesterol (TC), triglycerides (TG), non-high-density lipoprotein cholesterol (non-HDL-C), HDL-C, very low-density lipoprotein cholesterol (VLDL-C), apolipoprotein B (apo B), and lipoprotein (a), [Lp(a)]; and 5) to evaluate duration of effect by safety and lipid assessments 4-weeks post-treatment. Subjects are selected who have at least 10% liver fat as assessed by MRI-Proton Density Fat Fraction (PDFF), are 18-75 years old at the time of screening, have a body mass index (BMI) 18.50-40.00 kg/m2, have a fasting serum LDL-C>130 mg/dL at screening or >110 on lipid lowering medications, have free T3 and free T4 within the normal range, and have baseline values of AST, ALT, ALP and total bilirubin established by at least 2 samples at least several weeks (i.e. 4-12 weeks) apart where differences in the levels of those repeat measures is <20%. Subjects included in the study must also meet any three of the following criteria 1) type 2 diabetes receiving prescription medication or HbAlc>5.7; 2) triglycerides >150 mg/dL or who are receiving prescription medication for elevated triglycerides; 3) systolic blood pressure >130 mmHg or diastolic blood pressure >85 mmHg or who are receiving prescription medication for hypertension; 4) waist circumference >40 inches (men) or >35 inches (women); or 5) HDL<40 mg/dL (men) or <50 md/dL (women), or who are receiving prescription medication for low HDL. Subjects are randomly assigned to one of seven individual treatment groups: daily placebo orally (PO); 250 mg Loading dose of Compound 1 followed by daily Compound 1 dose 5 mg PO; 250 mg Loading dose of Compound 1 followed by daily Compound 1 dose 10 mg PO; 250 mg Loading dose of Compound 1 followed by every other day (QOD) Compound 1 Dose 10 mg PO; 100 mg Loading dose of Compound 1 followed by daily Compound 1 dose 5 mg PO; 100 mg Loading dose of Compound 1 followed by daily Compound 1 dose 10 mg PO; and 100 mg Loading dose of Compound 1 followed by every other day (QOD) Compound 1 Dose 10 mg PO. Compound 1 is provided in the form of a tablet containing either 5 mg or 10 mg of Compound 1.

Subjects are assessed for their total thyroid function at various points during the treatment course, at the end of treatment, and four weeks after treatment. In examining total thyroid function, total T4, Total T3, Free T4, Free T3, and Thyroid Stimulating Hormone are assessed. Subjects are also monitored periodically for indicators of cardiac side effects, such as by monitoring of blood pressure, C-reactive protein, cardiac troponin (cTnI), creatine kinase (CK), Creatine Kinase MB Isoenzyme, by periodic electrocardiogram and/or use of a Holter monitor, or by other means as appropriate. Evaluation of thyroid function, cardiac health, and clinical endpoints are made periodically during the treatment course. Final measurements are made at the cessation of dosing at 12 weeks, and after the four week washout period at 16 weeks. After 16 weeks, data are compiled and subjected to appropriate statistical analyses.

### Example 6: Compound 1 Alternative Dosing Study in Humans

A phase 1b, randomized, double-blind, placebo-controlled, study is carried out to assess the efficacy, safety, and tolerability of Compound 1, administered for 12 weeks followed by a 4-week off-drug phase, in patients with primary hypercholesterolemia and non-alcoholic fatty liver disease.

The maximum cumulative weekly dose will not exceed 40 mg in this current clinical study. The doses of 5 mg daily and up to 40 mg once weekly of Compound 1 over the 4-week treatment period in this Phase 1b study produce dose and/or schedule dependent decreases in LDL-C and triglycerides. The projected systemic exposure (AUC0-168 hr [i.e., exposure during one week]) of the active metabolite at the 40 mg once weekly dose is 5570 ng*hr/mL. Pharmacokinetic modeling shows the AUC0-168 hr of 18300 ng*hr/mL. Projected exposures in humans receiving 40 mg once weekly is therefore estimated to be about 3-fold lower than exposures at the previously determined 5 mg/kg/day no observable adverse effect level (NOAEL).

Approximately 32 subjects are enrolled and randomly assigned in a 6:2 ratio (Compound 1:placebo) to one of the following treatment groups: (1) 5 mg Compound 1 or Placebo once daily (2) 20 mg Compound 1 or Placebo once weekly (3) 40 mg Compound 1 or Placebo once weekly (4) 10 mg Compound 1 or Placebo every other day The primary endpoint is the percent change in LDL-C from the baseline visit to Day 29. Either a 20% decrease in LDL-C or a lowering of LDL-C to within the normal range is considered clinically meaningful. Subjects are also monitored for adverse effects and/or clinically relevant changes in health status or results from laboratory analyses. Each subject will be screened for changes in health status or laboratory results 28 days after the last administration of the test article.

Clinical parameters to be observed during the trial may include one or more of: Physical Exam; monitoring of concomitant medications; assessment/monitoring of hyperthyroidism/thyrotoxicosis; vitals (sitting >5 minutes); weight; assessment of alcohol intake; assessment/monitoring of lifestyle habits; 12-lead ecg (supine >10 minutes); assessment/monitoring of drug compliance; AE/SAE assessment; and/or 24-hr Holter monitoring. A complete physical examination includes, but is not limited to: general appearance, skin, HEENT (includes neck/throat exam to assess thyroid), respiratory examination, cardiovascular assessment including rhythm and presence of any cardiac, abnormalities (eg, gallops, murmurs, cardiomegaly), abdominal examination, musculoskeletal, neurological examination to record the presence of abnormalities in mental, status, motor, and sensory function (including reflex exam), gastrointestinal, genitourinary if appropriate, and/or any additional assessments necessary to establish baseline status or evaluate, symptoms or adverse experiences. Additionally, laboratory tests may be conducted including any one or more of the following: hematology, including platelet count, WBC with differential (% and absolute counts), hematocrit, hemoglobin, RBCs, Mean Corpuscular Volume (MCV), mean corpuscular hemoglobin concentration (MCHC), and mean cell hemoglobin (MCH); lipid profile (blinded after randomization), including LDL-C direct via beta-quantification, non-HDL-C, VLDL-C(calculated), total cholesterol, triglycerides, HDL-C, Lp(a), and apo B; serum chemistry, including BUN, creatinine, calcium, glucose, sodium, potassium, chloride, bicarbonate, phosphorus, uric acid, and creatinine kinase; liver panel, including AST (SGOT), ALT (SGPT), alkaline phosphatase (ALP), and bilirubin (total, direct, indirect); additional liver tests, including INR/PT, total protein, albumin, sex hormone binding globulin (SHBG), and thyroxine-binding globulin (TBG), thyroid panel (blinded after randomization), including TSH, total and free T4, and total and free T3; cardiac biomarkers, including CK, CK-MB, and cardiac troponin I (cTnI); urinalysis, including color, appearance specific gravity pH, protein, leucocyte esterase, glucose, ketones blood, bilirubin urobilinogen, nitrates, and if abnormal, microscopic analysis; other samples, including FSH (postmenopausal females), serum pregnancy, serology for HbsAg, HCV, and HIV, HbAlC, fasting insulin and glucose for HOMA, alcohol and drug testing, and PK samples. Additional clinical parameters may be monitored or assessed as necessary.

What is claimed is:

1. A method of treating a disease or condition, comprising the steps, in order, of:
administering for a first number of days to a subject in need thereof a first daily amount of a compound;
ceasing administration of the compound or administering a second daily amount of the compound for a second number of days, wherein the second daily amount of the compound is less than the first daily amount; and
administering a third daily amount of the compound for a third number of days to the subject,
wherein the disease or condition is selected from the group consisting of obesity, hyperlipidemia, hypercholesterolemia, diabetes, non-alcoholic fatty liver disease, non-alcoholic steatohepatitis, atherosclerosis, cardiovascular disease, hypothyroidism, and thyroid cancer; and
wherein said compound has the structure of Formula I: 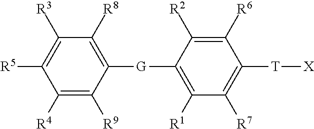
wherein:
G is selected from the group consisting of —O—, —S—, —S(═O)—, —S(═O)₂—, —Se—, —CH₂—, —CF₂—, —CHF—, —C(O)—, —CH(OH)—, —CH(C₁-C₄ alkyl)-, —CH(C₁-C₄ alkoxy)-, —C(═CH₂)—, —NH—, and —N(C₁-C₄ alkyl)-;
T is selected from the group consisting of —(CR^{a}₂)ₖ—, —CR^{b}═CR^{b}—(CR^{a}₂)ₙ—, —(CR^{a}₂)ₙ—CR^{b}═CR^{b}—, —(CR^{a}₂)—CR^{b}═CR^{b}—(CR^{a}₂)—, —O(CR^{b}₂)(CR^{a}₂)ₙ—, —S(CR^{b}₂)(CR^{a}₂)ₙ—, N(R^{C})(CR^{b}₂)(CR^{a}₂)ₙ—, N(R^{b})C(O)(CR^{a}₂)ₙ, —C(O)(CR^{a}₂)ₘ—, —(CR^{a}₂)ₘC(O)—, —(CR^{a}₂)C(O)(CR^{a}₂)ₙ, —(CR^{a}₂)ₙC(O)(CR^{a}₂)—, and —C(O)NH(CR^{b}₂)(CR^{a}₂)ₚ;
k is an integer from 1-4;
m is an integer from 0-3;
n is an integer from 0-2;
p is an integer from 0-1;
each R^{a} is independently selected from the group consisting of hydrogen, optionally substituted —C₁-C₄ alkyl, halogen, —OH, optionally substituted —O—C₁-C₄ alkyl, —OCF₃, optionally substituted —S—C₁-C₄ alkyl, —NR^{b}R^{c}, optionally substituted —C₂-C₄ alkenyl, and optionally substituted —C₂-C₄ alkynyl; with the proviso that when one R^{a} is attached to C through an O, S, or N atom, then the other R^{a} attached to the same C is a hydrogen, or attached via a carbon atom;
each R^{b} is independently selected from the group consisting of hydrogen and optionally substituted —C₁-C₄ alkyl;
each R^{c} is independently selected from the group consisting of hydrogen and optionally substituted —C₁-C₄ alkyl, optionally substituted —C(O)—C₁-C₄ alkyl, and —C(O)H;
R¹, and R² are each independently selected from the group consisting of halogen, optionally substituted —C₁-C₄ alkyl, optionally substituted —S—C₁-C₃ alkyl, optionally substituted —C₂-C₄ alkenyl, optionally substituted —C₂-C₄ alkynyl, —CF₃, —OCF₃, optionally substituted-O—C₁-C₃ alkyl, and cyano;
R⁶, R⁷, R⁸, and R⁹ are each independently selected from the group consisting of hydrogen, halogen, optionally substituted —C C₁-C₄ alkyl, optionally substituted —S—C₁-C₃ alkyl, optionally substituted —C₂-C₄ alkenyl, optionally substituted —C₂-C₄ alkynyl, —CF₃, —OCF₃, optionally substituted-O—C₁-C₃ alkyl, and cyano; or R⁶ and T are taken together along with the carbons they are attached to form a ring of 5 to 6 atoms including 0 to 2 heteroatoms independently selected from —NRⁱ—, —O—, and —S—, with the proviso that when there are 2 heteroatoms in the ring and both heteroatoms are different than nitrogen then both heteroatoms have to be separated by at least one carbon atom; and X is attached to this ring by a direct bond to a ring carbon, or by —(CR^{a}₂)— or —C(O)— bonded to a ring carbon or a ring nitrogen;
Rⁱ is selected from the group consisting of hydrogen, —C(O)C₁-C₄ alkyl, —C₁-C₄ alkyl, and —C₁-C₄-aryl;
R³ and R⁴ are independently selected from the group consisting of hydrogen, halogen, —CF₃, —OCF₃, cyano, optionally substituted —C₁-C₁₂ alkyl, optionally substituted —C₂-C₁₂ alkenyl, optionally substituted —C₂-C₁₂ alkynyl, —SR^{d}, —S(═O)R^{e}, —S(═O)₂R^{e}, —S(═O)₂NR^{f}R^{g}, —C(O)OR^{h}, —C(O)R^{e}, —N(R^{b})C(O)NR^{f}R^{g}, —N(R^{b})S(═O)₂R^{e}, —N(R^{b})S(═O)₂NR^{f}R^{g}, and —NR^{f}R^{g};
each R^{d} is selected from the group consisting of optionally substituted —C₁-C₁₂ alkyl, optionally substituted —C₂-C₁₂ alkenyl, optionally substituted —C₂-C₁₂ alkynyl, optionally substituted —(CR^{b}₂)ₙ aryl, optionally substituted —(CR^{b}₂)ₙ cycloalkyl, optionally substituted —(CR^{b}₂)ₙ heterocycloalkyl, and —C(O)NR^{f}R^{g};
each R^{e} is selected from the group consisting of optionally substituted —C₁-C₁₂ alkyl, optionally substituted —C₂-C₁₂ alkenyl, optionally substituted —C₂-C₁₂ alkynyl, optionally substituted —(CR^{a}₂)ₙ aryl, optionally substituted —(CR^{a}₂)ₙ cycloalkyl, and optionally substituted —(CR^{a}₂)ₙ heterocycloalkyl;
R^{f} and R^{g} are each independently selected from the group consisting of hydrogen, optionally substituted —C₁-C₁₂ alkyl, optionally substituted —C₂-C₁₂ alkenyl, optionally substituted —C₂-C₁₂ alkynyl, optionally substituted —(CR^{b}₂)ₙ aryl, optionally substituted —(CR^{b}₂)ₙ cycloalkyl, and optionally substituted —(CR^{b}₂)ₙ heterocycloalkyl, or R^{f} and R^{g} may together form an optionally substituted heterocyclic ring, which may contain a second heterogroup selected from the group consisting of O, NR^{c}, and S, wherein said optionally substituted heterocyclic ring may be substituted with 0-4 substituents selected from the group consisting of optionally substituted —C₁-C₄ alkyl, —OR^{b}, oxo, cyano, —CF₃, optionally substituted phenyl, and —C(O)OR^{h};
each R^{h} is selected from the group consisting of optionally substituted —C₁-C₁₂ alkyl, optionally substituted —C₂-C₁₂ alkenyl, optionally substituted —C₂-C₁₂ alkynyl, optionally substituted —(CR^{b}₂)ₙ aryl, optionally substituted —(CR^{b}₂)ₙ cycloalkyl, and optionally substituted —(CR^{b}₂)ₙ heterocycloalkyl;
R⁵ is selected from the group consisting of —OH, optionally substituted —OC₁-C₆ alkyl, OC(O)R^{e}, —OC(O)OR^{h}, —F, —NHC(O)R^{e}, —NHS(═O)R^{e}, —NHS(═O)₂R^{e}, —NHC(═S)NH(R^{h}), and —NHC(O)NH(R^{h});
X is P(O)YR¹¹Y′R¹¹;
Y and Y′ are each independently selected from the group consisting of —O—, and —NR^{v}—; when Y and Y′ are —O—, R¹¹ attached to —O— is independently selected from the group consisting of —H, alkyl, optionally substituted aryl, optionally substituted heterocycloalkyl, optionally substituted CH₂-heterocycloakyl wherein the cyclic moiety contains a carbonate or thiocarbonate, optionally substituted -alkylaryl, —C(R^{z})₂OC(O)NR^{z}₂, —NR^{z}—C(O)—R^{y}, —C(R^{z})₂—OC(O)R^{y}, —C(R^{z})₂—O—C(O)OR^{y}, —C(R^{z})₂OC(O)SR^{y}, -alkyl-S—C(O)R^{y}, -alkyl-S—S-alkylhydroxy, and -alkyl-S—S—S-alkylhydroxy;
when Y and Y′ are —NR^{v}—, then R¹¹ attached to —NR^{v}— is independently selected from the group consisting of —H, —[C(R^{z})₂]_{q}—COOR^{y}, —C(R^{x})₂COOR^{y}, —[C(R^{z})₂]_{q} C(O)SR^{y}, and -cycloalkylene-COOR^{y};
when Y is —O— and Y′ is NR^{v}, then R¹¹ attached to —O— is independently selected from the group consisting of —H, alkyl, optionally substituted aryl, optionally substituted heterocycloalkyl, optionally substituted CH₂-heterocycloakyl wherein the cyclic moiety contains a carbonate or thiocarbonate, optionally substituted -alkylaryl, —C(R^{z})₂OC(O)NR^{z}₂, —NR^{z}—C(O)—R^{y}, —C(R^{z})₂—OC(O)R^{y}, —C(R^{z})₂—O—C(O)OR^{y}, —C(R^{z})₂OC(O)SR^{y}, -alkyl-S—C(O)R^{y}, -alkyl-S—S-alkylhydroxy, and -alkyl-S—S—S-alkylhydroxy; and R¹¹ attached to —NR^{v}— is independently selected from the group consisting of H, —[C(R^{z})₂]_{q} COOR^{y}, —C(R^{x})₂COOR^{y}, —[C(R^{z})₂]_{q}—C(O)SR^{y}, and -cycloalkylene-COOR^{y};
or when Y and Y′ are independently selected from —O— and NR^{v}, then together R¹¹ and R¹¹ are -alkyl-S—S-alkyl- to form a cyclic group, or together R¹¹ and R¹¹ are the group: 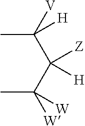
wherein:
V, W, and W′ are independently selected from the group consisting of hydrogen, optionally substituted alkyl, optionally substituted aralkyl, heterocycloalkyl, aryl, substituted aryl, heteroaryl, substituted heteroaryl, optionally substituted 1-alkenyl, and optionally substituted 1-alkynyl;
or together V and Z are connected via an additional 3-5 atoms to form a cyclic group containing 5-7 atoms, wherein 0-1 atoms are heteroatoms and the remaining atoms are carbon, substituted with hydroxy, acyloxy, alkylthiocarbonyloxy, alkoxycarbonyloxy, or aryloxycarbonyloxy attached to a carbon atom that is three atoms from both Y groups attached to the phosphorus;
or together V and Z are connected via an additional 3-5 atoms to form a cyclic group, wherein 0-1 atoms are heteroatoms and the remaining atoms are carbon, that is fused to an aryl group at the beta and gamma position to the Y attached to the phosphorus;
or together V and W are connected via an additional 3 carbon atoms to form an optionally substituted cyclic group containing 6 carbon atoms and substituted with one substituent selected from the group consisting of hydroxy, acyloxy, alkoxycarbonyloxy, alkylthiocarbonyloxy, and aryloxycarbonyloxy, attached to one of said carbon atoms that is three atoms from a Y attached to the phosphorus;
or together Z and W are connected via an additional 3-5 atoms to form a cyclic group, wherein 0-1 atoms are heteroatoms and the remaining atoms are carbon, and V must be aryl, substituted aryl, heteroaryl, or substituted heteroaryl;
or together W and W′ are connected via an additional 2-5 atoms to form a cyclic group, wherein 0-2 atoms are heteroatoms and the remaining atoms are carbon, and V must be aryl, substituted aryl, heteroaryl, or substituted heteroaryl;
Z is selected from the group consisting of —CHR^{z}OH, —CHR^{z}OC(O)R^{y}, —CHR^{z}OC(S)R^{y}, —CHR^{z}OC(S)OR^{y}, —CHR^{z}OC(O)SR^{y}, —CHR^{z}OCO₂R^{y}, —OR^{z}, —SR^{z}, —CHR^{z}N₃, —CH₂-aryl, —CH(aryl)OH, —CH(CH═CR^{z}₂)OH, —CH(C≡CR^{z})OH, —R^{z}, —NR^{z}₂, —OCOR^{y}, —OCO₂R^{y}, —SCOR^{y}, —SCO₂R^{y}, —NHCOR^{z}, —NHCO₂R^{y}, —CH₂NH-aryl, —(CH₂)q-OR^{z}, and —(CH₂)q-SR^{z};
q is an integer 2 or 3;
each R^{z} is selected from the group consisting of R^{y} and —H;
each R^{y} is selected from the group consisting of alkyl, aryl, heterocycloalkyl, and aralkyl;
each R^{x} is independently selected from the group consisting of —H, and alkyl, or together R^{x} and R^{x} form a cyclic alkyl group; and
each R^{v} is selected from the group consisting of —H, lower alkyl, acyloxyalkyl, alkoxycarbonyloxyalkyl, and lower acyl;
and pharmaceutically acceptable salts and prodrugs thereof; and pharmaceutically acceptable salts of said prodrugs.

2. The method of claim 1 wherein the compound to be administered comprises one or more of the compounds having a structures selected from the group consisting of: 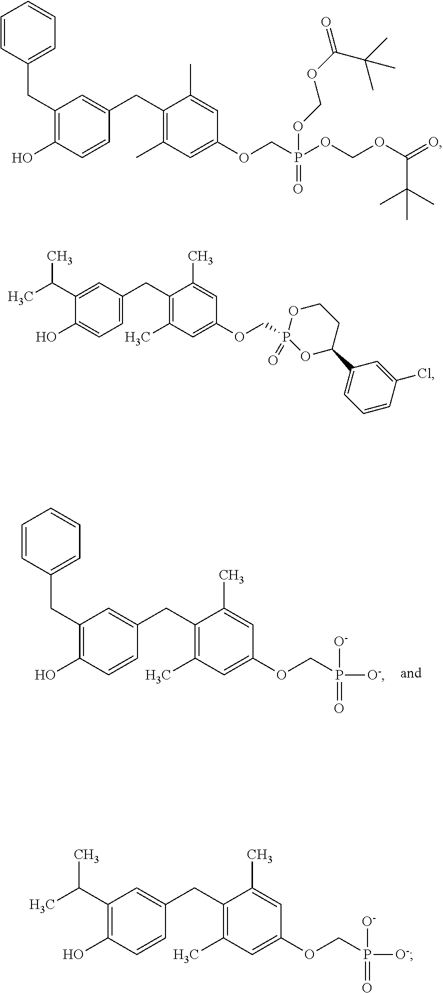
or pharmaceutically acceptable salts thereof.

3. The method of claim 1, wherein the first and third daily amounts are the same.

4. The method of claim 1, wherein the third daily amount is less than the first daily amount.

5. The method of claim 1, wherein the second and third daily amounts are the same.

6. The method of any one of claims 1-4, wherein the third daily amount is greater than the second daily amount.

7. The method of any one of claims 1-6, wherein the first and third number of days are the same.

8. The method of any one of claims 1-7, wherein the first, second, and third number of days are the same.

9. The method of any one of claims 1-6, wherein the third number of days is less than the first number of days.

10. The method of any one of claims 1-9, wherein the first, second, and third number of days are independently selected from 1 to 90.

11. The method of any one of claims 1-9, wherein the first, second, and third number of days are independently selected from 1 to 30.

12. The method of any one of claims 1-9, wherein the first, second, and third number of days are independently selected from 1 to 20.

13. The method of any one of claims 1-9, wherein the first, second, and third number of days are independently selected from 1 to 10.

14. The method of any one of claims 1-9, wherein the first, second, and third number of days are independently selected from 1 to 5.

15. The method of any one of claims 1-9, wherein the first and third number of days is 1 and the second number of days is 1.

16. The method of any one of claims 1-6, wherein the first and third number of days is 1 and the second number of days is 2.

17. The method of any one of claims 1-6, wherein the first and third number of days is 3 and the second number of days is 4.

18. The method of any one of claims 1-6, wherein the first and third number of days is 4 and the second number of days is 3.

19. The method of any one of claims 1-9, wherein the first and third number of days is 4 and the second number of days is 4.

20. The method of any one of claims 1-6, wherein the first and third number of days is 5 and the second number of days is 4.

21. The method of any one of claims 1-6, wherein the first and third number of days is 4 and the second number of days is 5.

22. The method of any one of claims 1-9, wherein the first and third number of days is 10 and the second number of days is 10.

23. The method of any one of claims 1-9, wherein the first and third number of days is 30 and the second number of days is 30.

24. The method of any one of claims 1-6, wherein the first and third number of days is 2 and the second number of days is 1.

25. The method of any one of claims 1 to 24, wherein the administration during the first and third number of days is once per day.

26. The method of any one of claims 1 to 25, comprising ceasing administration of the compound for the second number of days.

27. The method of any one of claims 1 to 25, comprising administering the second daily amount of the compound for the second number of days.

28. The method of any one of claims 1 to 27, comprising monitoring the subject's T3, T4 or TSH levels and ceasing administration of the compound or administering the second daily amount of the compound when said T3, T4, or TSH levels are below a first threshold value and resuming administration of the compound at the first daily amount when said T3, T4, or TSH levels are above a second threshold value.

29. The method of claim 28, wherein the first and second threshold values are the same.

30. The method of any of claims 1-29 wherein the total weekly dosage of the compound during the first number of days is from 40 to 150 mg.

31. The method of any of claims 1-29 wherein the total weekly dosage of the compound during the first number of days is from 50 to 90 mg.

32. The method of any of claims 1-29 wherein the total weekly dosage of the compound during the first number of days is from 60 to 80 mg.

33. The method of any of claims 1-29 wherein the weekly dosage of the compound during the first number of days is from 5 to 250 mg.

34. The method of any of claims 1-33 wherein the maximum serum concentration of the compound during the third number of days is 100 ng/mL or less.

35. The method of any of claims 1-33 wherein the maximum serum concentration of the compound during the entire treatment period is 100 ng/mL or less.

36. The method of any of claims 1-35 wherein, the compound administered has the structure: 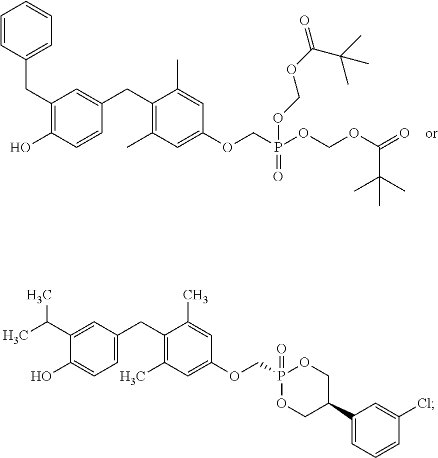
and the maximum serum concentration of a compound having the structure: 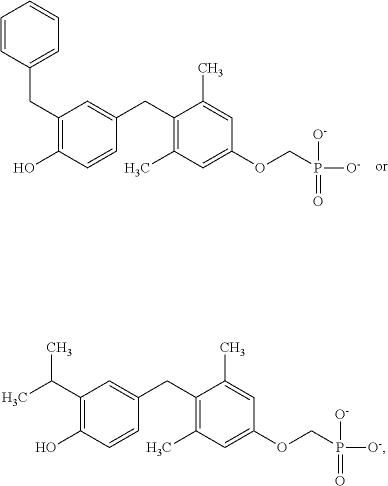
during the entire treatment period is 500 ng/mL or less.

37. The method of claim 1, wherein the compound administered has the structure of Formula I wherein each R¹¹ is not hydrogen, and the maximum serum concentration of a compound having the structure of Formula I wherein each R¹¹ is hydrogen, or an anion thereof, during the entire treatment period is 500 ng/mL or less.

38. The method of claim 1 wherein the first and third number of days is 30 and the second number of days is 30.

39. The method of claim 1, comprising:
ceasing administration of the compound or administering the second daily amount of the compound for a fourth number of days;
administering the third daily amount of the compound for a fifth number of days; and
repeating said ceasing administration or administering the second daily amount for the fourth number of days, and said administering the third daily amount of the compound for the fifth number of days.
